(19) United States
(12) Patent Application Publication
(10) Pub. No.: US 2017/0160171 A1
(43) Pub. Date: Jun. 8, 2017

(54) MULTIPLEX IMMUNOHISTOCHEMISTRY IMAGE CYTOMETRY

(71) Applicant: OREGON HEALTH AND SCIENCE UNIVERSITY, Portland, OR (US)

(72) Inventors: Takahiro Tsujikawa, Portland, OR (US); Lisa M. Coussens, Portland, OR (US); Rohan Borkar, Hillsboro, OR (US); Vahid Azimi, Hillsboro, OR (US); Sushil Kumar, Portland, OR (US); Ganapati Srinivasa, Hillsboro, OR (US)

(21) Appl. No.: 15/355,933

(22) Filed: Nov. 18, 2016

Related U.S. Application Data
(60) Provisional application No. 62/257,926, filed on Nov. 20, 2015.
(60) Provisional application No. 62/368,818, filed on Jul. 29, 2016.

(51) Int. Cl. G01N 1/30; G01N 33/68; G01N 35/00; G06T 7/33; G06T 7/11; G06K 9/46; G06T 7/90; G06T 7/00; G06T 5/50; G01N 1/44; G06T 7/136

(52) U.S. Cl. CPC G01N 1/30; G01N 1/44; G01N 33/6845; G01N 35/00029; G06T 7/337; G06T 7/136; G06K 9/4652; G06T 7/90; G06T 7/0014; G06T 5/50; G06T 7/11; G01N 2001/302; G01N 2035/00138; G01N 2035/00356; G06T 2207/20152; G06T 2207/10024; G06T 2207/30024; G06T 2207/30096; G06T 2207/20132; G06T 2207/20224

(57) ABSTRACT

Immunohistochemical (IHC) techniques that enable the sequential evaluation of at least seven biomarkers in one formalin-fixed paraffin-embedded (FFPE) tissue section are disclosed. The methods involve high-throughput multiplexed, quantitative IHC imaging, sequential IHC with iterative labeling, digital scanning, image coregistration and merging, and subsequent stripping of sections.

### RELATED APPLICATIONS

This application claims priority to U.S. Provisional Patent Application 62/257,926, filed on 20 Nov. 2015 and U.S. Provisional Patent Application 62/368,818 filed on 29 Jul. 2016, both entitled MULTIPLEX IMMUNOHISTOCHEMISTRY IMAGE CYTOMETRY and both incorporated by reference herein in their entirety.

### ACKNOWLEDGEMENT OF GOVERNMENT SUPPORT

Work resulting in the disclosed invention was supported by the United States government under the terms of grants UL1TR000128 and P30 CA 069533-17 awarded by the National Institutes of Health. The United States government has certain rights in the invention.

### FIELD

Generally, the field is immunohistochemistry. More specifically, the field is multiplex immunohistochemistry.

### BACKGROUND

Histopathological evaluation of biomarkers in formalin-fixed paraffin-embedded (FFPE) tissue sections is widely used as a diagnostic tool, as well as for prospective assessments for risk stratification based on predictive biomarkers (Ludwig and Weinstein, Nat Rev Cancer 5, 845-856 (2005); incorporated by reference herein). For solid tumors, current biomarkers encompass neoplastic cell-intrinsic factors as well as extrinsic factors emanating from the tumor microenvironment (TME) such as diverse assemblages of immune cells (Palucka and Coussens, Cell 164, 1233-1247 (2016); incorporated by reference herein). While some retrospective studies have identified new TME-based targets for therapy, others have revealed the diagnostic and prognostic power of evaluating TME and immune complexity to predict outcome and to identify tumor characteristics that, when stratified based on molecular analyses, exhibit a significantly improved response to therapy (DeNardo et al, Cancer Disc 1, 54-67 (2011); Galon et al, Science 313, 1960-1964 (2006); Fridman et al, Nat Rev Cancer 12, 298-306 (2012); and Ruffell et al, Cancer Cell 26, 623-637 (2014); all of which are incorporated by reference herein).

For scenarios such as these, a small piece of tissue is dehydrated, embedded in formalin, blocked and microtome sectioned to enable immunohistochemistry (IHC)-based assessments. FFPE tissue sections are typically evaluated one biomarker at a time, or where possible, multiplexed to enable simultaneous evaluation of 2-3 biomarkers using traditional chromogen-based IHC methods, or up to 7 simultaneous biomarkers if using non-overlapping spectral immunofluorescence (IF) (Stack et al, Methods 70, 46-58 (2014); incorporated by reference herein). Conventional IHC and multiplexed IF analyses such as these are limited in scope in part based on the species in which antibodies (primary and secondary) are produced, or by spectral overlap of fluorescence ‘tags’, respectively. These technical limitations minimize the ability to simultaneously profile multiple biomarkers in a single tissue section. They also necessitate use of adjacent tissue sections which in turn results in a difficulty in aligning and co-registering subsequent images. Further, the profiling of multiple markers on a tissue section results in the use of fewer FFPE sections—thereby preserving a precious resource.

Although 60-plus multiplexed imaging approaches based on dye-conjugated antibody or imaging mass cytometry have emerged (Gerdes et al, Proc Natl Acad Sci USA 110, 11982-11987 (2013) and Angleo et al, Nat Med 20, 436-442 (2014); both of which are incorporated by reference herein) their high cost and imaging instrumental complexity limit general use for routine analyses. While polychromatic flow cytometry (FACS) gets around many of these issues where sufficient tissue is available, and enables evaluation of up to 18-fluorescent epitopes, or more if using single cell mass cytometry, e.g., cytometry by time-of-flight (CyTOF) (Bendall et al, Science 332, 687-696 (2011); incorporated by reference herein), these approaches also have limitations in that single cell suspensions are required, thus tissue architecture is lost.

### SUMMARY

Given the practical limitations of conventional IHC and IF approaches, disclosed herein is optimized sequential IHC detection with iterative labeling, digital scanning and subsequent stripping of tissue sections, to enable simultaneous evaluation of at least 12 biomarkers in a single formalin fixed paraffin embedded (FFPE) tissue section. Particular embodiments include evaluation of up to 60 biomarkers in a FFPE tissue section.

Also disclosed herein are quantitative analytic tools, based on single-cell segmentation in digitized sequential IHC images to enable multiparameter cytometric image analysis of 16 different cell lineages.

Disclosed are methods of performing serial immunohistochemistry (IHC) on a single FFPE tissue section. The methods involve contacting the section with a tissue antigen specific antibody, contacting the section with a labeled antibody (conjugated to an enzyme label) and contacting the section with a colorimetric substrate of the enzyme label. The methods further involve generating a digital image of the section. These acts complete a staining cycle. The methods further involve heating the section to at least 90° C. for a sufficient time to remove the first tissue specific antibody from cells in the section that express the tissue specific antigen. The methods further involve performing a second staining cycle that involves contacting the section with another (preferably a different) tissue specific antigen, a labeled antibody with an enzyme label, and a colorimetric substrate of the enzyme label and generating a digital image of the section. The heating of the section is performed between the first and the second staining cycles.

The methods can further involve heating the section to at least 90° C. after the second staining cycle and performing a third, fourth, fifth, sixth, seventh, eighth, ninth, tenth, eleventh, twelfth, or additional staining cycles provided that heating the section to at least 90° C. is performed between the third and fourth, fourth and fifth, fifth and sixth, sixth and seventh, seventh and eighth, eighth and ninth, ninth and tenth, tenth and eleventh, eleventh and twelfth, or after and between additional staining cycles. Particular embodiments include 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, or more staining cycles.

The methods can further involve coregistering the first, second, third, fourth, fifth, sixth, seventh, eighth, ninth, tenth, eleventh, twelfth, or more digital images into a composite image.

The methods can further involve staining the section with a stain that allows visualization of cellular structures such as cytoplasm, nuclei, or cell membranes, thereby generating a structure-stained image. For example, hematoxylin can be used to generate a structure-stained image. The structure-stained image can be used to perform cell segmentation and/or tissue segmentation.

The methods can further involve, after heating the section to at least 90° C., maintaining the section at a temperature of at least 90° C. for at least 15 minutes. The heating can be performed using a microwave oven or placing the section into a heat bath. The methods can further involve heating the section in a citrate buffer, including a citrate buffer in a pH range of 5.5-6.5.

The methods can involve any of a number of enzyme labels including horseradish peroxidase, alkaline phosphatase, glucose oxidase, and β-galactosidase. The colorimetric substrates can include ABTS, OPD, TMB, 4CN, DAB, AEC, BCIP, NBT, (ora BCIP/NBT mixture), and/or X-gal alone or in combination.

The methods contemplate a tissue antigen specific antibody directly conjugated to a labeled antibody such that the tissue antigen specific antibody and the labeled antibody are the same antibody.

The methods can involve the tissue section being provided as a tissue microarray.

The methods can involve one or more of: contacting the section with the first tissue antigen specific antibody, contacting the section with the first labeled antibody, contacting the section with the colorimetric substrate, destaining the colorimetric substrate, or heating the section by an automated methodology using, for example, a robot arm, a liquid handling system, or an automated fill mechanism.

It is an object of the invention to provide multiparameter cytometric analysis without requiring additional instrumentation.

It is an object of the invention to provide a multiparameter cytometric analysis that enables large-scale studies without significant cost.

It is an object of the invention to stratify patients and thus guide therapy to improve clinical outcomes.

### BRIEF DESCRIPTION OF THE SEVERAL VIEWS OF THE DRAWINGS

Many of the drawings submitted herein are better understood in color, which is not available in patent application publications at the time of filing. Applicants consider the color versions of the drawings as part of the original submission and reserve the right to present color images of the drawings in later proceedings.

FIGS. 1A-1F. FIG. 1A is a set of digital scans representing bright field sequential IHC of one single formalin-fixed paraffin embedded (FFPE) section of human head and neck squamous cell carcinoma (HNSCC) tissue revealing staining characteristics of the indicated antigens. Primary antibodies were visualized with horseradish peroxidase-conjugated polymer and 3-amino-9-ethylcarbazole (AEC) detection followed by whole slide digital scanning. Following destaining in an alcohol gradient and a heat-based antibody stripping protocol using citrate pH 6.0, samples were restained sequentially with the indicated panels for lymphoid biomarkers. FIG. 1B is a set of digital scans representing bright field sequential IHC of one single formalin-fixed paraffin embedded (FFPE) section of human head and neck squamous cell carcinoma (HNSCC) tissue revealing staining characteristics of the indicated antigens. Primary antibodies were visualized with horseradish peroxidase-conjugated polymer and 3-amino-9-ethylcarbazole (AEC) detection followed by whole slide digital scanning. Following destaining in an alcohol gradient and a heat-based antibody stripping protocol using citrate pH 6.0, samples were restained sequentially with the indicated panels for myeloid biomarkers. FIG. 1C is a diagram that illustrates image coregistration—following manual selection of single cell or structure indicated by the circles, the XY coordinates of scanned images were calculated and utilized for adjustment of alignment by using CellProfiler. FIG. 1D is a diagram that further illustrates image coregistration—AEC color signals were extracted from each digitized single marker image by using the ImageJ plugin, Color Deconvolution, followed by inversion and pseudo-coloring in ImageJ. The boxes indicate the magnified area indicated by the circle in FIG. 1C. FIG. 1E is a merged composite image of an FFPE section of a head and neck squamous cell carcinoma stained with the lymphoid panel of FIG. 1A above. FIG. 1F is a merged composite image of an FFPE section of a head and neck squamous cell carcinoma (a section serial to that shown in FIG. 1E) stained with the myeloid panel of FIG. 1B above.

FIGS. 2A and 2B. FIG. 2A is an FFPE section of human HNSCC tissues analyzed by the two 12-marker panels of lineage-selective antibodies to identify lymphoid cells. Immune cell phenotypes including CD8, TH0, TH1, TH2, TH17, TREG, B cell, NK, CD163+ and CD163− TAMs, CD83+ and DC-SIGN+ DC, CD83+ granulocytes, and mast cells can be discerned (right panels with colored arrows) and classified based on expression characteristics of immune cell phenotypes, as shown in FIG. 23. Biomarkers and colors are shown in the center. Scale bars=25 μm. FIG. 2B is an FFPE section of human HNSCC tissues analyzed by the two 12-marker panels of lineage-selective antibodies to identify myeloid cells. Immune cell phenotypes including CD8, TH0, TH1, TH2, TH17, TREG, B cell, NK, CD163+ and CD163− TAMs, CD83+ and DC-SIGN+ DC, CD83+ granulocytes, and mast cells can be discerned (right panels with colored arrows) and classified based on expression characteristics of immune cell phenotypes, as shown in FIG. 23. Biomarkers and colors are shown in the center. Scale bars=25 μm.

FIGS. 3A-3D. FIG. 3A is an illustration of the process by which a hematoxylin-stained image used for automated cell segmentation based on the watershed segmentation algorithms by CellProfiler is generated. Segmentation results were utilized as templates for quantification of serially scanned AEC images, and pixel intensities of chromogenic signals and area-shape measurements were extracted and recorded by single cell-basis together with location in original images. FIG. 3B is a set of plots and images illustrating the use of single cell-based chromogenic signal intensity, cell size/area, and location to produce density plots similar to flow cytometry by using a flow and image cytometry data analysis software, FCS Express 5 Image Cytometry Version 5.01.0029 (De Novo Software). Three dot plots shown at top represent image cytometric analysis in a p16+ HNSCC tissue. Gated cell populations of CD45+ CD3+ CD8+ T cells, CD45+ CD3+ CD8− Foxp3+, and CD45+ CD3+ CD8− Foxp3−, CD45− p16+ cells are shown (middle) as an image plot with coloring of orange, magenta, green, and cyan, respectively. A 5-color multiplex IHC image corresponding to the image plot is shown at bottom, revealing matched identification between image cytometry and visualized images. The boxes depict magnified areas. Scale bars=100 μm (low magnification) and 10 μm (high magnification). FIG. 3C is a set of plots showing Image cytometry-based cell population analyses for the lymphoid biomarker panel. The markers used for identification of cell lineages are shown in FIG. 23. Gating thresholds for qualitative identification were determined based on data in negative controls (FIGS. 10B, 10C). FIG. 3D is a set of plots showing Image cytometry-based cell population analyses for the myeloid biomarker panel. The markers used for identification of cell lineages are shown in FIG. 23. Gating thresholds for qualitative identification were determined based on data in negative controls (FIGS. 10B, 10C).

FIGS. 4A-4G. FIG. 4A is an image of an FFPE sections from a HNSCC-assembled TMA including HPV-negative (N=17), HPV-positive oropharyngeal tumor (N=21) and normal oropharynx (N=8) were stained using the lymphoid biomarker panel. Scale bar=1 mm. FIG. 4B is an image of an FFPE sections from a HNSCC-assembled TMA including HPV-negative (N=17), HPV-positive oropharyngeal tumor (N=21) and normal oropharynx (N=8) were stained using the myeloid biomarker panel. Scale bar=1 mm. FIG. 4C is a heat map of cell densities (cells/mm2) of 15 immune cell lineages in each single core quantified using image cytometry. Data sets from the two panels reflecting lymphoid and myeloid biomarkers were normalized based on CD45+ cell number. A heat map according to color scale (upper left) is shown with a dendrogram of unsupervised hierarchical clustering, depicting lymphoid-, non-, and myeloid-inflamed subgroups (groups A, B, and C at bottom). FIG. 4D is a set of two box and whiskers plots showing immune cell densities of lymphoid and myeloid cell lineages comparing subgroups identified in FIG. 4C. Bars, boxes and whiskers represent median, interquartile range and range, respectively. FIG. 4E is a plot showing ratios of cell percentages comparing subgroups are shown. The bars show the median with interquartile range. FIG. 4F is a survival plot of a Kaplan-Meier analysis of postoperative survival of HNSCC patients stratified by subgroups. Statistical significance was determined via log-rank test. FIG. 4G is a plot of immune cell percentages quantified as a percentage of total CD45+ cells. For FIGS. 4D, 4E, and 4G, statistical differences were determined via Kruskal-Wallis tests with false discovery rate (FDR) adjustments, with *P<0.05, **P<0.01, ***P<0.001, and ****P<0.0001.

FIGS. 5A-5F. FIG. 5A is a set of eight images and four bar graphs showing neoplastic cell marker IHC images (p16 for HPV-positive, and EpCAM for HPV/p16-negative HNSCC) (top panels) that were utilized for semi-automated tissue segmentation classifying into neoplastic cell nests (N), intratumoral stroma (S), and blank regions (middle panels). Percentages of CD45+, CD45− neoplastic cell marker−, and neoplastic cell marker+ cells were analyzed by image cytometry, validating categorization of neoplastic cells into tumor nest regions (bottom panels).

FIG. 5B is a plot of leukocyte composition in intratumoral stroma and neoplastic cell nest regions. *P<0.05, **P<0.01, and ****P<0.0001 by Wilcoxon signed rank tests with FDR adjustments. FIG. 5C is a series of images from an HPV-positive HNSCC tissue section showing differential distribution of Tbet+ CD3+ (TH1) and GATA3+ CD3+ (TH2) cells. Left and middle panels show multi-plex IHC images with biomarker color annotations. Right panels depict cell identification and location. Boxes represent magnified areas below. Scale bars=50 μm. FIG. 5D is a plot of the ratios of TH1 to TH2, comparing intratumoral stroma and neoplastic cell nests. ****P<0.0001 by Wilcoxon signed rank test. FIG. 5E is a plot showing Kaplan-Meier analysis of postoperative survival of HNSCC patients stratified by CD66b+ granulocytes (Gr) % of total CD45+ cells infiltrated in neoplastic cell nest regions (cut-off=median). Statistical significance was determined via log-rank test. FIG. 5F is a plot of a Spearman correlation coefficient and estimated regression line, showing an inverse correlation between TH1/TH2 ratio and CD66b+ Gr % of CD45+ in neoplastic cell nest regions.

FIGS. 6A-6H. FIG. 6A is an image of PD-L1 expression on neoplastic cells in HPV-positive HNSCC tissue. The box denotes the area magnified in the right panel. Scale bars=50 μm. FIG. 6B is a set of micrographs showing PD-L1+ immune cells (red arrowheads) in 20 μm square frames. FIG. 6C is a box and whiskers plot showing PD-L1-positive % in each cell lineage was quantified by image cytometry. Bars, boxes and whiskers represent median, interquartile range and range, respectively. *P<0.05, and **P<0.01, by Kruskal-Wallis tests with FDR adjustments. FIG. 6D is an 11-color composite (left) and selective four markers (right) with colored arrowheads of CD3+ T cells (white), PD-1+(green), and PD-L1+ cells (red), showing distribution of PD-L1+ cells and PD-1+ CD3+ T cells in HNSCC tissue. Scale bars=50 μm. FIG. 6E is set of two plots showing Spearman correlations of cell densities of CD8 versus PD-L1+ CD83+ dendritic cells (DC) and PD-L1+ CD163+ tumor-associated macrophages (TAM) in HNSCC tissues (N=38) were shown with estimated regression lines (red). FIG. 6F is a set of four images—the left panels show PD-L1+ cells and the right panels show CD8+ T cells within 10 or 20 μm distance to PD-L1+ cells. Boxes represent area magnified below. Scale bars=20 μm. FIG. 6G is a plot of leukocyte composition within 20 and 10 μm-distance to PD-L1+ cells were compared with whole tissue-based composition. FIG. 6H is a set of two plots showing CD8 densities and TH1/TH2 ratios, reflecting distance to PD-L1+ cells. Statistical significance in FIGS. 6G and 6H was determined via Wilcoxon signed rank tests with FDR adjustments, with *P<0.05, and **P<0.01.

FIGS. 7A and 7B. FIG. 7A is a single channel image of the lymphoid biomarker panel. Scale bars=500 μm (left) and 100 μm (right), and magnified frames are 20 μm square. FIG. 7B is a single channel image of the myeloid biomarker panel. Scale bars=500 μm (left) and 100 μm (right), and magnified frames are 20 μm square.

FIGS. 8A-8C, FIG. 8A is a set of images of sequential IHC—following AEC wash and antibody stripping, complete removal of antibody and signal was confirmed by incubating with only the detection reagent and AEC in the next sequential round. FIG. 8B is a set of images comparing standard IHC and sequential IHC for detection of CD45 in human tonsil tissue is shown. Scale bars=100 μm. FIG. 8C is a plot of CD45 positive cells corresponding to those of FIG. 8B quantified using Aperio Imagescope (N=5). No significant reduction of CD45 detection was observed when comparing IHC round 1 to 13. The bars show mean±SD. Statistical significance determined by Kruskal-Wallis test.

FIGS. 9A and 9B. FIG. 9A is a single channel image in support of FIG. 2A. Scale bars=50 μm. FIG. 9B is a single channel image in support of FIG. 2B. Scale bars=50 μm.

FIGS. 10A-10E. FIG. 10A is a set of images and plots showing that image cytometry analysis enables visualization of positional linkage between dot plots and original images. Colored dots in the left panels are pointed out in the original AEC images (middle panels), and multiplexed IHC images (right panel). Scale bars=100 μm. Right panel shows image cytometry findings on negative control slides. FIG. 10B is a set of density plots in negative control slides in support of FIG. 3C. The x and y axes are shown on a logarithmic scale. FIG. 10C is a set of density plots in negative control slides in support of FIG. 3D. The x and y axes are shown on a logarithmic scale. FIG. 10D is a set of six plots showing the comparison between image cytometry and flow cytometry in human pancreatic ductal adenocarcinoma tissues (N=8). Representative density plots from flow cytometry (upper) and image cytometry (lower) are shown. FIG. 10E is a plot showing pairwise associations of T cell (CD45+ CD3+), B cell (CD45+ CD19+ or CD20+), CD8+ T cell (CD45+ CD3+ CD8+) as a percentage of total CD45+ cells are assessed by Spearman correlation coefficient. Estimated regression lines for each category were shown.

FIGS. 11A-110 depict a set of micrographs showing multiplex IHC findings in lymphoid-inflamed (11A), hypo-inflamed (11B), and myeloid-inflamed (11C) subgroups in HNSCC (See FIG. 4C), showing high infiltration of lymphoid cell populations in core #32 (11A), hypo-infiltration of CD45+ leukocytes in core #3 (11B), and high infiltration of CD68+ and CD66b+ cells in core #21 (11C). Boxes and hashed lines represent area magnified. Biomarkers and color annotations were shown in bottom left. Scale bars=500 μm (top left) and 100 μm (top right and bottom right). Image cytometry-based quantification was shown in corresponding to IHC images. Top two panels show density plots of CD45 and cocktail antibodies of CD3, CD20 and CD56 (lymphoid cell markers). Image plots (bottom left) depict location of cells identified above by image cytometry, according to color markers below. Composition graphs (bottom right) show quantified cell percentages of CD45−, CD45+CD3−CD20−CD56− (non-lymphoid) and CD45+ CD3−CD20− CD56+ (lymphoid) cells of total cells, according to color markers below.

FIGS. 12A-12D. FIG. 12A is a set of box-whisker plots of cell density in support of FIG. 4C. *, **, and *** show P<0.05, 0.01, and 0.001, respectively, by Kruskal-Wallis tests with FDR adjustments. Bars, boxes and whiskers represent median, interquartile range and range, respectively. FIG. 12B is a box and whisker plots comparing gene expression between HPV-positive and HPV-negative HNSCC from The Cancer Genome Atlas (N=39 and 80, respectively). TCGA HNSCC mRNA gene expression by pancan-normalized RNAseq (Illumina-HiSeq) (N=564) were downloaded from UCSC cancer browser (data obtained in October, 2014). 434 samples without available information of HPV-status were excluded, and total of 119 cases were analyzed by expression of immune cell lineage markers. Vertical axis shows log 2-based gene expression normalized to all TCGA cancer types. Bars, boxes and whiskers represent median, interquartile range and range, respectively. *, **, ***, and **** show P<0.05, 0.01, 0.001, and 0.0001, respectively, by Kruskal-Wallis tests with FDR adjustments. FIG. 12C is a plot of the area of neoplastic cell nest (% of total tissue area) compared among the three subgroups indicated in FIG. 4C. FIG. 12D is a plot of the area of neoplastic cell next (% of total tissue area) stratified by HPV status. For FIGS. 12C and 12D, each single dot represents one core/individual in the TMA. Statistical significance was determined by a Kruskal-Wallis test, and the p-values in FIG. 12C were adjusted by FDR.

FIGS. 13A-13F. FIG. 13A is a set of six single channel images in support of FIG. 5B. Boxes represent magnified areas below, and scale bars=50 μm. FIG. 13B is a plot of leukocyte composition as shown in FIG. 5C limited to HPV-positive samples. FIG. 13C is a plot of leukocyte composition as shown in FIG. 5C limited to HPV-negative samples. FIG. 13D is a set of two plots showing ratios of cell percentages of TH1 to TH2 and CD163− TAM to CD163+ TAM of intratumoral stroma (S) stratified by HPV status. FIG. 13E is a set of two plots showing ratios of cell percentages of TH1 to TH2 and CD163− TAM to CD163+ TAM of neoplastic cell nest regions (N) stratified by HPV-status. For both FIGS. 13D and 13E *P<0.05, and **P<0.01, by Wilcoxon signed rank test. FIG. 13F is a plot of a Kaplan-Meier analysis of postoperative survival of HNSCC patients stratified by CD66b+ granulocyte % of total CD45+ cells infiltrated in whole tissue, intratumoral stroma (S), and neoplastic cell nest regions (N) (N=37). The median value of percentages was chosen as a cutoff point. Statistical significance was determined via log-rank test.

FIGS. 14A-14E. FIG. 14A is a set of images of PD-1 expressing lineages in human HNSCC tissues. Green arrowheads indicate PD-1+ cells, and lineage markers identifying CD8, TH1, TH2, TREG, TH17, TH0, and B cell are shown. Top and bottom panels are shown in 20 μm square frames. FIG. 14B is a box and whiskers plot of the percentage of PD-1-positive cells in each cell lineage quantified by image cytometry, comparing HPV-positive, HPV-negative HNSCC, and normal pharynx. Bars, boxes and whiskers represent median, interquartile range and range, respectively. Statistical significance was determined via Kruskal-Wallis tests with FDR adjustments, with *P<0.05, and **P<0.01. FIG. 14C is a set of multiplex IHC images from the same field corresponding to the images in FIG. 6D. Red and white arrowheads in the left panel show PD-L1 expression on CD45+ and CD45− cells, respectively. White and red arrowheads in the middle panel represent CD3+ CD8− and CD3+ CD8+ cells, respectively, while green frames indicate PD-1 expression. The right panel represents a composite of PD-1 CD8, Tbet, GATA3, Foxp3 colocalized in CD3+ cells. Scale bars=50 μm. FIG. 14D is a graph of the percentage of PD-L1 positive cells in each cell lineage quantified by image cytometry, in comparison between intratumoral stroma and neoplastic cell nest regions. Bars, boxes and whiskers represent median, interquartile range and range, respectively. Statistical significance was determined via Wilcoxon signed rank tests with FDR adjustments, with *P<0.05. FIG. 14E is a graph of the percentage of PD-1 positive cells in each cell lineage quantified by image cytometry, in comparison between intratumoral stroma and neoplastic cell nest regions. Bars, boxes and whiskers represent median, interquartile range and range, respectively. Statistical significance was determined via Wilcoxon signed rank tests with FDR adjustments, with *P<0.05.

FIGS. 15A and 15B. FIG. 15A is a correlation of cell densities between 16 cell lineages and PDL1+ CD83+ DC were analyzed by Spearman correlation coefficients (N=38) *, **, and *** are P<0.05, 0.01 and 0.001 respectively. FIG. 15B is a correlation of cell densities between 16 cell lineages and PDL1+ CD163+ TAM were analyzed by Spearman correlation coefficients (N=38) *, **, and *** are P<0.05, 0.01 and 0.001 respectively.

FIG. 16 is a flowchart of an exemplary method to co-register a set of images of AEC-stained samples to a reference image (e.g., a structure-stained image such as a hematoxylin image) in accordance with the disclosure.

FIG. 17 is a flowchart of an exemplary method to perform cell segmentation and quantification in accordance with the disclosure.

FIG. 18 is a flowchart of an exemplary method to visualize extracted AEC data as a composite pseudo-color image in accordance with the disclosure.

FIG. 19 is a flowchart of an exemplary method to perform tissue segmentation and quantification in accordance with the disclosure.

FIG. 20 is a flowchart of an exemplary workflow to perform the quantitative and visualization procedures in accordance with the disclosure.

FIG. 21 is a flowchart of an exemplary workflow in accordance with the disclosure showing representative output and input files generated during the as part of the quantitative and visualization procedures described herein.

FIGS. 22A and 22B are tables showing sequential IHC protocol and antibody information for a lymphoid panel (FIG. 22A) and a myeloid panel (FIG. 22B).

FIG. 23 is a table showing biomarkers used to identify cell lineages.

FIG. 24 is a table showing patient and disease characteristics.

FIG. 25 is a table showing variables Associated with Overall Survival without Adjustment: Cox Regression Analysis.

FIG. 26 is a table showing variables Associated with Overall Survival with adjustment for HPV-status: Cox Regression Analysis.

FIG. 27 describes a multiplexed IHC protocol allowing staining of, for example, 60 biomarkers (e.g., tissue specific antigens) in a FFPE tissue section.

### DETAILED DESCRIPTION

### I. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), 5 The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below.

As will be understood by one of ordinary skill in the art, each embodiment disclosed herein can comprise or consist of its particular stated element, step, ingredient or component. Thus, the terms “include” or “including” should be interpreted to recite: “comprise, or consist of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified.

In addition, the materials, methods, and examples are illustrative only and not intended to be limiting. In order to facilitate review of the various embodiments of the disclosure, the following explanations of specific terms are provided:

Antibody: A polypeptide including at least a light chain or heavy chain immunoglobulin variable region which specifically recognizes and binds an epitope of an antigen or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody. The VH and VL regions can be further segmented into complementarity determining regions (CDRs) and framework regions. The CDRs (also termed hypervariable regions) are the regions within the VH and VL responsible for antibody binding.

The term “antibody” encompasses intact immunoglobulins, as well the variants and portions thereof, such as Fab fragments, Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker. In dsFvs the chains have been mutated to introduce a disulfide bond to stabilize the association of the chains. The term also includes genetically engineered forms such as chimeric antibodies, heteroconjugate antibodies (such as, bispecific antibodies). Diabodies include two epitope-binding sites that may be bivalent. See, for example, EP 0404097; WO1993/01161; and Holliger, et al., Proc. Natl. Acad. Sci. USA 90 (1993) 6444-6448. Antibody fragments can also include isolated CDRs. For a review of antibody fragments, see Hudson, et al., Nat. Med. 9 (2003) 129-134. See also, Pierce Catalog and Handbook, 1994-1995 (Pierce Chemical Co., Rockford, Ill.); Kuby, J., Immunology, 3rd Ed., W.H. Freeman & Co., New York, 1997. The term also includes monoclonal antibodies (all antibody molecules have the same VH and VL sequences and therefore the same binding specificity) and polyclonal antisera (the antibodies vary in VH and VL sequence but all bind a particular antigen such as a tissue antigen.) Antibodies to carry out the methods disclosed herein are available from a number of commercial sources (e.g., Abcam, Sigma-Aldrich, etc.). Similarly, the tissue antigens described herein are known in the art. Sequence and structural information for each is readily available in publicly-available databases.

Array: An arrangement of molecules, such as biological macromolecules (such as peptides or nucleic acid molecules) or biological samples (such as tissue sections), in addressable locations on or in a substrate.

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays may be computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Tissue arrays, also called tissue microarrays or TMAs, include a plurality of sections of normal and/or diseased tissue (such as cancerous tissue with or without associated normal adjacent tissue) on a single microscope slide. A tissue microarray allows for the analysis of expression of one or more markers on a large number of tumors in a single experiment.

Contacting: Placement in conditions under which direct physical association occurs, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject. Contacting can include contacting a liquid (that liquid including one or more antibodies) with a tissue section such as a tissue section on a glass slide.

Immunohistochemistry (IHC): A technique used to identify a specific molecule in different types of tissue, including cancer tissue. Tissues in a tissue section (such as a paraffin, fixed, unfixed, frozen section, including a FFPE section) on a microscope slide are treated with an antibody that binds to the specific molecule. The antibodies are conjugated to a label that renders tissues that bound to the label visible under a microscope. Examples of labels that may be used in IHC include fluorescent dyes, radioisotopes, metals (such as colloidal gold,) and enzymes that produce a local color change upon interaction with a substrate. Multiple molecules may be assessed in the same tissue using differentially labeled antibodies—for example, by using a first antibody specific for a first molecule conjugated to a label that fluoresces at a particular wavelength and a second antibody specific for a second molecule conjugated to a label that fluoresces at a different wavelength than the one conjugated to the first molecule.

Label: A label may be any substance capable of aiding a machine, detector, sensor, device, column, or enhanced or unenhanced human eye in differentiating a labeled composition from an unlabeled composition. Labels may be used for any of a number of purposes and one skilled in the art will understand how to match the proper label with the proper purpose. Examples of uses of labels include purification of biomolecules, identification of biomolecules, detection of the presence of biomolecules, detection of protein folding, and localization of biomolecules within a cell, tissue, or organism. Examples of labels include radioactive isotopes or chelates thereof; dyes (fluorescent or nonfluorescent), stains, enzymes, nonradioactive metals, magnets, protein tags, any antibody epitope, any specific example of any of these; any combination between any of these, or any label now known or yet to be disclosed. A label may be covalently attached to a biomolecule or bound through hydrogen bonding, Van Der Waals or other forces. A label may be covalently or otherwise bound to the N-terminus, the C-terminus or any amino acid of a polypeptide or the 5′ end, the 3′ end or any nucleic acid residue in the case of a polynucleotide.

One particular example of a label is a small molecule fluorescent dye. Such a label can be conjugated to an antibody such as an antibody that binds an antigen such as a tissue antigen. One of skill in the art would be able to identify and select any appropriate fluorescent dye or combination of fluorescent dyes for use in the disclosed methods.

Another particular example of a label is an enzyme. In specific examples, the enzyme is conjugated to an antibody that specifically binds an antigen such as a tissue antigen. In still other examples, the enzyme is conjugated to a secondary antibody that specifically binds the antibody that binds the tissue antigen. After an enzyme labeled antibody is bound, a specific substrate for the enzyme is then added to the antibody. In some examples, the activity of the enzyme in the presence of the specific substrate results in a color change that indicates the presence of the label. Such a reaction can be termed a chromogenic reaction. Examples of enzyme labels include horseradish peroxidase, alkaline phosphatase, glucose oxidase, and β-galactosidase.

Another particular example of a label is a protein tag. A protein tag includes a sequence of one or more amino acids that may be used as a label as discussed above, particularly for use in protein purification. In some examples, the protein tag is covalently bound to the polypeptide. It may be covalently bound to the N-terminal amino acid of a polypeptide, the C-terminal amino acid of a polypeptide or any other amino acid of the polypeptide. Often, the protein tag is encoded by a polynucleotide sequence that is immediately 5′ of a nucleic acid sequence coding for the polypeptide such that the protein tag is in the same reading frame as the nucleic acid sequence encoding the polypeptide. Protein tags may be used for all of the same purposes as labels listed above and are well known in the art. Examples of protein tags include chitin binding protein (CBP), maltose binding protein (MBP), glutathione-S-transferase (GST), poly-histidine (His), thioredoxin (TRX), FLAG®, V5, c-Myc, HA-tag, and so forth.

A His-tag facilitates purification and binding to on metal matrices, including nickel matrices, including nickel matrices bound to solid substrates such as agarose plates or beads, glass plates or beads, or polystyrene or other plastic plates or beads. Other protein tags include BCCP, calmodulin, Nus, Thioredoxin, Streptavidin, SBP, and Ty, or any other combination of one or more amino acids that can work as a label described above.

Another particular example of a label is biotin. Biotin is a natural compound that tightly binds proteins such as avidin or streptavidin. A compound labeled with biotin is said to be ‘biotinylated’. Biotinylated compounds can be detected with avidin or streptavidin when that avidin or streptavidin is conjugated another label such as a fluorescent, enzymatic, radioactive or other label. Similarly, a compound can be labeled with avidin or streptavidin and detected with a biotinylated compound.

Sample: A sample, such as a biological sample, is a sample obtained from a plant or animal subject. As used herein, biological samples include all clinical samples useful for detection via IHC including cells, tissues, and bodily fluids, including tissues that are, for example, unfixed, frozen, fixed in formalin and/or embedded in paraffin. In particular embodiments, the biological sample is obtained from a subject, such as in the form of a tissue biopsy obtained from a subject with a tumor. In other particular embodiments the sample can be a tissue section that is affixed to a microscope slide such as a glass microscope slide.

Specific binding: An association between two substances or molecules such as the association of an antibody with a polypeptide. As disclosed here, the antibody has specificity for the polypeptide (for example, a tissue antigen) to the significant exclusion of other, particularly similar polypeptides. Specific binding can be detected by any procedure known to one skilled in the art, such as by physical or functional properties. Specific binding can also be detected by visualization of a label (such as an enzymatic label) conjugated to, for example, the antibody molecule. In particular embodiments, specific binding includes binding with a dissociation constant (1(D) of 10⁻⁵ M or less, 10⁻⁸ M or less, 10⁻¹⁰ M or less, to 10⁻¹³ M or less. In particular embodiments, specific binding further includes binding to non-target antigens with a dissociation constant (KD) of 10⁻⁴M or more, in particular embodiments, of from 10⁻⁴M to 1 M or more.

Subject: A living multicellular vertebrate organism, a category that includes, for example, mammals and birds. A “mammal” includes both human and non-human mammals, such as mice. In some examples, a subject is a patient, such as a patient diagnosed with cancer, including a solid tumor cancer.

### II. Multiplex IHC

Disclosed are methods of performing multiplexed IHC on a FFPE tissue sample, preferably FFPE tissue sample that is affixed to a microscope slide such as a glass microscope slide. The methods involve contacting the sample with a first tissue antigen specific antibody. The tissue antigen specific antibody can be any antibody, such as a polyclonal antibody or monoclonal antibody (or any fragment thereof) that specifically binds to an antigen within tissue (which can be termed herein a ‘tissue antigen’). The antigen can be any antigen within a tissue including an antigen used to identify the cell as being of a particular type, a tumor antigen, an antigen expressed by a tumor bed or other stromal tissue, or any other antigen that is expressed on or within a cell to which an antibody response can be raised. The tissue antigen specific antibody can be labeled or unlabeled.

In further examples of the disclosed methods, the sample is contacted with a first labeled antibody that specifically binds the first tissue antigen specific antibody. Generally, the first labeled antibody is an antibody that specifically binds to antibodies of the particular immunoglobulin subtype and species from which the tissue specific antibody is derived. For example, if the tissue specific antibody is a rabbit polyclonal IgG that specifically binds to human CD8, then the labeled antibody can be any antibody that binds to rabbit IgG such as a mouse monoclonal antibody specific for rabbit IgG. The labeled antibody also includes a label. In some examples, the label includes an enzyme.

The sample is then contacted with a colorimetric substrate of the enzyme label such that when the substrate is acted upon by the enzyme, the substrate changes color, preferably from an undetectable color to a detectable color. Examples of enzyme labels include horseradish peroxidase, alkaline phosphatase, glucose oxidase, and β-galactosidase. Colorimetric substrates for horseradish peroxidase include ABTS (2,2′-azino-bis(3-ethylbenzothiazoline-6-sulphonic acid)), OPD (o-phenylenediamine dihydrochloride), TMB (tetramethylbenzidine), 4CN (4-chloro-1-napthol), DAB (3,3′-diaminobenzidine), and AEC (3-amino-9-ethylcarbazole). Colorimetric substrates for alkaline phosphatase include BCIP (5-bromo-4-chloro-3-indolyl-phosphate), and NBT (nitro-blue tetrazolium chloride)—often used together. Colorimetric substrates for glucose oxidase include NBT. Colorimetric substrates for β-galactosidase include X-Gal (5-bromo-4-chloro-3-indolyl-β-D-galactopyranoside). The colorimetric substrate's reaction with the enzyme label of the first labeled antibody results in the visualization of one or more cells that express the tissue antigen. In some examples, the tissue specific antibody is labeled, for example, with an enzyme label. In such a case, the tissue specific antibody and the labeled antibody are the same reagent.

In further examples of the disclosed methods, a digital image of one or more cells that express the first tissue antigen is generated. The digital image can be generated using any of a number of methods and/or devices including the use of a bright field, fluorescent or other microscope equipped with a camera that can capture a digital image of the cells within the context of the tissue and with digital storage capabilities (within the camera or in another device) that can save the image.

In still further examples, the colorimetric substrate is destained by any appropriate process including by washing in an alcohol solution.

In still further examples, the tissue antigen specific antibody and the enzyme labeled antibody are removed by heating the sample to at least 90° C. in a buffer solution for a sufficient time to remove the tissue specific antibody and the labeled antibody. While one of skill in the art can readily determine without undue experimentation the length of time required to remove the tissue specific and labeled antibody, in some examples, the samples are maintained at the temperature of at least 90° C. for at least 15 minutes. In some examples, the sample and the slide to which it is affixed are immersed in the buffer solution and the sample and buffer solution heated in a standard microwave oven. In other examples, the samples and slides are immersed in a buffer solution preheated to 90° C. using a hot plate, kettle, or other heating element. In other examples, the preheated buffer solution is added to a container that encloses the samples and slides, such as by an automated methodology. In some examples, the buffer is a citrate buffer. In still further examples, heating the sample occurs after contacting the sample with the tissue specific antibody, labeled antibody, and colorimetric substrate and after the digital image is generated. In other examples, the heating of the sample occurs between the first and second staining cycles.

As disclosed herein, the process of (a) contacting the sample with the first tissue specific antibody; (b) contacting the sample with the first enzyme labeled antibody, (c) contacting the sample with a colorimetric substrate of the enzyme labelled antibody, (d) generating the first digital image, (e) destaining the colorimetric substrate, and (f) removing the tissue antigen specific antibody and first labeled antibody with a microwave heat treatment is termed a staining cycle. In further examples, a second staining cycle, third staining cycle, fourth staining cycle, fifth staining cycle, sixth staining cycle, and seventh staining cycle are completed. In still further examples, an eighth, ninth, tenth, eleventh, and twelfth staining cycle are completed. In still further examples a thirteenth, fourteenth, and fifteenth staining cycle are completed. In still further examples, sixteen, seventeen, eighteen, nineteen, or twenty or more than twenty staining cycles are completed. In particular embodiments 36-60 staining cycles are completed. In particular embodiments, in each staining cycle, a digital image is captured after contacting the sample with the colorimetric substrate. In some examples, a different tissue antigen is used. In other examples, one or more of the tissue antigens can be used in more than one staining cycle. In all but the last staining cycle, the slides are heat treated to remove the tissue specific and enzyme labeled antibodies. In the last staining cycle, the heat treatment is optional.

In some examples, the tissue is subjected to antigen retrieval methodologies prior to the contacting with the tissue specific antibody. Formalin fixed tissues, while preserving the tissue, can cause antigens in the tissue to be rendered ‘masked’ to tissue antigen antibodies due to amino acid crosslinking within the tissue, conformational changes of the antigen, or other factors. Antigen retrieval methodologies can involve treatment with heat, treatment with a solution (such as an acidic or basic solution), treatment with an enzyme (such as a proteinase), any combination thereof or any other methods of antigen retrieval known in the art.

In other examples the tissue is stained with stains that allow visualization of cellular structures such as the cytoplasm, nucleus and cell membrane. Such stains include hematoxylin, eosin, periodic acid-Schiff's stain, Mason's Trichrome, Gomori Trichrome, silver salts, Wright's, Giemsa, and others. A digital image of the stained tissue, termed herein a structure-stained digital image, is generated and can be used in further processing such as coregistration and cell segmentation.

In some examples, the digital images collected from the staining cycles are coregistered such that they can be merged into a composite digital image. In still further examples, expression of each tissue antigen is given a different color in the composite digital image such that the expression of different cell types within the sample can be visualized. In still further examples, the expression of each tissue antigen is quantified by, for example, performing cell segmentation using, for example, a watershed segmentation algorithm. In still other examples, expression of a particular tissue specific antigen can be quantified using the digital images.

In still other examples, a panel of tissue specific antibodies is used to label the sample in succession. The panel can include seven, eight, nine, ten, eleven, twelve, thirteen, fourteen, fifteen, sixteen, seventeen, eighteen, nineteen, twenty, thirty, forty, fifty, sixty, or more than sixty tissue specific antibodies. Antibodies in the panel can be selected for any of a number of purposes. In some examples, the tissue specific antibodies in the panel label tissue specific antigens that identify cells as particular immune cells. In other examples, the tissue specific antibodies in the panel label tumor antigens.

In addition to the lymphoid and myeloid biomarker panels described herein, further combinations of 12-biomarker panels can be developed, enabling more detailed immune profile studies, other TME characteristics including neoplastic cell, vascular and/or mesenchymal support cell phenotypes, among others. Tumor molecular subtypes of human breast, pancreas, lung, and other cancers, can be developed. Such panels can also include markers of vascular characteristics as such markers indicate the effects of leukocyte infiltration and emigration in and out of tissues and tumors.

### EXAMPLES

The following examples are for illustration only. In light of this disclosure, those of skill in the art will recognize that variations of these examples and other examples of the disclosed invention be possible without undue experimentation.

### Example 1

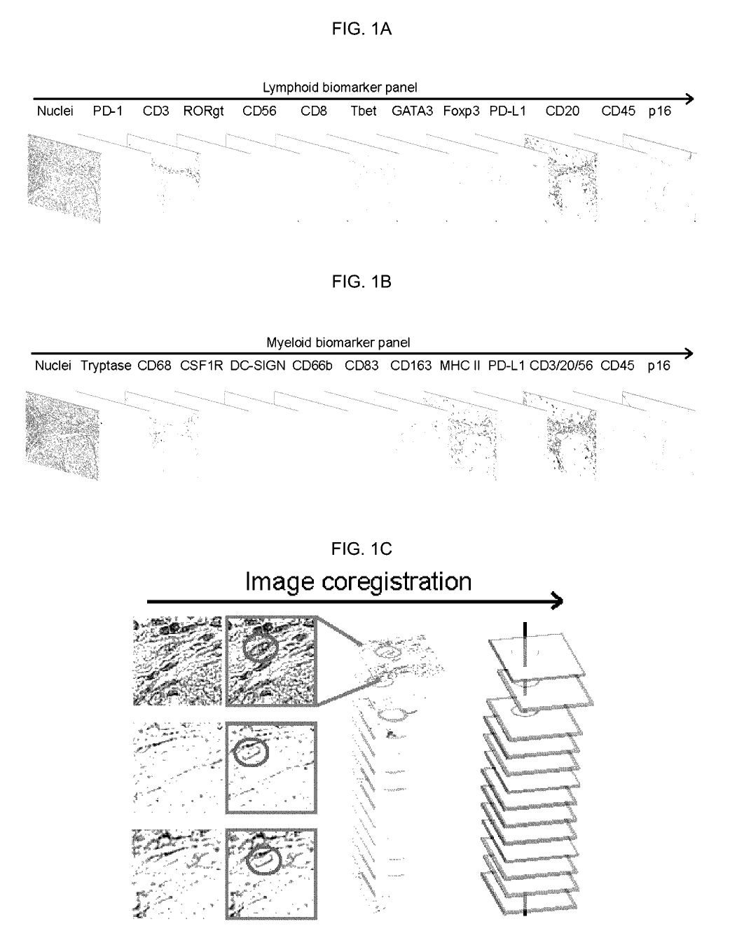

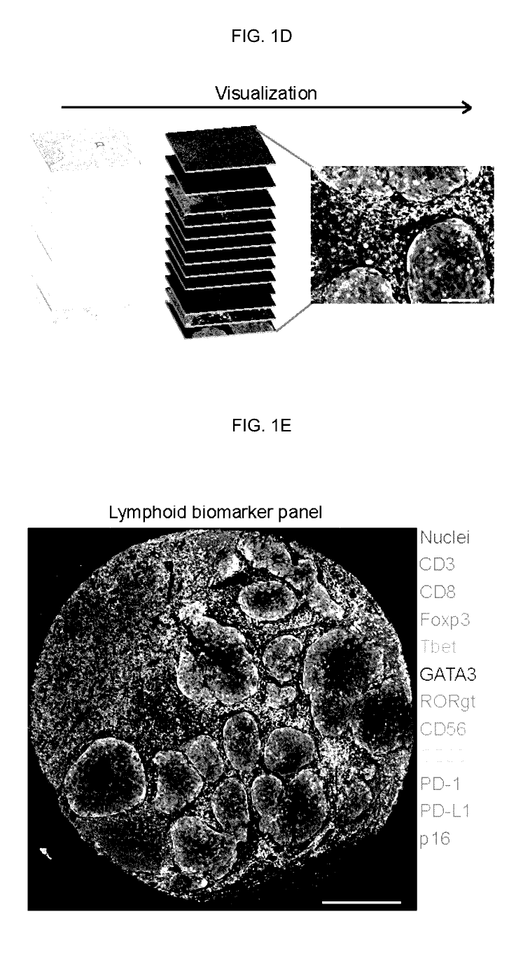

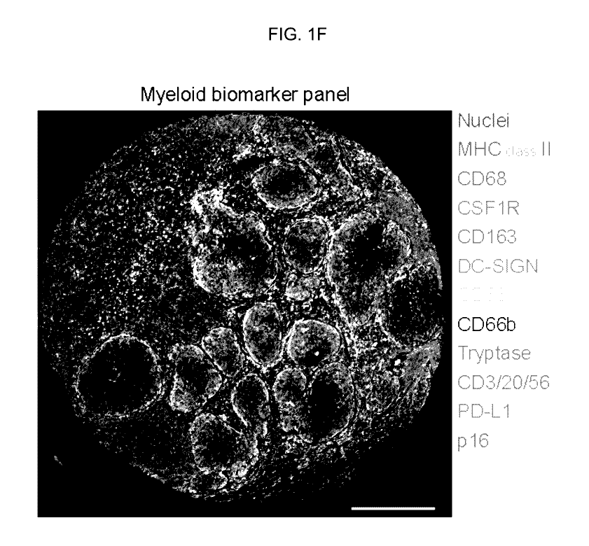

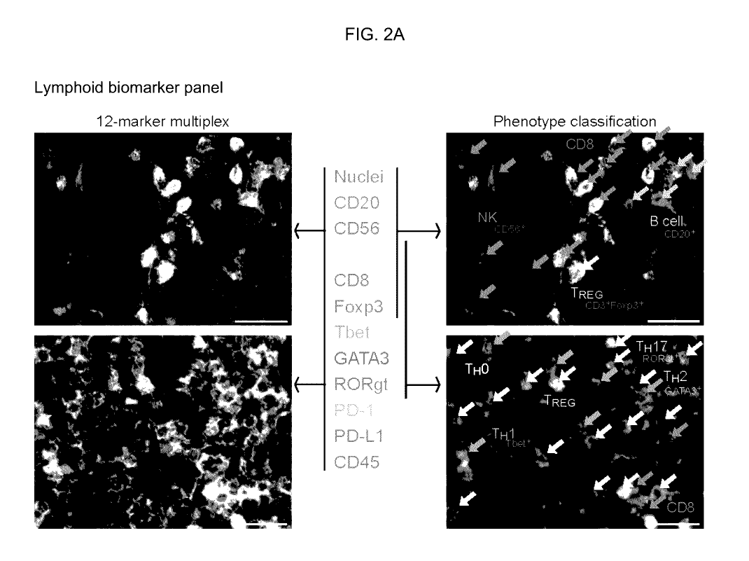

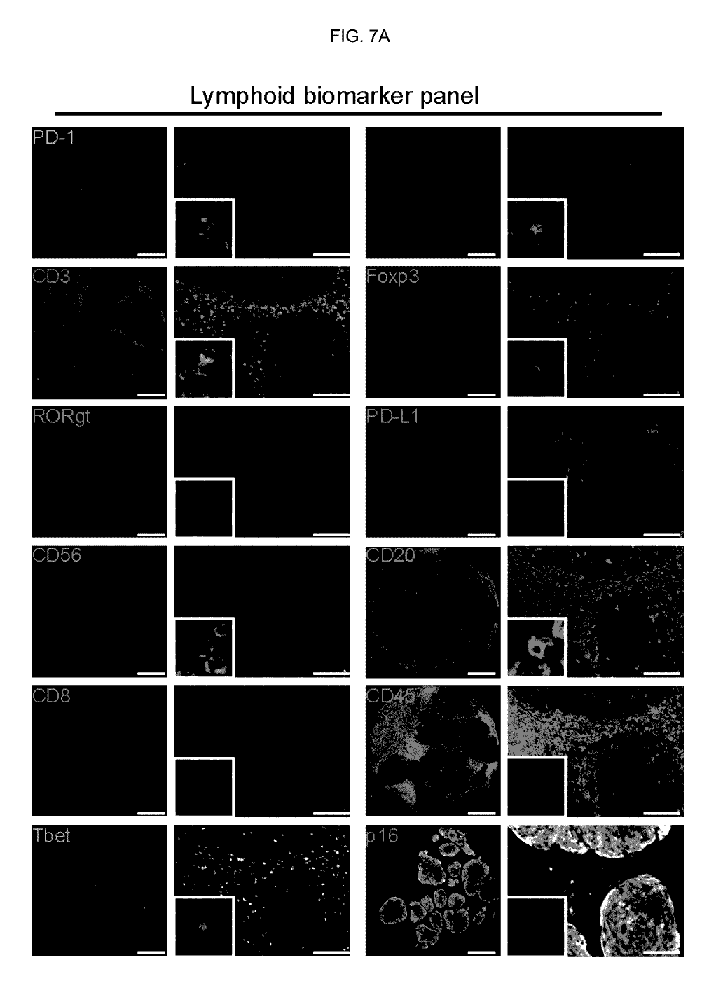

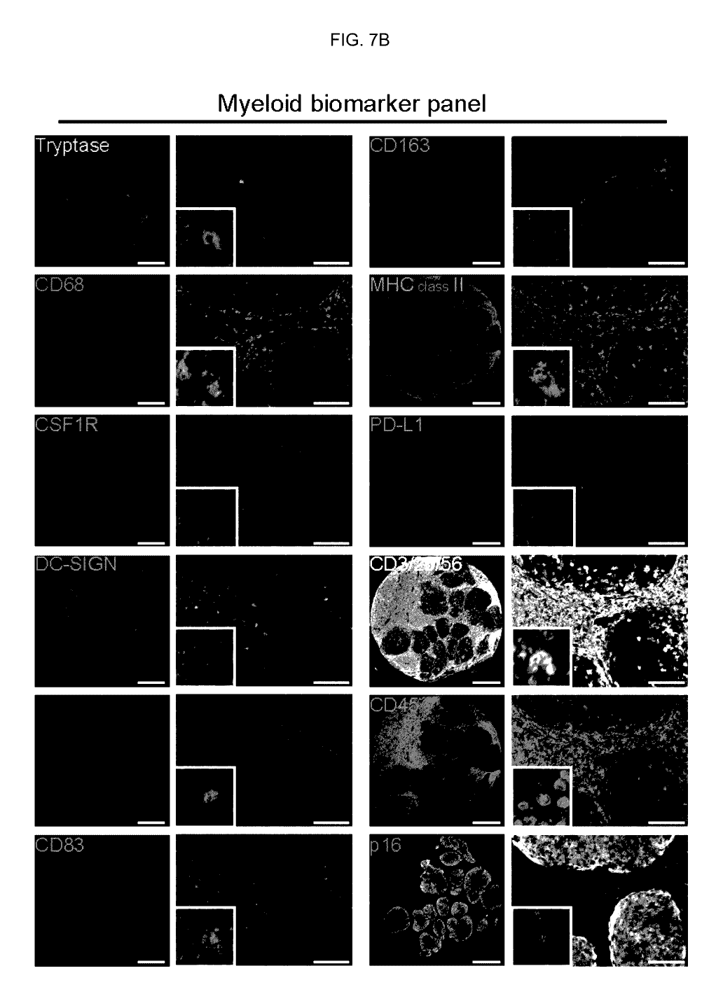

Immunodetection of 12 biomarkers in one FFPE tissue section. Given the practical limitations of conventional IHC approaches, an IHC workflow enabling simultaneous evaluation of 12 immune-based biomarkers in FFPE sections was developed. A sequential IHC methodology originally reported for a 5-plex protocol (Glass et al, J Histochem Cytochem 57, 899-905 (2009); incorporated by reference herein) served as a template. This methodology was improved upon and the resulting methods allowed analysis of more than 10 different proteins in a single tissue section, with no limitations on the species of origin for detecting antibodies (FIGS. 1A, 1B, 7A and 7B).

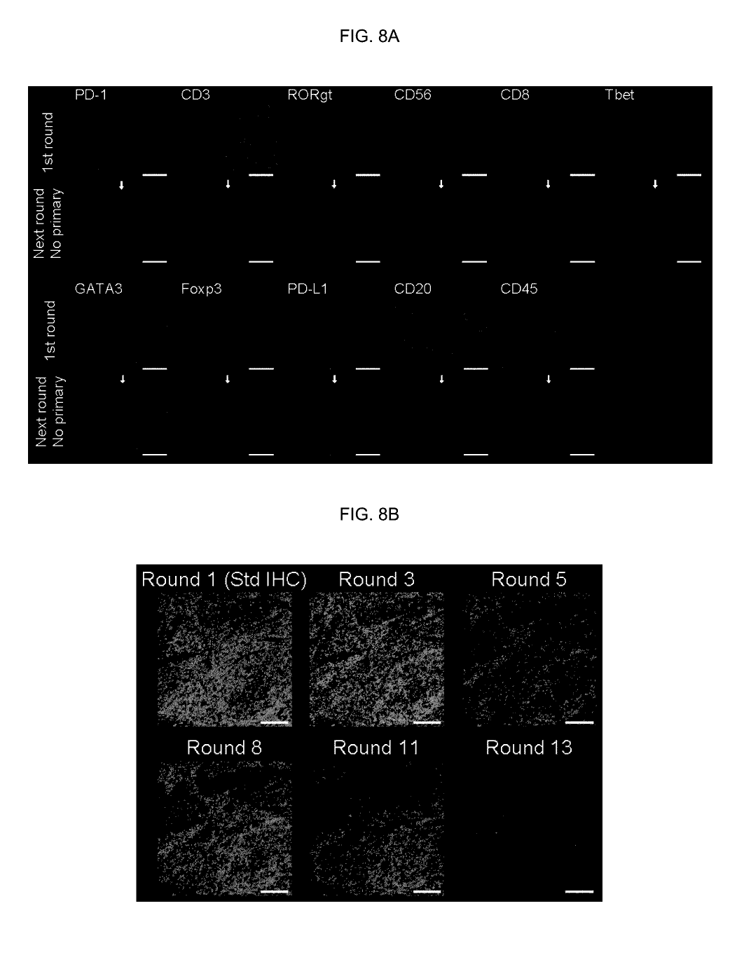

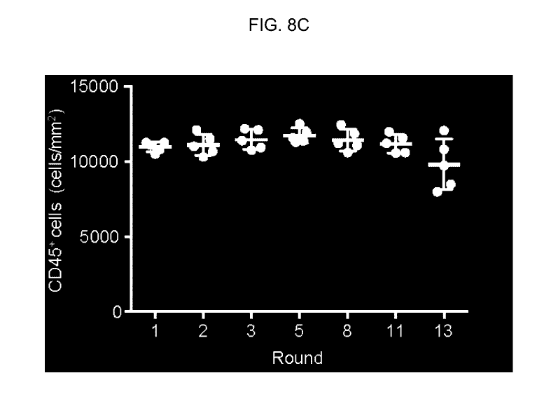

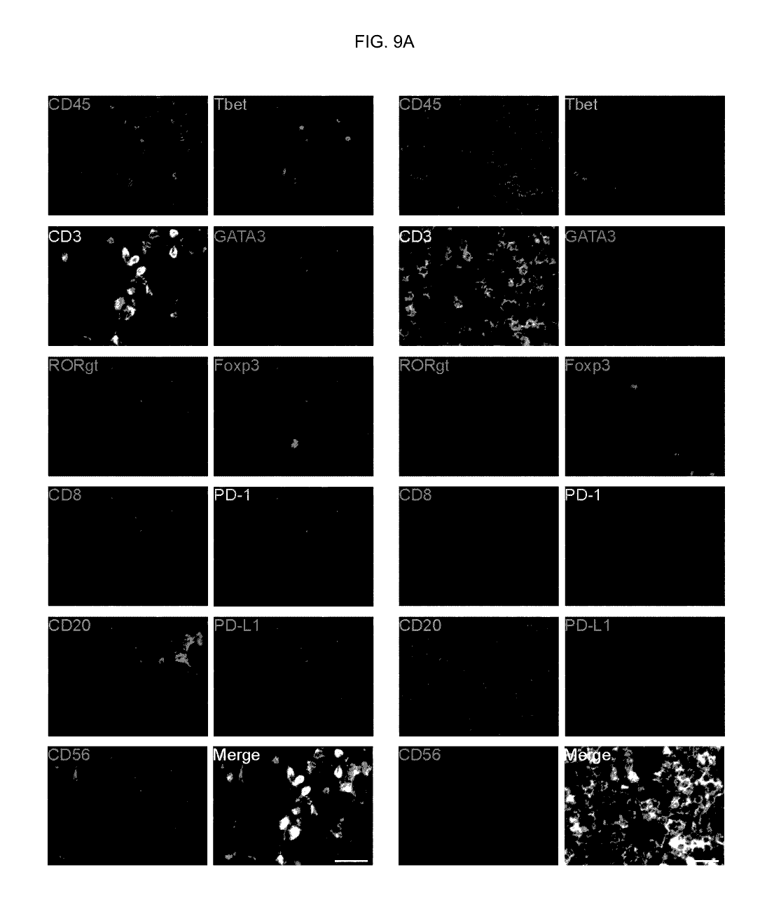

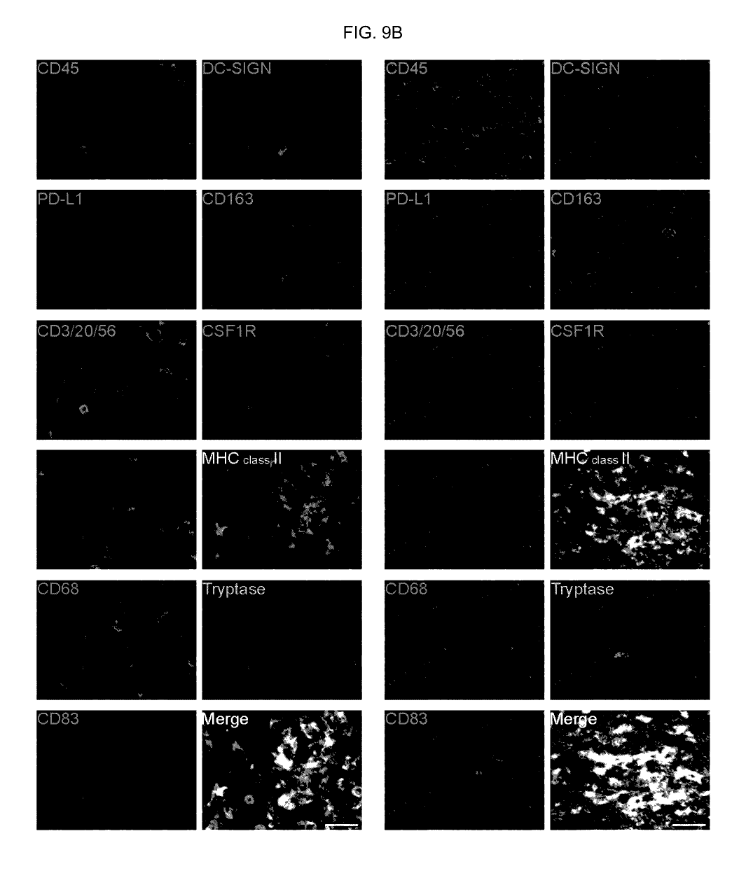

After standard IHC preparation and primary antibody incubation, antibodies are detected by a F(ab′) fragment-specific secondary antibody labeled polymer-based peroxidase. Following detection, slides are visualized by alcohol-soluble peroxidase substrate 3-amino-9-ethylcarbazole (AEC), followed by whole tissue digital scanning. Iterative staining is achieved by AEC washing slides in ethanol (Tramu et al, J Histochem Cytochem 26, 322-324 (1978); incorporated by reference herein), followed by antibody stripping in a volume of heated citrate buffer (pH 6.0) (Lan et al, J Histochem Cytochem 43, 97-102 (1995); incorporated by reference herein). Slides are then washed and equilibrated in binding buffer, and readied for a subsequent round of primary antibody incubation, and sequential digital imaging. After completing multiple rounds of sequential IHC, serially scanned and digitized images are aligned based on semi-automated coregistration pipeline utilizes CellProfiler software as a backbone (Carpenter et al, Genome Biol 7, R100 (2006); incorporated by reference herein) (FIG. 1C). Coregistered images are subsequently transferred to ImageJ (Schneider et al, Nature Methods 9, 671-675 (2012); incorporated by reference herein), and AEC/hematoxylin-color information is extracted by color deconvolution algorithms (Ruifrok et al, Immunohistochem Mol Morphol 11, 85-91 (2003); incorporated by reference herein), wherein images are converted to gray scale, and then colorized by pseudo-immunofluorescence (FIGS. 1D, 7A, and 7B). Complete stripping of antibodies and signals throughout all cycles was confirmed (FIG. 8A). IHC sensitivity was equivalent to standard IHC throughout 11-repeated antibody-stripping rounds (FIGS. 8B and 8C). Following sequential staining and image processing, multicolor composite images are generated by overlying each pseudo-colored image (FIGS. 1E and 1F).

### Example 2

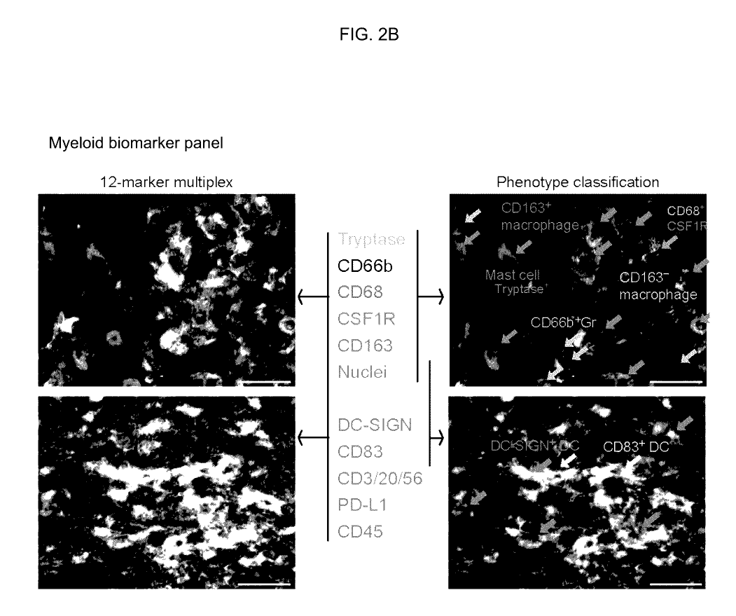

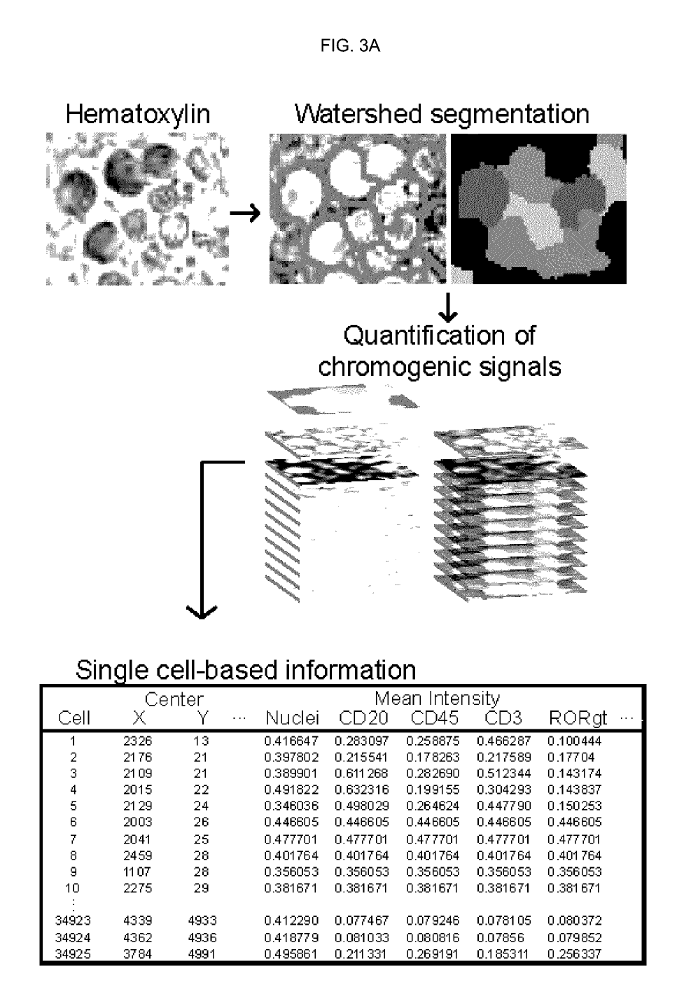

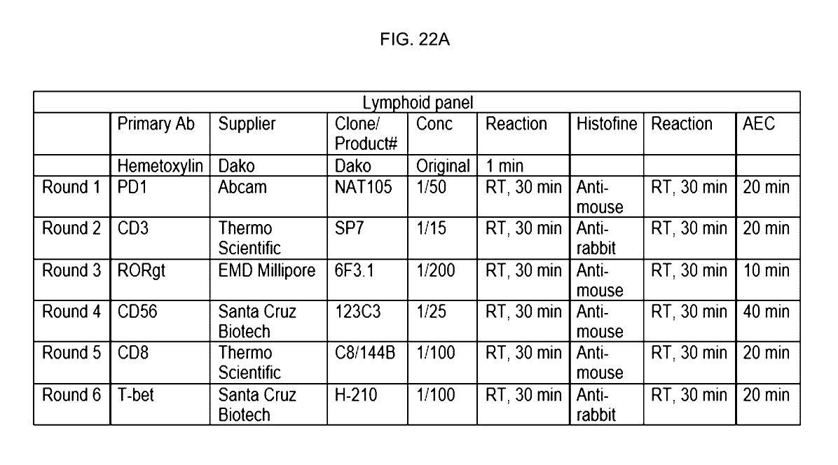

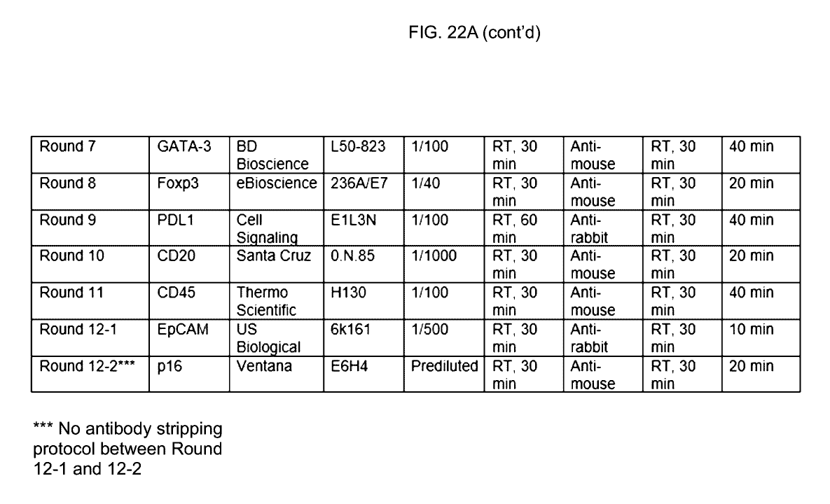

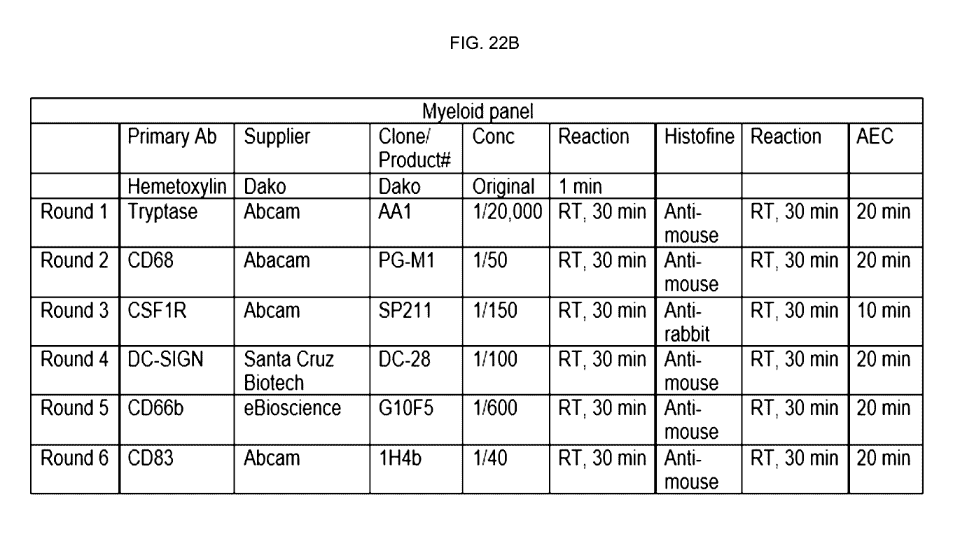

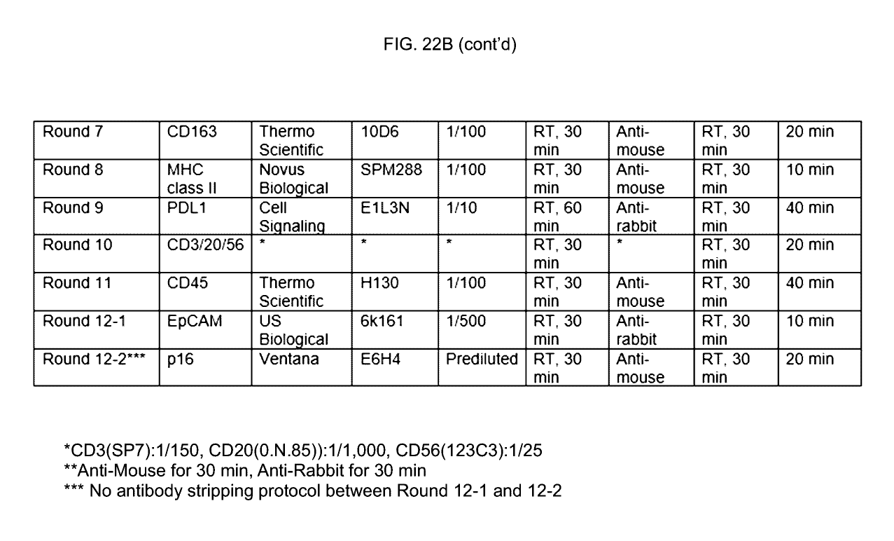

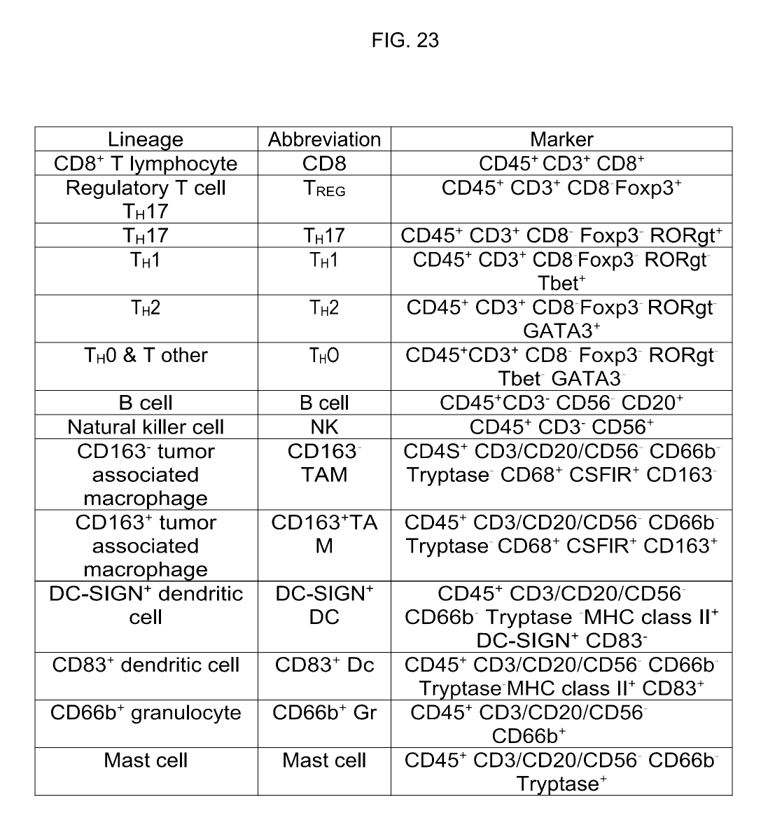

Two 12-panels of lineage-selective antibodies phenotype lymphoid and myeloid cells. Since multiplex capability enables colocalization of multiple biomarkers in single cells, two panels of 12-biomarkers apiece, encompassing 19-distinct epitopes were established to phenotype lymphoid and myeloid cell lineages as indicated in FIGS. 22A-22B and FIGS. 1E and 1F. By utilizing two serial FFPE sections and the lymphoid and myeloid biomarker panels, expression and colocalization of multiple biomarkers identify immune cell lineages including CD8+ T cell (CD8), TH0, TH1, TH2, TH17, Regulatory T cell (TREG), B cell, natural killer cell (NK), CD163− tumor-associated macrophage (TAM), CD163+ TAM, DC-SIGN+ immature dendritic cell (DC), CD83+ mature DC, CD66b+ granulocyte (Gr) including neutrophils and eosinophils, and mast cells (FIGS. 2A, 2B, 8A, 8B, 8C, and 8D) based on lineage markers indicated in FIG. 23. Human surgical specimens reflecting pancreatic ductal adenocarcinoma, breast adenocarcinoma, and malignant mesothelioma (N=34, 6, and 22, respectively) were analyzed, as well as murine tissues/tumors, revealing the broad applicability of the approach (FIGS. 22A and 22B).

### Example 3

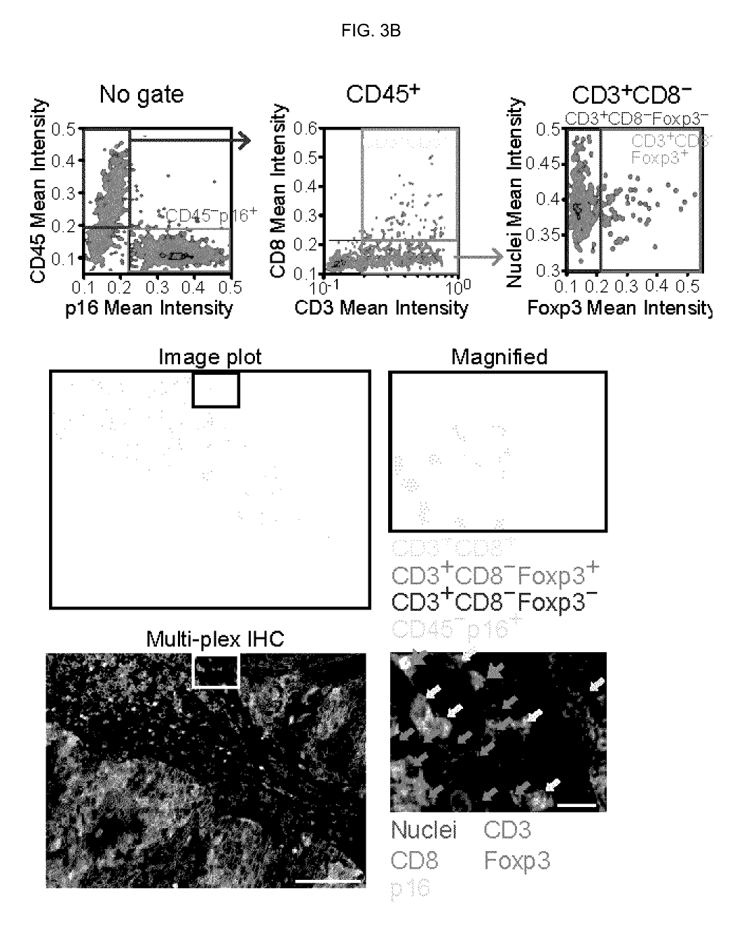

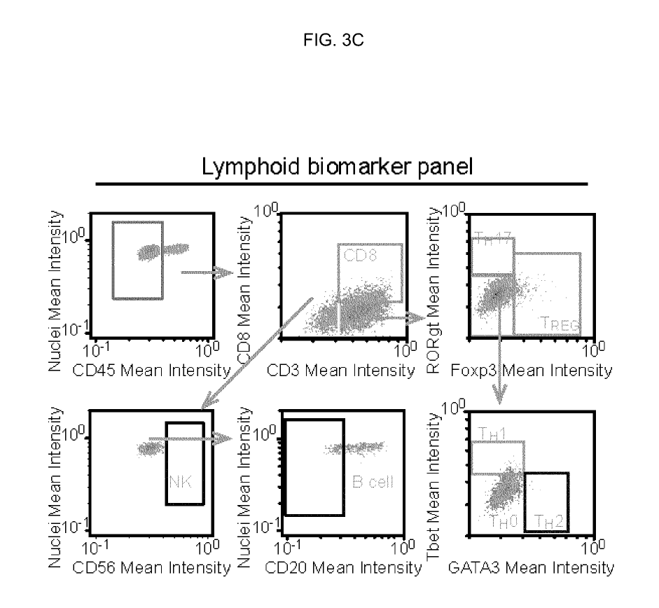

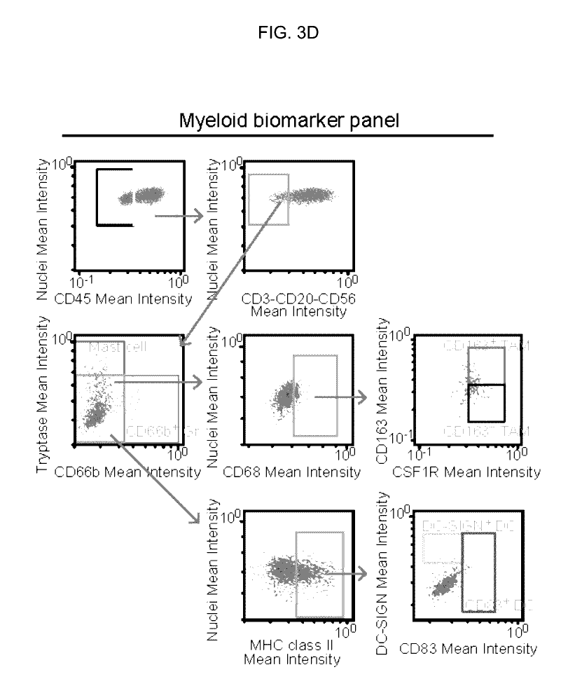

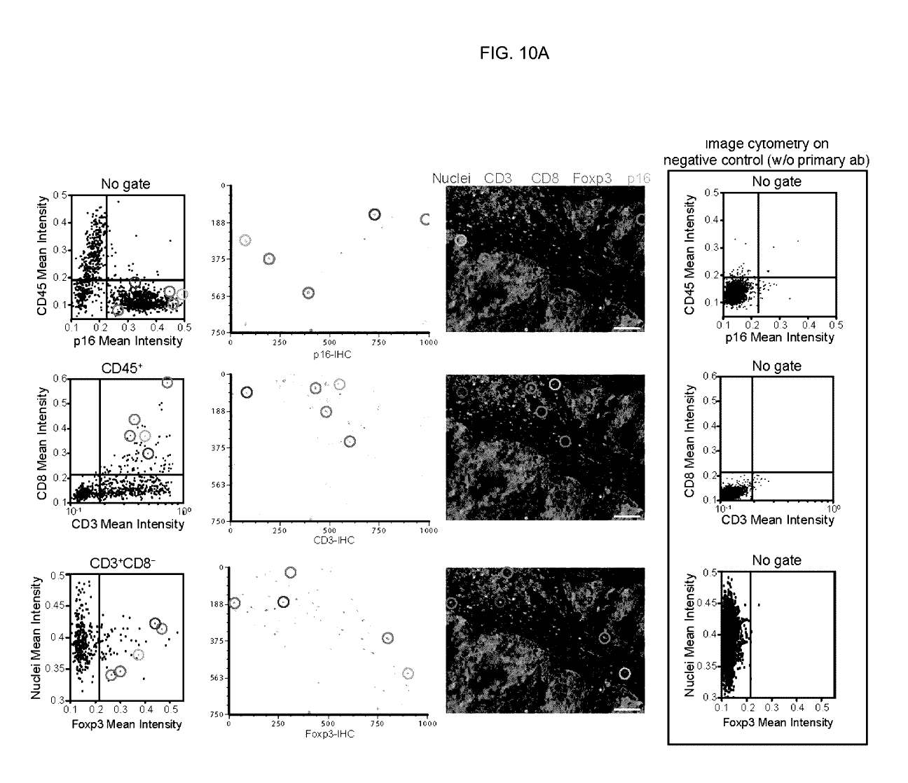

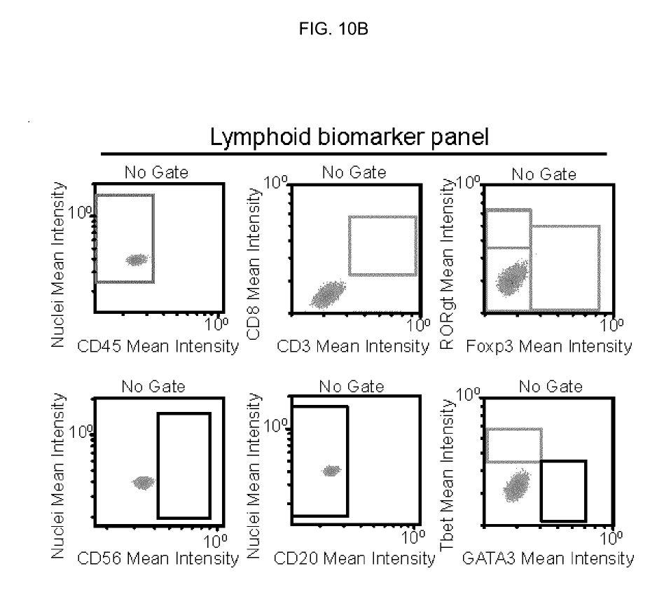

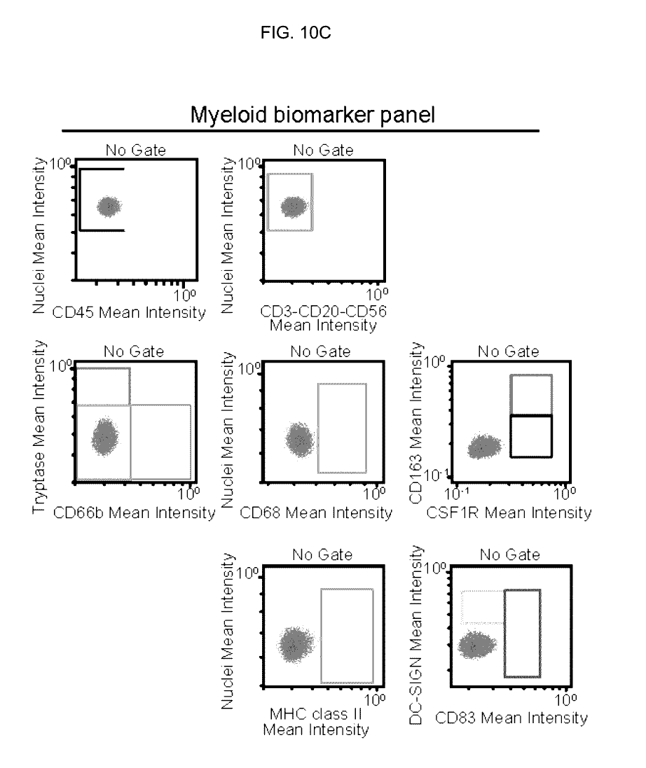

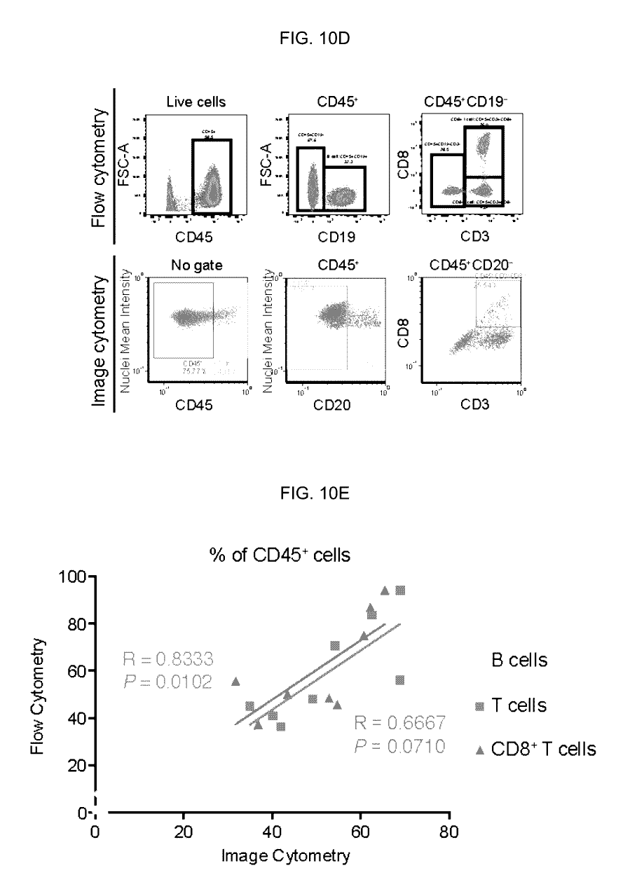

Multiparameter image cytometry analysis of 16-different cell lineages. A multiparameter cytometric quantification method was established to quantify multiplexed images with regional and proximity analytics. The method was developed via evaluation of single cell-based chromogenic intensities based on single cell segmentation algorithms, using CellProfiler. Hematoxylin-stained images for cell segmentation based on watershed segmentation algorithms (Wahlby et al, J Microsc 215, 67-76 (2004) and Padmanabhan et al, J Neurosci Methods 193, 380-384 (2010); both of which are incorporated by reference herein) were used, followed by quantification of chromogenic signals in serial AEC-stained images, providing multiparametric information including cell size, compactness, and location with chromogenic intensity for each protein biomarker (FIG. 3A). Single cell-based information, including the pixel intensity and shape-size measurements was visualized and analyzed with qualitative assessment of signal intensities, analogous to FACS (FIG. 3B). Thresholds for qualitative identification were determined based on distribution of plots for each marker in negative control slides (FIG. 10A). Gated cells in dot plots were visualized in the original image together with distribution in the tissue context (FIG. 3C). XY coordinates of selected single cells were also depicted in the original image, enabling positioning of each cell in the corresponding image (FIG. 10A).

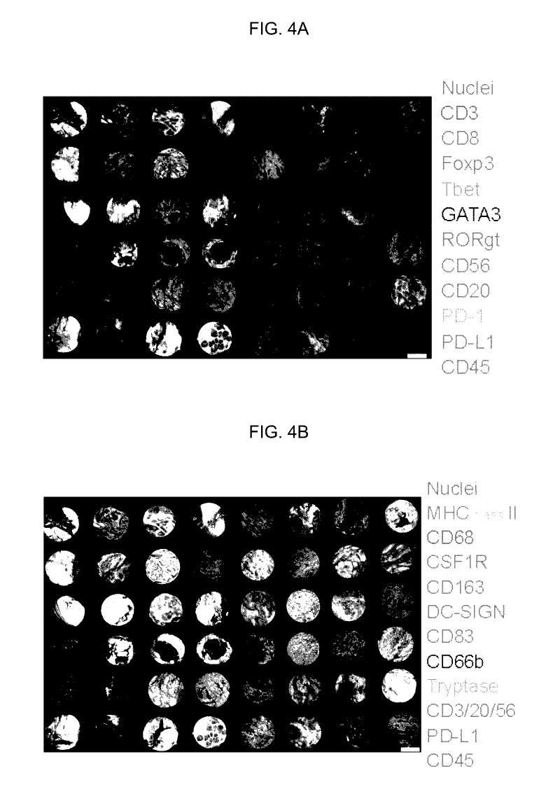

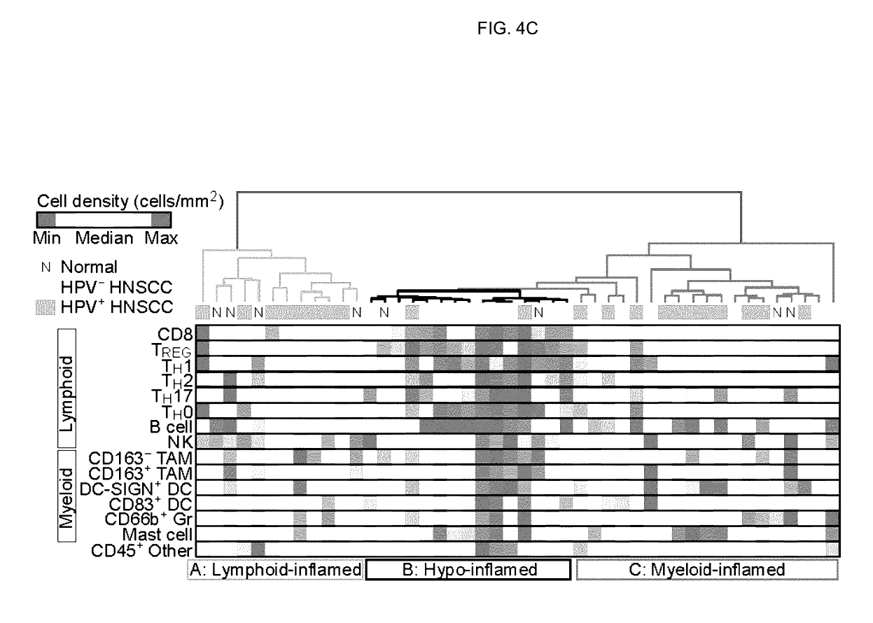

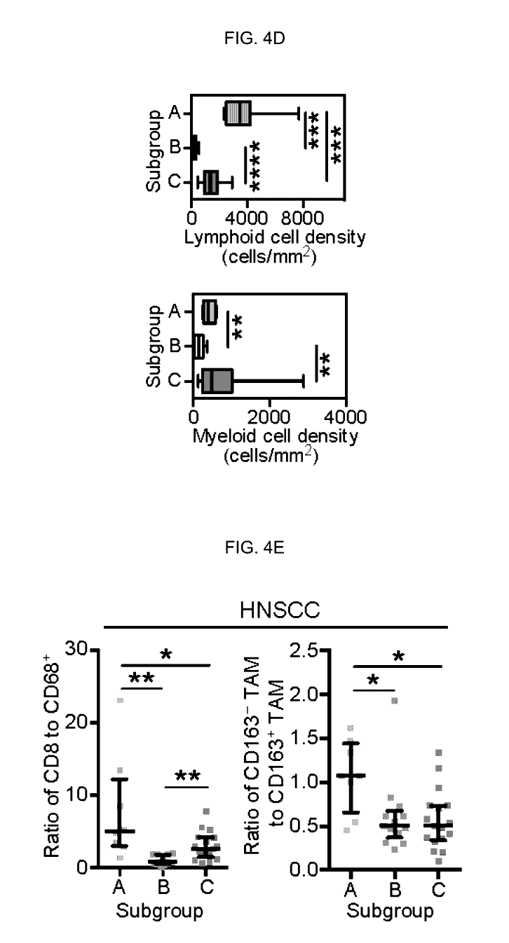

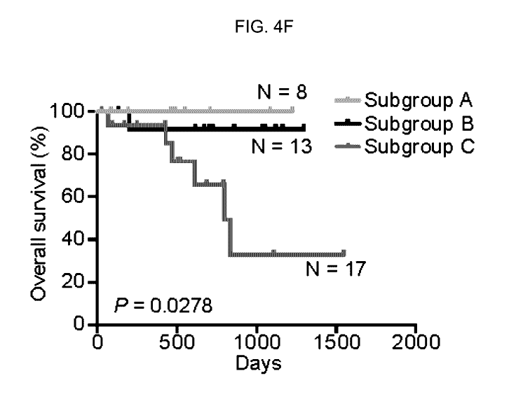

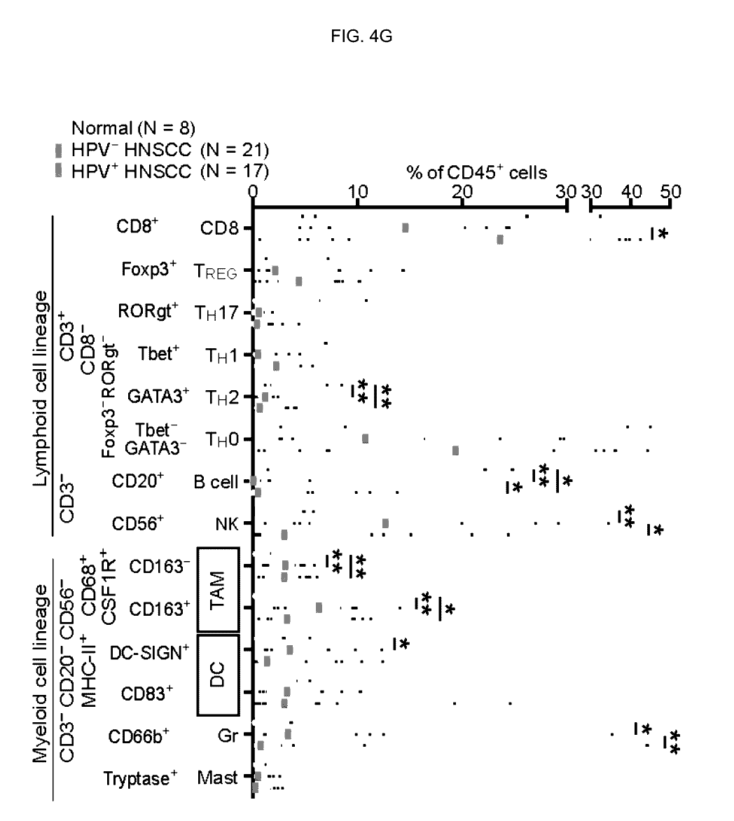

To achieve quantitative data analogous to multiparametric 12-color FACS, qualitative gating strategies were developed for the two panels that bind the lineage biomarkers indicated in FIGS. 22A-22B and FIGS. 3D, 3D, 10B, and 100. For comparative analyses between image cytometry and FACS, the same pieces of human surgical specimens were divided into two pieces, and evaluated by single-cell suspension-based FACS analysis and FFPE section-based image cytometry. Positive correlations were observed in percentages of T and B cells measured by FACS and image cytometry (N=9, FIGS. 4D and 4E), validating the quantification based on the image cytometric approach, image cytometry.

### Example 4

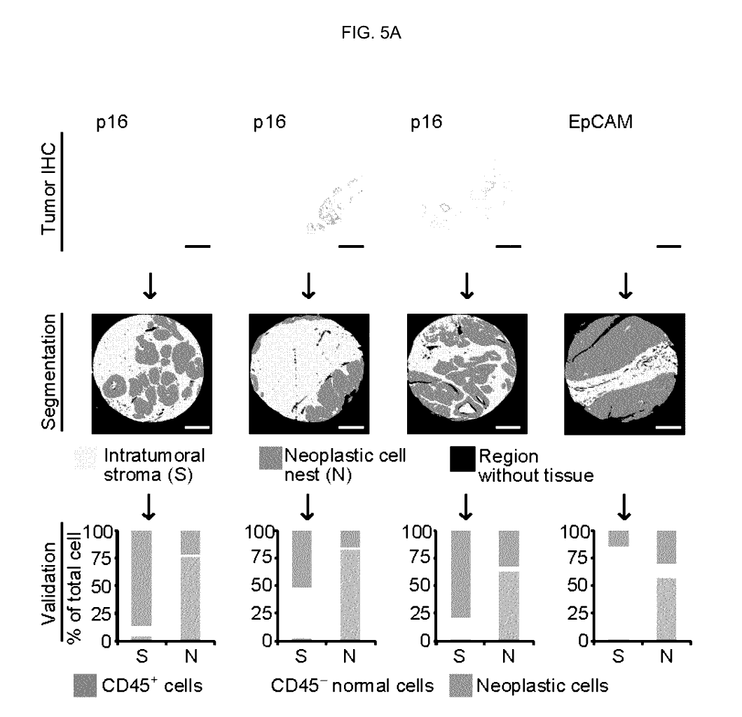

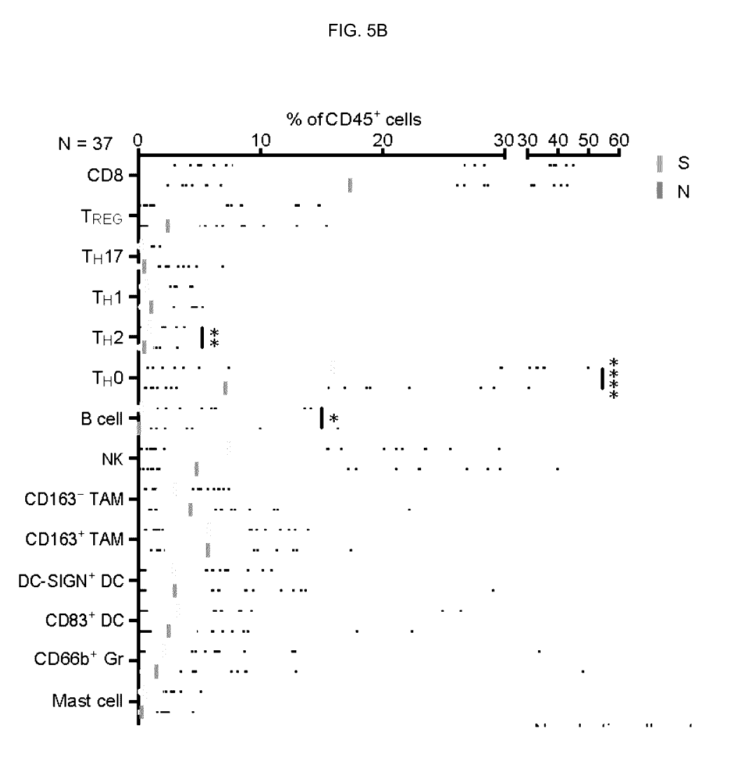

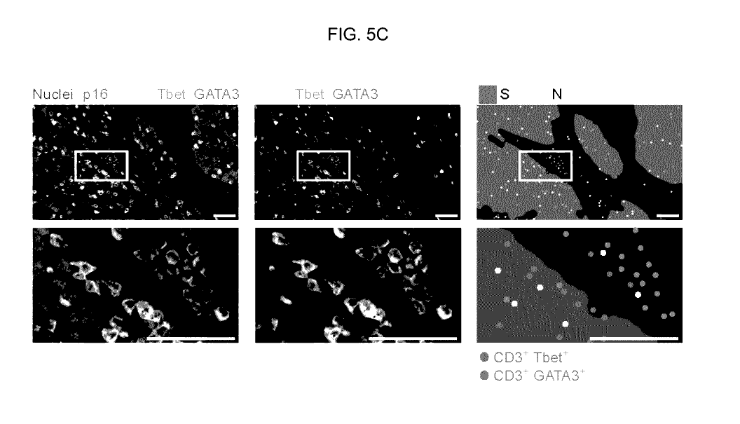

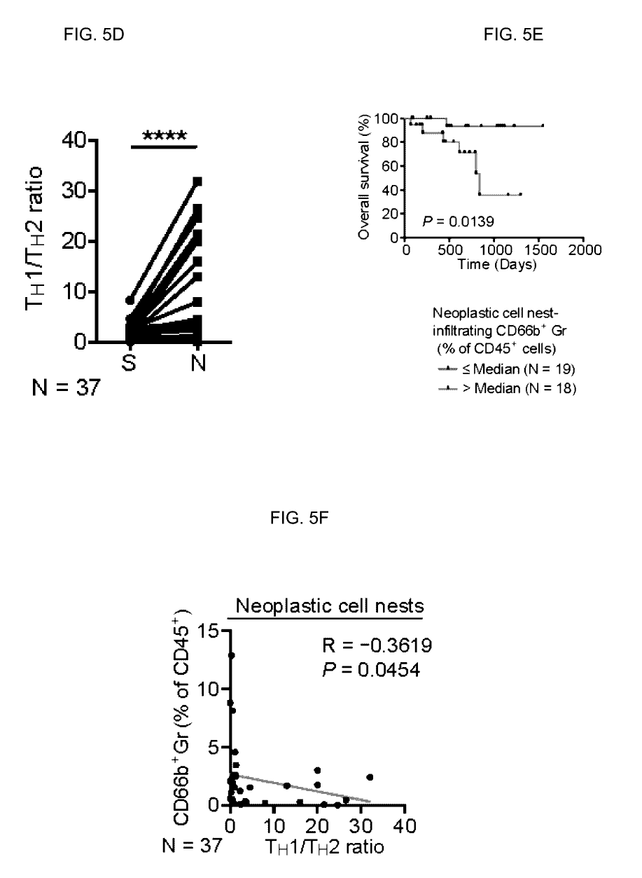

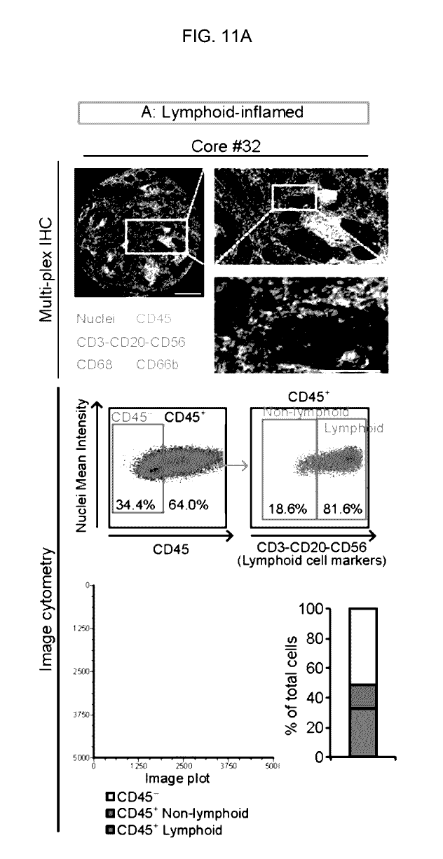

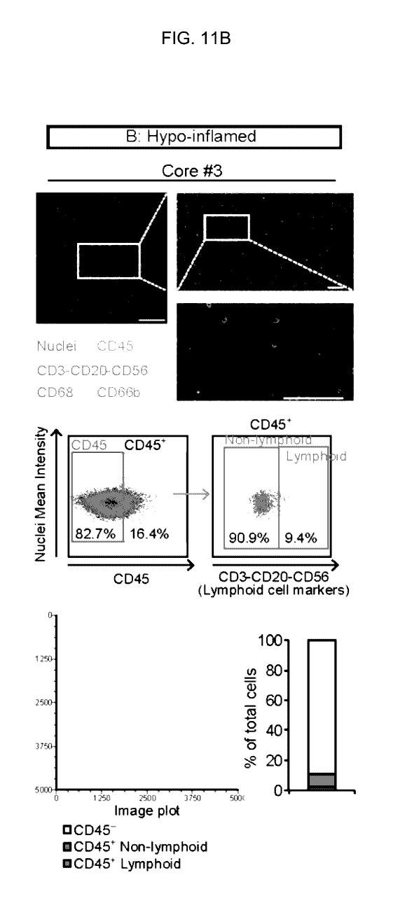

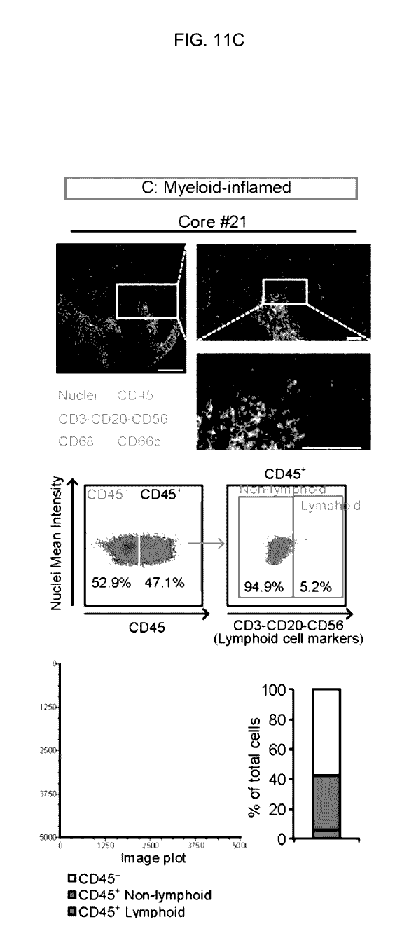

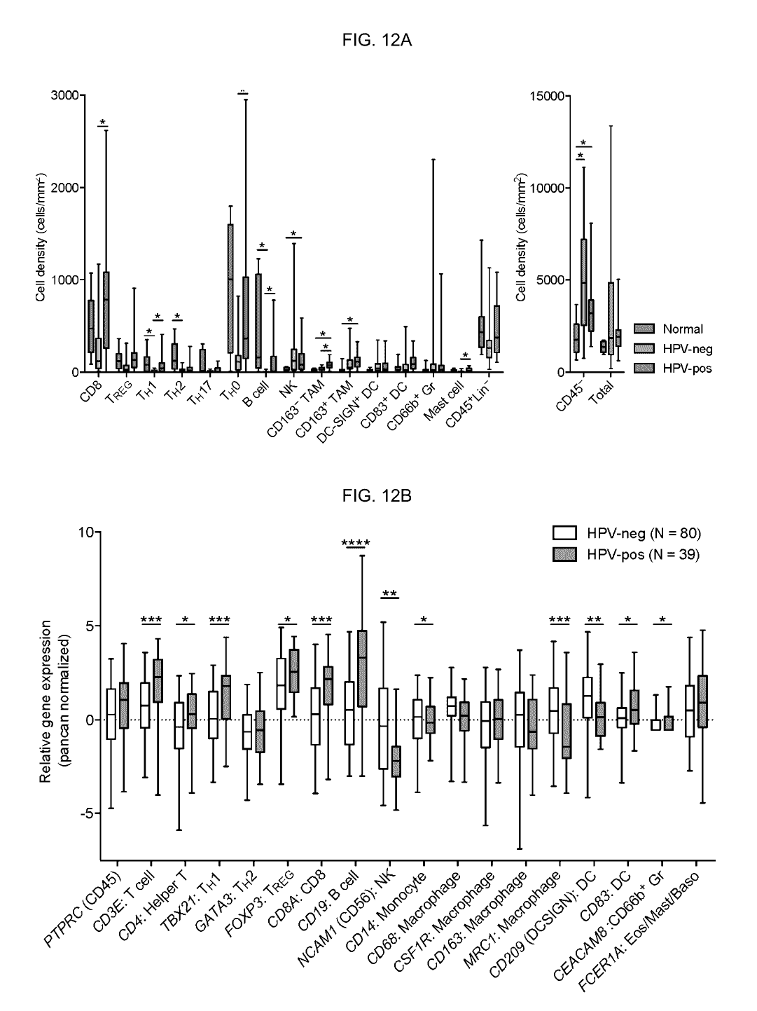

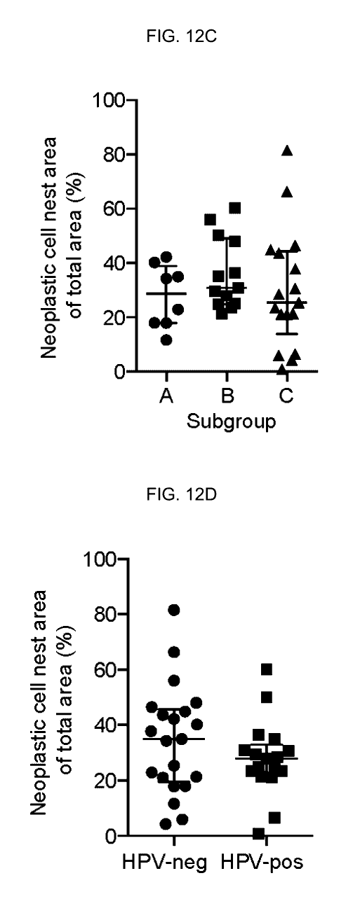

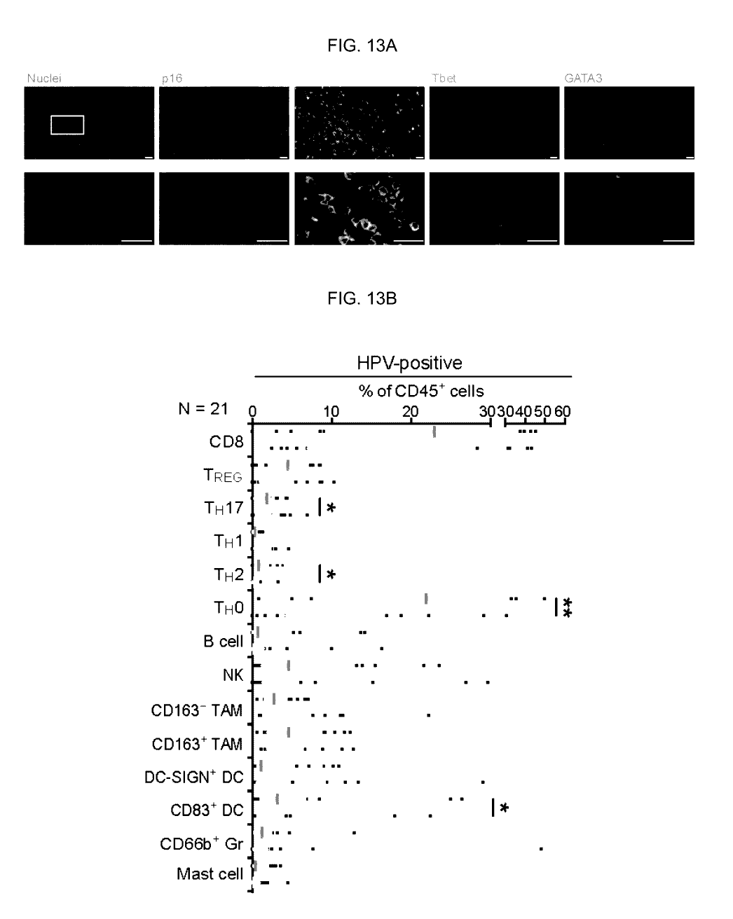

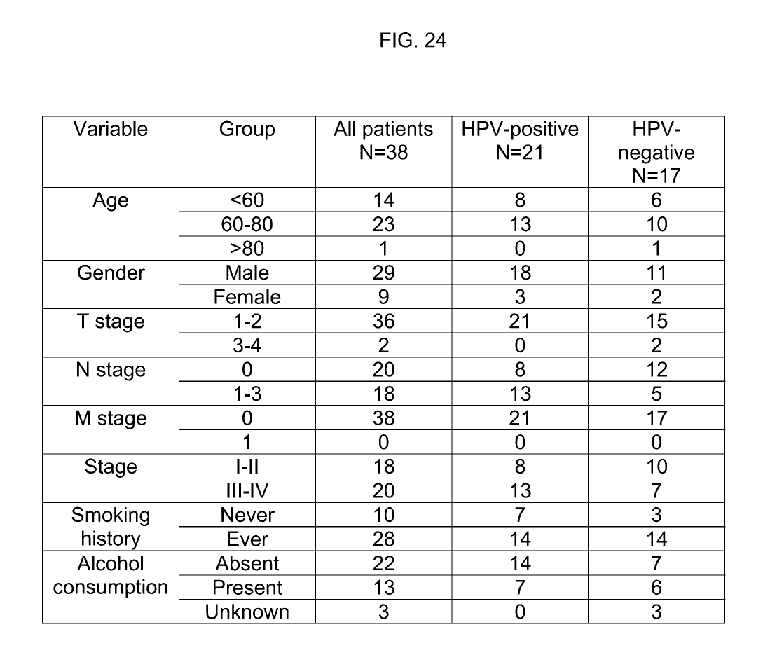

Immune cell density-based tumor subclassification correlating with tissue type and prognosis in head and neck squamous cell carcinoma (HNSCC). Immune-based biomarkers associated with clinical outcomes were also assessed. A tissue microarray (TMA) including oropharyngeal HNSCC tissues was assessed. In such tissues, the presence of oncogenic human papilloma virus (HPV) is associated with immunogenic gene signatures (Thurlow et al, J Clin Oncol 28, 2881-2888 (2010) and Keck et al, Clin Cancer Res 21, 870-881 (2015); both of which are incorporated by reference herein). The TMA was assembled from 2 mm cores derived from pathologist-selected representative intratumoral areas. These included 21 HPV-positive cancer tissues, 17 HPV-negative cancer tissues, and 8 non-malignant pharyngeal tissues (FIG. 24). IHC using the lymphoid and myeloid panels described herein (FIGS. 22A and 22B) was performed, showing heterogeneous patterns of immune cell infiltration across the cohort (FIGS. 4A and 4B). Following evaluation of densities of cell lineages identified by gating strategies in image cytometry (FIGS. 3C and 3D), an unsupervised hierarchical clustering analysis was performed to identify potential distinct subgroups based on immune profiles. This analysis revealed presence of lymphoid-inflamed (A), hypo-inflamed (B) and myeloid-inflamed (C) subgroups, where lymphoid and myeloid lineage cells were differentially infiltrated (FIGS. 4C and 11). This observation was supported by cell density analyses among the three groups (FIG. 4D). Utilizing transversal quantification of multiple immune cell lineages, ratios of CD8/CD68 and CD163− TAM/CD163+ TAMs were evaluated as IHC-based biomarkers (previously reported as favorable predictors of clinical outcomes in other malignancies; Mantovani et al, Trends Immunol 23, 549-555 (2002); incorporated by reference herein). While the hypo-inflamed group unsurprisingly showed low scores reflecting a “cold” inflammatory status, both ratios in the lymphoid-inflamed subgroup appeared significantly higher than those in the myeloid-inflamed subgroup, (FIG. 4E). In comparison with this observation, the myeloid-inflamed subgroup exhibited the worst overall survival among three subgroups (FIG. 4F). A leukocyte composition analysis of total CD45+ demonstrated high CD163- and CD163+ TAMs, and low TH2 and B cells in HNSCC tissues in comparison to normal pharynx (FIG. 4G). HPV-positive status was associated with high CD8, while HPV-negative correlated with high NK and DC-SIGN+ DC, and CD66b+ Gr (FIG. 4G), together indicating presence of differential immune profiles between benign and malignant tissues as well as HPV-status. Distinct immune profiles depending on HPV-status were also confirmed by cell density-based analysis (FIG. 12A). These observations were further supported by The Cancer Genome Atlas (TCGA) analysis, revealing similar tendencies by comparison based on HPV-status (FIG. 12B). Tumor area as a percentage of total tissue in each core was also evaluated (see FIG. 5A) No significant differences between subgroups (FIGS. 12C and 12D) were observed, thereby excluding a potential bias from effects of differential tumor-stroma ratios on immune cell densities. Although the lymphoid- and hypo-inflamed subgroups were clearly associated with HPV-positive and -negative status respectively, the myeloid-inflamed subgroup with poor prognosis showed heterogeneity in HPV-status (FIG. 4C), indicating the possibility that further stratification of patients with HNSCC based on immune profiles beyond HPV-status may be warranted.

### Example 5

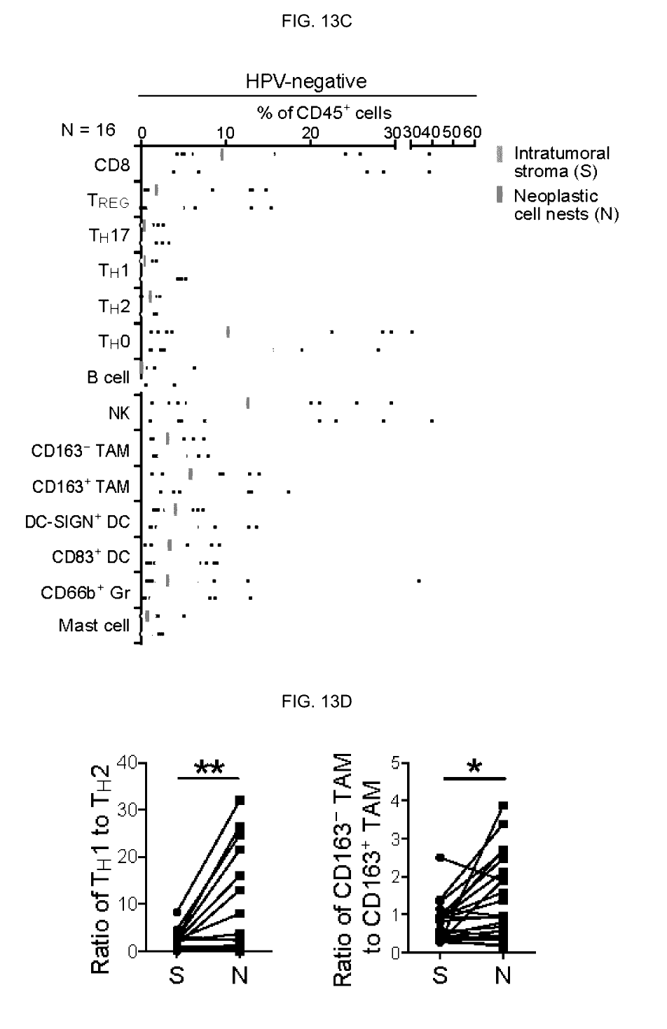

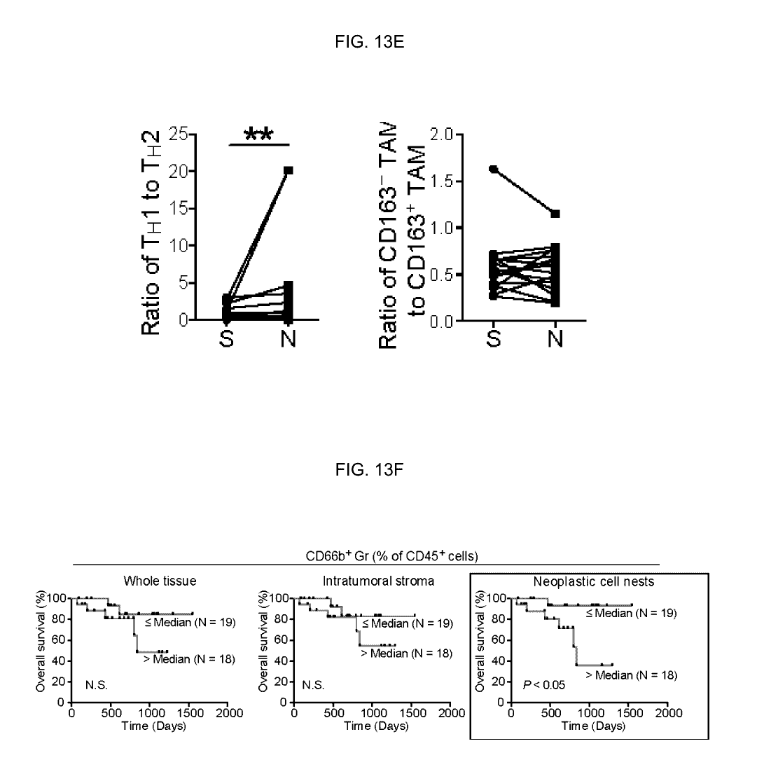

Differential distribution patterns of immune infiltrates in tumor regions. Coukos and colleagues previously revealed positive correlations of tumor-infiltrating lymphocytes within intratumoral regions of ovarian cancers as correlating with clinical outcome (Zhang et al, N Engl J Med 348, 203-213 (2003); incorporated by reference herein), and Galon and colleagues have developed Immunoscore to characterize immune infiltrates across a spectrum of solid tumors (Galon 2006 supra and Fridman 2012 supra). However, spatial characteristics of lymphoid versus myeloid lineages, and the phenotype of those lineages, e.g., pro-tumoral versus anti-tumoral (Palucka and Coussens 2016 supra), has not previously been possible with conventional IHC- or IF-based approaches. To understand immune complexity and phenotype based on tissue context, tissue-contextual information was added to image cytometry analytics by adapting a mathematical morphology-based tissue segmentation strategy (Serra, Image analysis and mathematical morphology (Academic Press, 1983) and Huang & Wang, Pattern Recognition 28, 41-51 (1995); both of which are incorporated by reference herein). Neoplastic cell-biomarker IHC following lymphoid and myeloid panels was performed. In these assays, p16 expression was used to indicate HPV-positivity (N=21) versus EpCAM for HPV (p16)-negative tumors (N=16), and one case (Core #37) where negative staining for both markers excluded the sample for analysis. Digitized structural elements of positively stained areas were computationally processed by thresholding methods, and each tissue core classified into neoplastic cell nest versus intratumoral stromal regions excluding blank regions without tissue (See FIG. 5A). Results from tissue segmentation were validated by quantification of neoplastic cells by image cytometry, and confirmed that the vast majority of neoplastic cells categorized to neoplastic cell nest regions (FIG. 5A). Leukocyte composition analysis based on tissue segmentation dissecting the whole tissue-based result into neoplastic nests versus intratumoral stromal regions revealed significantly higher percentages of TH2, TH0 and B cell in stroma, while TH1 tended towards higher percentages in neoplastic cell regions (FIG. 5B). Differential distribution patterns of TH1 to neoplastic cell nests and TH2 to intratumoral stroma in HNSCC was observed (FIGS. 5C and 13A). These observations were further supported by statistically high ratios of TH1/TH2 in neoplastic cell regions (FIG. 5D). These analyses were further divided based on HPV-status, wherein that both HPV-positive and negative subgroups showed a tendency towards intratumoral TH1 polarization (FIGS. 13B, 13C, 13D, and 13E). In addition, HPV-positive tumors showed high CD163−/CD163+ ratio of TAMs, associating with TH1-polarized phenotype of macrophages (FIG. 13D).

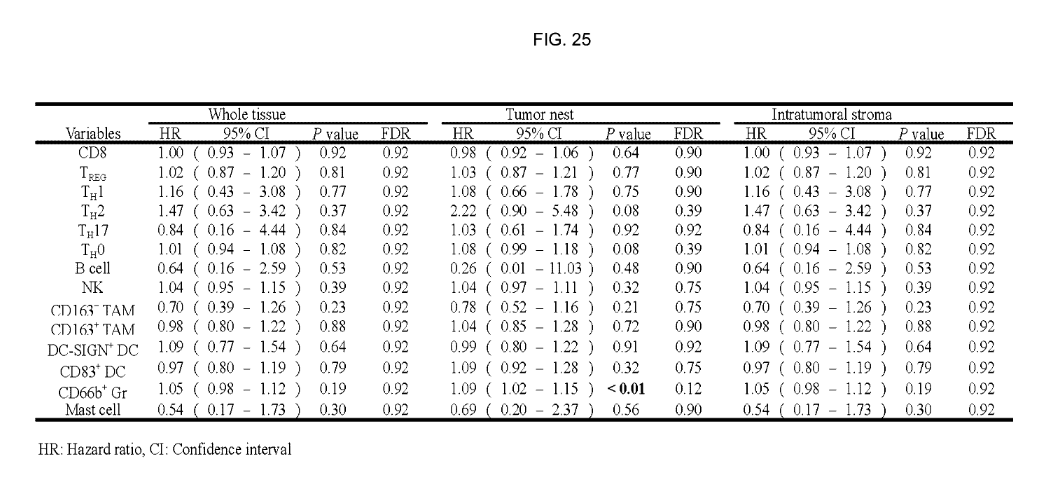

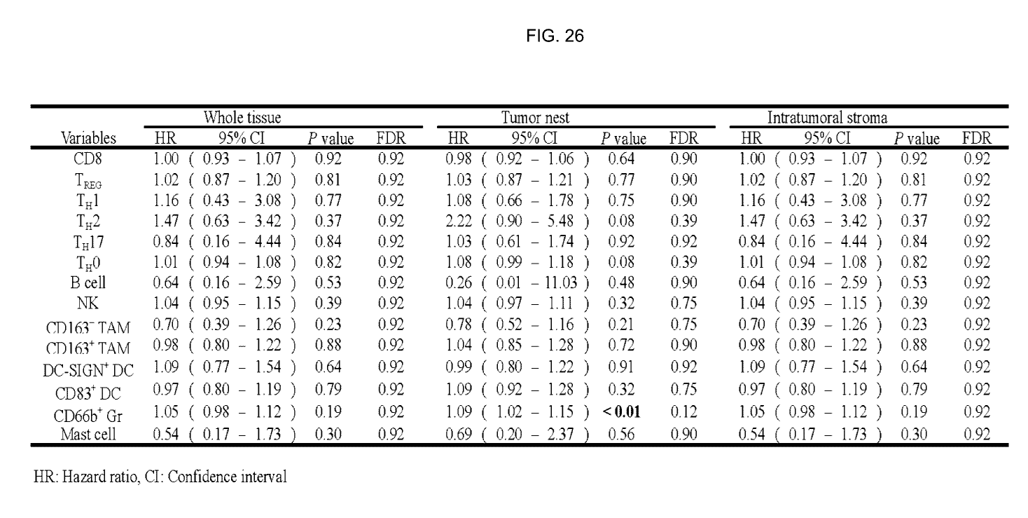

Prognostic association of differential immune profiles in the tissue context was then performed. Using Cox regression analyses, CD66b+ Gr in neoplastic cell regions was found to be associated with a high hazard risk, although whole tissue and stroma-based data did not show statistical significance (FIG. 25). This result was further confirmed by adjustment for HPV-status, showing consistent tendencies of negative correlation between prognosis and CD66b+ Gr in neoplastic cell regions (FIG. 26). Kaplan-Meier analysis revealed that high infiltration of CD66b+ Gr in neoplastic nests correlated with shorter overall survival although whole tissue or stroma-based data did not show statistical differences likely owing to small sample size (FIGS. 5E and 13F). Notably, intratumoral TH1 orientation analyzed by TH1/TH2 ratios showed inverse correlations with frequency of intra-tumor nests of CD66b+ Gr in neoplastic versus stromal areas (FIG. 5E). Together, the imaging approach revealed distinct immune-related profiles in stroma versus neoplastic cell areas, indicting presence of tissue context-associated regional tumor characteristics, correlating with prognosis and potential outcome (FIG. 24).

### Example 6

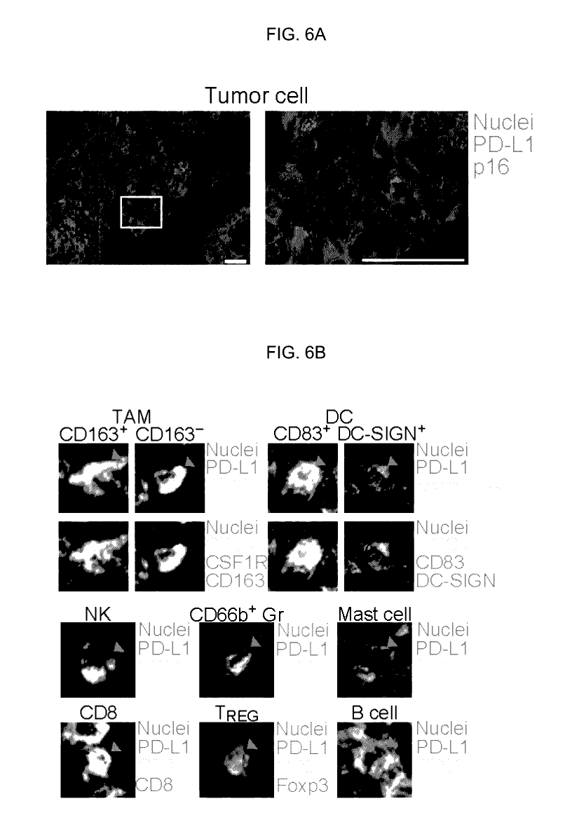

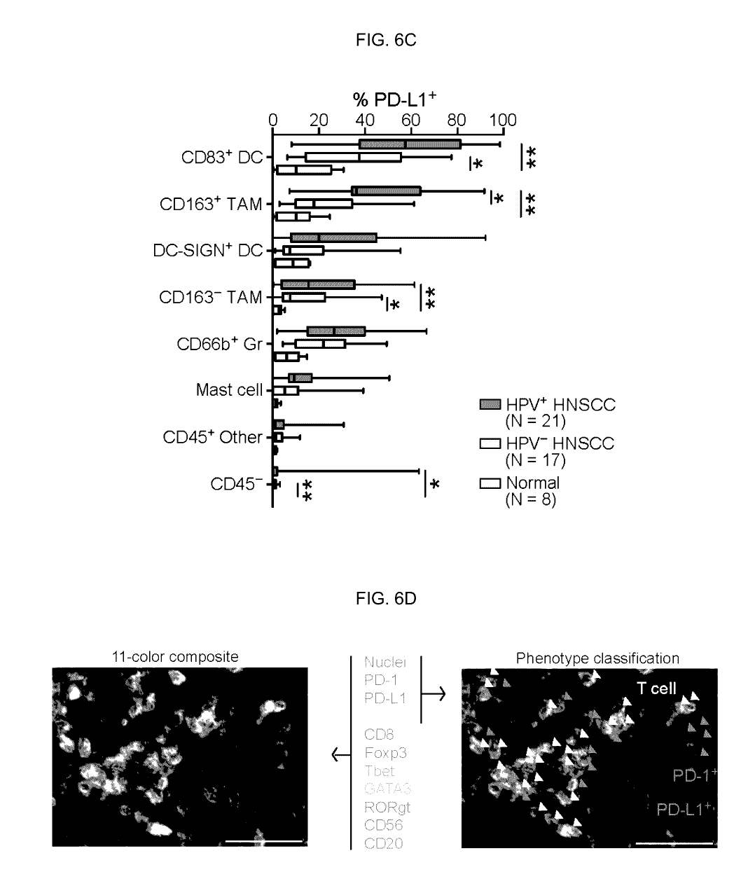

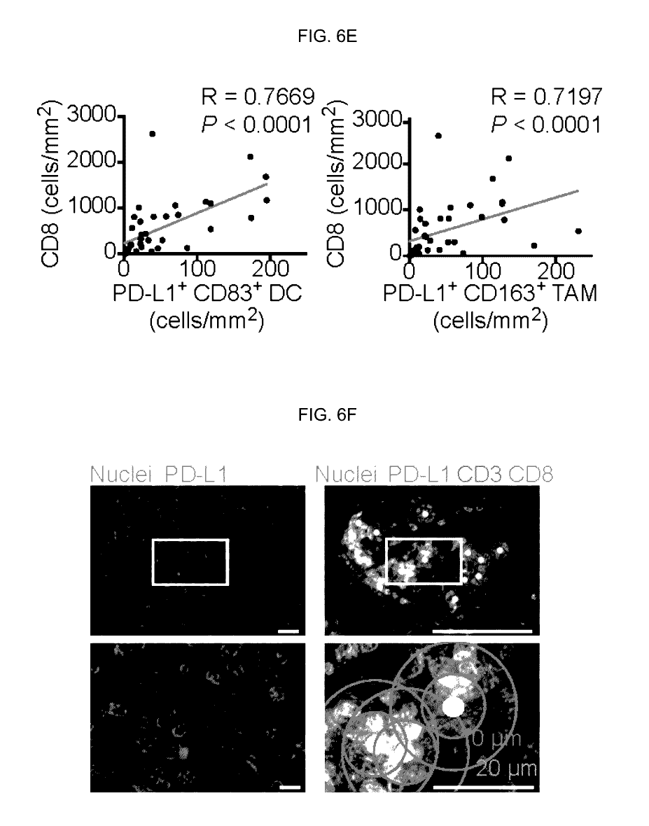

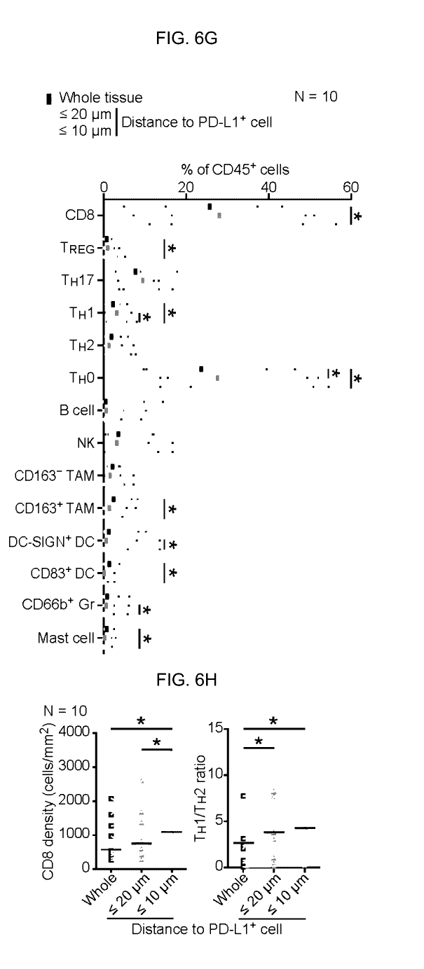

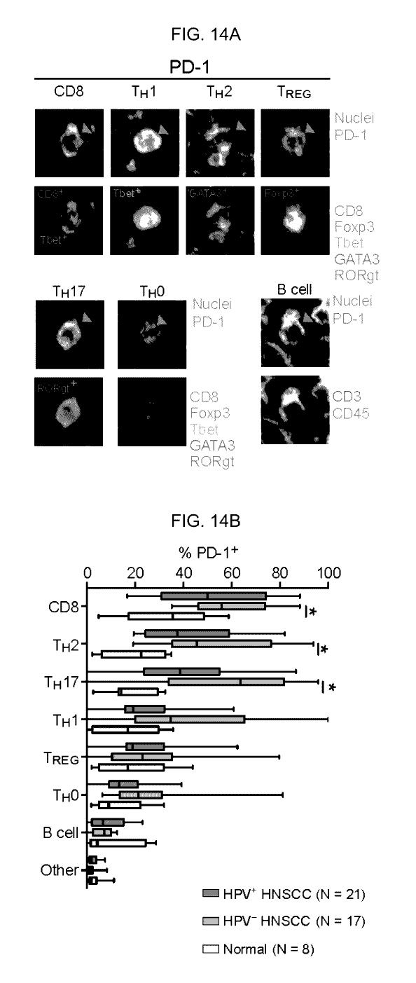

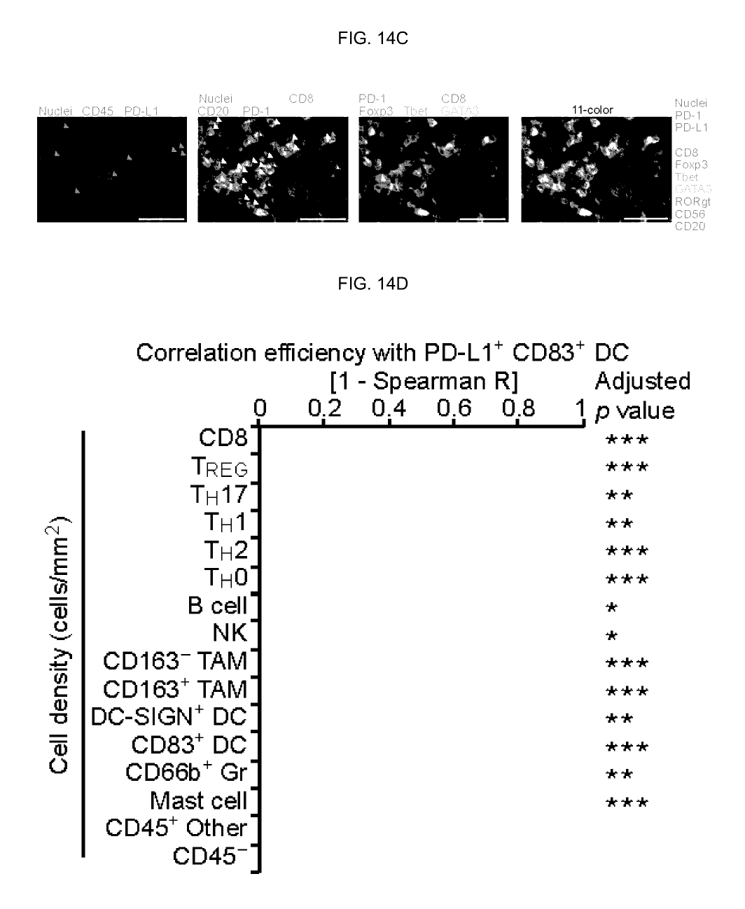

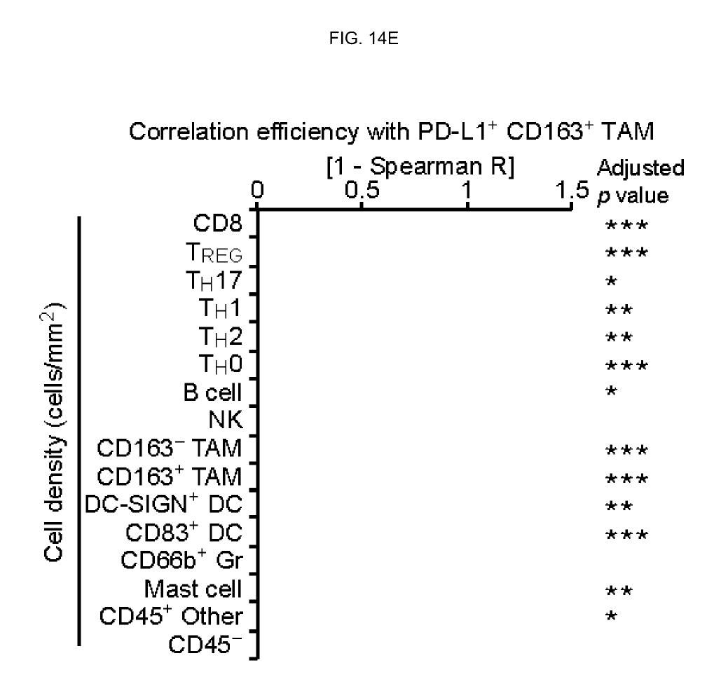

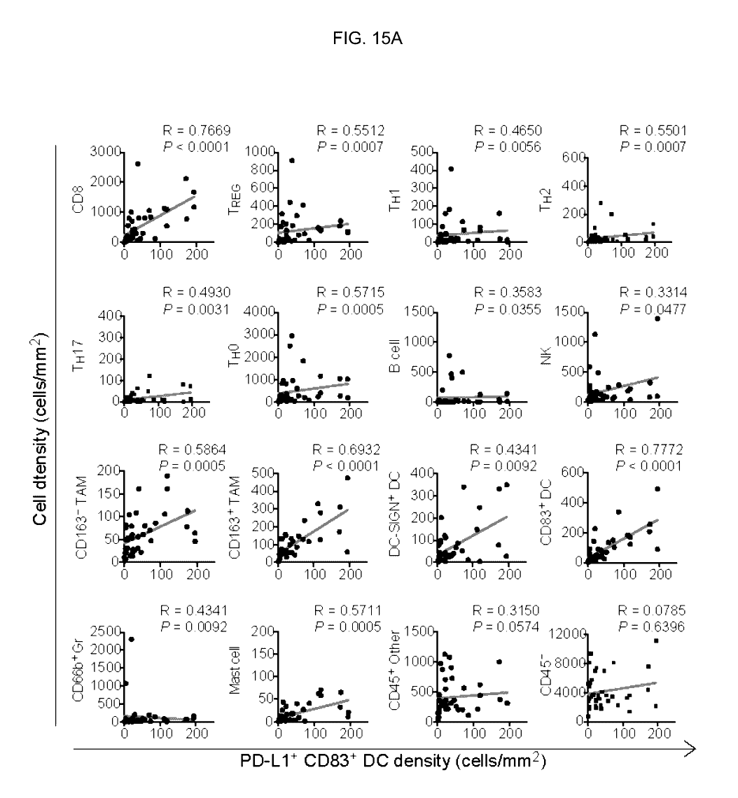

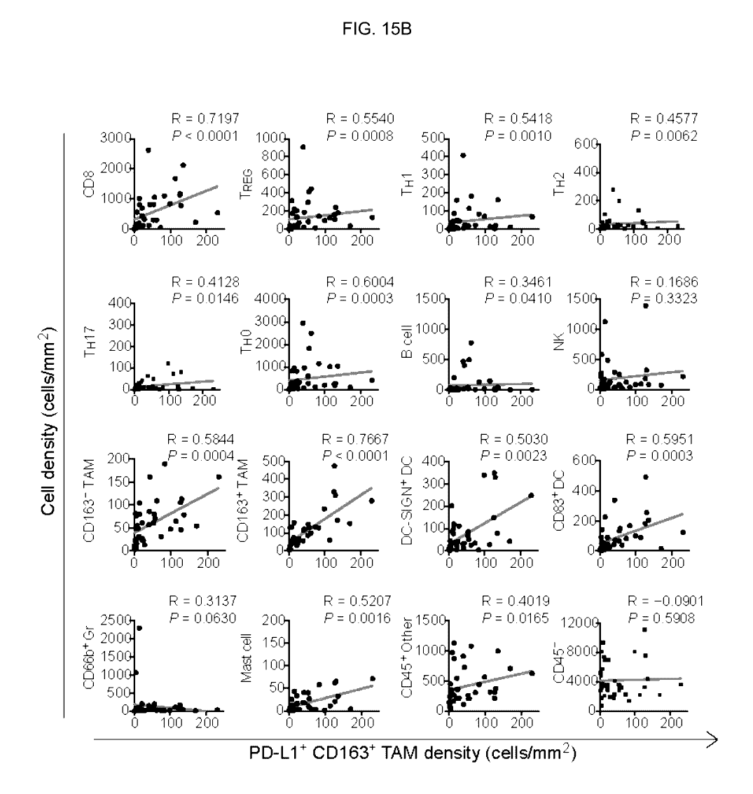

Regional polarization of immune complexity is associated with immune checkpoint expression in neoplastic and immune cells. Since the disclosed imaging methods allow visualizing and quantifying colocalization of protein biomarkers, the ability of the methods to perform in situ detection of select immune checkpoint molecules expressed on various cell populations identified with the multiple lineage markers shown in FIG. 23 was explored. In particular, expression of programmed cell death 1 (PD-1) and its ligand (PD-L1), which together regulate T cell activation and tolerance, and also reflect therapeutic targets in a wide range of cancer types including HNSCC was assessed (Pardoll D M, Nat Rev Cancer 12, 252-264 (2012); Topalian et al, N Engl J Med 366, 2443-2454 (2012); and Lyford-Pike et al, Cancer Res 73, 1733-1741 (2013); incorporated by reference herein). PD-L1 was included in both the lymphoid and myeloid biomarker panels FIGS. 22A and 22B, and PD-L1 expression was analyzed in the TMA of HNSCC. IHC analyses revealed PD-L1 expression on neoplastic cells (FIG. 6A), while PD-L1 expression was also observed in various immune cell lineages including CD163+ and CD163-TAMs, CD83+ and DC-SIGN+ DCs, NK, CD66b+ Gr, mast cells, T cells, and B cells (FIG. 6B), in support of previous reports (Nishimura & Honjo, Trends Immunol 22, 265-268 (2001); Iwai et al, Proc Natl Acad Sci USA 99, 12293-12297 (2002); Ishida et al, Immunol Lett 84, 57-62 (2002); de Kleijn et al, PLoS One 8, e72249 (2013); Nakae et al, J Immunol 176, 2238-2248 (2006); Latchman et al, Proc Natl Acad Sci USA 101, 10691-10696 (2004); Khan et al, Nature Comm 6, 5997 (2015); all of which are incorporated by reference herein) Simultaneously, PD-1 expression was analyzed with the lymphoid biomarker panel, and PD-1 expression was observed on CD8+ T Cells, CD8− T cells, and partially on B cells (FIG. 14A). To quantitatively verify these observations, positive percentages of PD-1/PD-L1 expression in each cell lineage were then quantified by image cytometry, and transversely analyzed across cell lineages together with subclassification of tumor/normal tissue types. Among all cell lineages analyzed in the cohort, the highest frequency of PD-L1 expression was observed on myeloid cells rather than CD45 negative cells including neoplastic cells (FIG. 6C). In comparison with normal pharynx, HNSCC tissues showed significantly high PD-L1 expression on myeloid cells and PD-1 on CD8, TH2, and TH17 particularly in HPV-associated tumors (FIGS. 6C and 14B), indicating presence of active PD-1/PD-L1 signaling in the TME of HNSCC.

Based on these observations that PD-L1+ cells were frequently spatially surrounded by CD3+ T cells in HNSCC (FIGS. 7D and 14C), a hypothesis that there was a relationship between PD-L1+ cells and surrounding cell lineages in terms of density and spatial distributions was developed. To test this hypothesis, whole tissue-based correlation analysis of cell densities assessing 16 cell lineages and PD-L1+ cells (FIGS. 14D, 14E, 14F, and 14G) was performed. Strong correlations were observed between CD8 density and PD-L1-expressing CD83+ DCs and CD163+ TAMs, which appeared as the highest PD-L1 expressing cells among all cell lineages (FIGS. 6C and 6E). To further focus on localization of immune cell lineages and PD-L1+ cells, the distribution of cell lineages localized to surrounding areas of PD-L1+ cells in the PD-L1 highest cores in the TMA (N=10) was evaluated. PD-L1− neighbor cells within a distance of 10-20 μm to PD-L1+ cells were quantified (FIG. 6F). In comparison to the whole tissue-based data, cell composition of PD-L1-neighbor cells within 10 μm showed a robust increase of CD8 (FIG. 6G). Moreover, CD8 density significantly increased with more proximal distance from PD-L1+ cells (FIG. 6H), indicating a spatial distribution-based association between CD8 and PD-L1 upregulation. Interestingly, TH1 and TH2 analysis revealed an opposite tendency in terms of frequency of PD-L1 surroundings (FIG. 6G), with TH1/TH2 ratio significantly elevated in the 10 and 20 μm-range to PD-L1+ cells, showing a potential association between PD-L1 expression and TH1-based regional polarization in immune complexity phenotypes. These results demonstrate the capability of multiplex IHC-based image cytometry analysis, enabling phenotyping, density, composition, and spatial distribution of various cell lineages with regards to heterogeneous TMEs.

### Example 7

Methods. Clinical samples and TMA construction: Human FFPE samples of HNSCC were obtained from the Oregon Health and Science University (OHSU) Knight Cancer Institute Biolibrary, and the OHSU Department of Dermatology research repository. A total of 38 oropharyngeal squamous cell carcinoma specimens were used to create a tissue microarray (TMA) for analysis. All tumor samples were reviewed by a head and neck pathologist to select representative tissue with dense, non-necrotic tumor. As a control, a total of 10 adult palatine and lingual tonsillectomy specimens removed for benign, non-inflammatory indications (i.e. obstructive sleep apnea) were included in the TMA. The TMA was created using an automated microarrayer (3D Histech TMA Master; Budapest, Hungary), which took 2 mm cores from the selected area of the donor block and placed them into an array on the recipient block. All tumors were staged according to the 7th edition AJCC/UIC TNM classification and cohort characteristics shown in FIG. 24. HPV-status was determined by p16 staining and/or by quantitative PCR when available. Two cases of benign tonsillectomy specimen were excluded due to insufficient amount of tissue in the TMA.

Sequential IHC: Sections (5 μm) of FFPE tissues were placed in a 60° C. heat chamber for 30 min, deparaffinized with xylene, and rehydrated in serially graded alcohols to distilled water. Slides were stained by hematoxylin (S3301, Dako) for 1.0 min, mounted with TBST buffer (0.1 M TRIS-HCl, pH 7.5, 0.15 M NaCl plus 0.05% Tween-20), and coverslipped with Signature Series Cover Glass (12460S, Thermo Scientific), followed by whole tissue scanning using an Aperio ImageScope AT (Leica Biosystems) at 20× magnification. After decoverslipping of slides with 1.0 min-agitation in TBST, peroxidase activity was blocked by 0.6% hydrogen peroxidase in methanol for 30 min, and slides were subjected to heat-mediated antigen retrieval immersed in citrate buffer (10 mM citric acid, 0.05% Tween® 20, pH 6.0) for 15 min. Then, sequential IHC including iterative cycles of staining, scanning, and antibody/chromogen stripping was modified from Stack 2014 supra, Glass 2009 supra and Tramu 1978 supra. After a protein blocking step with 5.0% goat serum, 2.5% BSA, and 0.1% Tween-20, unlabeled primary antibodies were added to sections at the indicated dilution shown in FIGS. 22A and 22B. After washing in TBST buffer, slides were visualized with either an anti-mouse or anti-rabbit Histofine Simple Stain MAX PO horseradish peroxidase-conjugated polymer (Nichirei Biosciences Inc.), followed by peroxidase detection with AEC for the indicated incubation time specified in FIGS. 22A and 22B. Coverslipping, whole tissue scanning, and decoverslipping were performed as described above. Following chromogenic destaining in an alcohol gradient, antibodies were stripped by a microwave heat treatment for 90 sec at maximum power and 15 min at 10% power in Antigen Retrieval Citra Solution (BioGenex) resulting in a temperature of at least 90° C. for a typical volume used. Slides were then restained sequentially in the indicated order shown in FIGS. 22A and 22B. Two forms of negative controls were used during these analyses: Slides for conventional negative controls were treated with 2.5% goat serum in PBS without primary antibodies; Slides for sequential IHC negative controls were used for confirmation of complete antibody/signal stripping (FIG. 8A).

Image coregistration: Coregistration of serially scanned images was performed by an in-house pipeline, “Alignment_Batch.cppipe” using CellProfiler Version 2.1.1 (Carpenter 2006 supra): delta-X and Y location among serially scanned images were computed based on manually selected single structures such as cells, vessels, and edges of tissues. Then, images were exported as non-compressed TIFF images using ImageScope Version 12.1.0.5029 (Aperio Technologies Inc.) based on alignment information. Pseudocodes for algorithms used are shown below in Examples 8 and 9.

Single cell-based quantification in image cytometry: Single cell-based segmentation and quantification of staining intensity was performed using a novel automated image segmentation pipeline “CellID_FlowCyt—6.9.15” using CellProfiler Version 2.1.1. This customized pipeline used several AEC-stained images for protein level quantification, and one hematoxylin-stained image for cell segmentation. First, individual RGB channels were extracted from the hematoxylin-stained image. Next, pixel intensities for images were inverted to optimize the algorithm's ability to detect cells. Cell segmentation of the hematoxylin-stained image was then performed using a built-in watershed segmentation algorithm as described in Wahlby 2010 supra. Prior to segmentation, a built-in thresholding method was utilized to identify local intensity maxima and minima, as well as to differentiate foreground from background pixels, as described previously in Padmanabhan 2010 supra. The nature of multiplex staining allows segmentation results (referred to as “objects”) to be used as templates for staining quantification of serially scanned AEC images. The color channel specific to AEC staining was extracted from each AEC-stained image. Using objects from the watershed segmentation, cell coordinates were overlaid onto these AEC channels, thus locating each cell on the protein-stained images. Subsequently, measurements of pixel intensity were extracted and recorded. CellProfiler also measured 26 different area and shape features of cells in the image. A color map was then constructed of all identified cells by assigning a number to each pixel within each individual cell. Color maps were then saved for image cytometry analysis. Finally, all pixel intensity and shape-size measurements were saved to a file format compatible with flow and image cytometry data analysis software, FCS Express 5 Image Cytometry Version 5.01.0029 (De Novo Software). Pseudocodes for algorithms used are shown below in Examples 8 and 9.

Image visualization: Coregistered images were converted to pseudo-colored single-marker images in ImageJ Version 1.48. Following coregistration, exported images were processed using an ImageJ plugin, Color Deconvolution for AEC and hematoxylin signal separation. Following pixel histogram optimization, images were then inverted and converted to gray-scale, followed by pseudo-coloring in ImageJ.

Computational algorithms for tissue segmentation: Tissue segmentation of neoplastic cell nests and intratumoral stromal regions was performed using an in-house application, “Tissue Segmentation Version 1.3” based on tumor marker-IHC images (p16 for HPV-positive, and EpCAM for HPV-negative HNSCC). Firstly, the region with tissues, defining the region of interest (ROI), and the blank region without tissues were classified based on a calculation of a maximum thresholding for each image, followed by an image-cleaning algorithm with mathematical morphology operations including opening and closing, and a fill holes operation Serra J, Image Analysis and Mathematical Morphology (Academic Press Inc., 1983); incorporated by reference herein) Secondly, the tumor nest region was calculated by an automated thresholding with the Huang fuzzy method performed only on the ROI, followed by the image-cleaning algorithm as described above. Stromal regions were calculated by a subtraction of tumor nest regions from the ROI. Finally, a corresponding hematoxylin image was cropped fitting to the result of tissue segmentation such as neoplastic cell nests and/or intratumoral stroma, and analyzed with serially scanned AEC images by the pipeline “CellID_FlowCyt—6.9.15” using CellProfiler Version 2.1.1.

Statistics Kruskal-Wallis and Wilcoxon signed rank tests were used to determine statistically significant differences in un-paired and paired data. Spearman correlation coefficient was used to assess correlations of cell percentages and densities among cell lineages. Overall survival was estimated using Kaplan-Meier methods, and differences were assessed with log-rank tests. Cox proportional hazards regression was used to assess the relationship of overall survival with cell densities and percentages, unadjusted and adjusted for HPV-status. An unsupervised hierarchical clustering was performed with Ward's minimum variance method (“hclust” from “R”). P values were adjusted for multiple comparisons using Benjamini-Hochberg false discovery rate (FDR) adjustments. All statistical calculations were performed by R software, version 3.2.3 and SAS software version 9.4. P<0.05 was considered statistically significant.

### Example 8

Psuedocode used in image coregistration. # The inputs are the script, and an array of the image files (in most cases 12 total). It is assumed the first image is H&E, and is only used for alignment and segmentation.

```
script, image1, image2, image3, image4, image5, image6, image7, image8, image9,
image10, image11, image12 = inputs
″″″ Alignment ″″″
# The implemented alignment algorithm aligns all images to the first image in the list.
# Displays each image, in which the user must select a common point
def find_offsets(all_image_files):
  file1 = all_image_files[0]
  # Reference coordinates to be compared to each other image's coordinates
  file1_coordinates = [ ]
  # X and y offsets. Image 0 (file1) has an offset of 0,0 because it's the reference image.
All other images are aligned to it.
  offsets = [ (0,0) ]
  # When the user right clicks in the graph, it adds the coordinates to the coordinate list.
When the window is closed, the script continues
  # IMPORTANT: Coordinate lists must be same length.
  def onclick(event, coordinate_list):
    coordinate_list.append( (event.x, event.y) )
  figure1.showimage(file1)
  figure1.connect(″right_click_event″, onclick(file1_coordinates))
  figure1.show( )
  # Iterate through all images (besides first ″reference″ image)
  for x in range(1, len(all_image_files)):
    filex_coordinates = [ ]
    while len(filex_coordinates) != len(file1_coordinates):
      figure1.showimage(all_image_files[x])
      figure1.connect(″right_click_event″, onclick(filex_coordinates))
      figure1.show( )
    # Offset compared to reference image (file1). Find difference between reference
image and current image points.
    x_offset = int(sum(filex_coordinates[a][0] - file1_coordinates[a][0] for a in range(0,
len(filex_coordinates)))/len(filex_coordinates))
    y_offset = int(sum(filex_coordinates[a][1] - file1_coordinates[a][0] for a in range(0,
len(filex_coordinates)))/len(filex_coordinates))
    # Add this image's coordinates to offsets array
    offsets.append( (x_offset, y_offset) )
return offsets
```

### Example 9

Pseudocode for cell segmentation and signal quantification. # The inputs are the script, and an array of the image files (in most cases 12 total). It is assumed the first image is H&E, and is only used for alignment and segmentation

```
script, image1, image2, image3, image4, image5, image6, image7, image8, image9,
image10, image11, image12 = inputs
″″″ Segmentation and Data Collection ″″″
# This code assumes the images have been aligned
max_cell_size = 100
min_cell_size = 10
# Only segments one image, and returns labels to be applied to all images after alignment
def segment(image):
  # Watershed segmentation based on a distance or gradient transform
  img = image.rgb2gray( )
  # distance = distance_transform(img)
  gradient = gradient(img)
  local_maxi = find_local_maxima(gradient)
  markers = label(local_maxi)
  labels = watershed(-gradient, markers, mask=image)
  # Remove cells that are too big or too small
  temp_props = regionprops(labels, image)
  for cell in temp_props:
    if cell[′area′] < min_cell_size or cell[′area′] > max_cell_size:
      labels[labels == cell[′label′]] = 0
  return labels
# Collect stain data from all images except first (assumes first is H&E)
def collect_data(all_image_files, labels):
  # Make dataframe tostore protein intensities
  save_data = pd.DataFrame( col1=labels[′label′], col1_name=′Cells′ )
  # Record label numbers, in order
  save_data[′Labels′] = (x[′label′] for x in regionprops(labels))
  # In each image, measure the AEC level of each label
  for x in range(1, len(all_image_files)):
    add_data = [ ]
    for cell in regionprops(labels):
      # Access pixels of each label, overlay the image onto pixels, and record
    normalized RGB intensities
      # These RGB values are normalized based on AEC staining (3-amino-9-
    ethylcarbazole). Values vary based on staining method
      add_data.append(a[′pixels′]read_pixels( mask=all_image_files[x],
    normalize=(0.274, 0.679, 0.680) ))
      save_data[′Image_′ + x + ′_AEC′] = add_data
  # Save data is organized as a pandas dataframe of rows and columns.
    # The first column is the label count for individual (1 to [number of labels])
    # The 2nd-12th columns are the AEC intensity values of each label (cell) on each
  image.
  save_data.io.save(″Image_Cytometry_Results″)
# Make arrays of all input images
all_images = [image1.asarray, image2.asarray, image3.asarray, image4.asarray,
image5.asarray, image6.asarray, image7.asarray, image8.asarray, image9.asarray,
image10.asarray, image11.asarray, image12.asarray]
# Dictionary of image offsets
image_offsets = [ ]
# Find offsets
image_offsets = find_offsets(all_images)
# Align images based on offsets
for x in range(0, len(image_offsets))
  all_images[x] = realign_pixels( all_images[x], image_offsets[x] )
# Now that images are aligned, segment cells in H&E image and collect data
collect_data( all_images, segment(all_images[0]) )
```

### Example 10

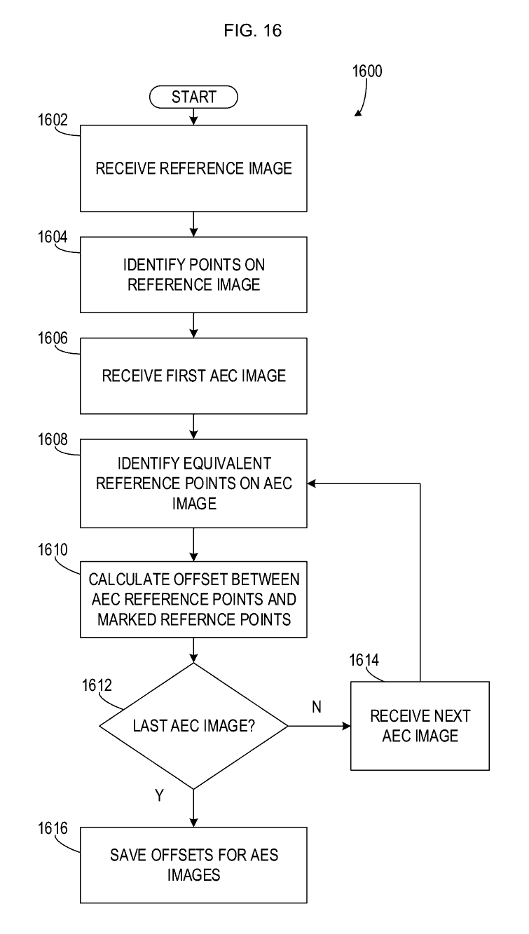

Image co-registration method. FIG. 16 shows an example of a method 1600 to coregister a set of images of AEC-stained samples to a reference image. In embodiments, the reference image received at 1602 can include a digital image of a section treated with a stain that allows visualization of cellular structures (i.e., a structure-stained image); for example, a digital image of a hematoxylin-stained sample. At 1604, a number of reference points are identified on the reference image. For example, these reference points may be fiducial markers embedded within the sample, fiducial markers placed on the slide to which the sample is affixed, or points that coincide with specific features of the sample such as cell or vessel structures. Those skilled in the art will understand that these reference points may be identified automatically by an image processing algorithm or selected manually depending on the application. Beginning at 1606, the method receives the first of a set of AEC-stained images, wherein the AEC-stained images are captured from the same sample as the reference images. At 1608, a set of equivalent reference points are identified on the AEC-stained image, the equivalent reference points having spatial and/or structural correspondence to the reference points identified in the reference image. At 1610, an offset is calculated between the reference points of the reference image and the AEC-sample's equivalent reference points. The calculated offset is a transformation that brings the two sets of points into alignment so that the reference image and AEC image are aligned or co-registered. In particular embodiments, this transformation may include a simple translation of X and Y coordinates (i.e., delta-X and delta-Y offsets) for pairs of images that are not rotated or magnified relative to one another. In other embodiments, the offset may be a more general transformation which allows for translation, rotation, and scaling such as an affine transform. At 1612 and 1614, the remaining AEC images are looped over, calculating for each AEC image an offset that effects registration of that AEC image to the original reference image. At 1616, the set of offsets produced by method 1600 are saved so that they may later be applied to coregister all AEC images to the reference image.

### Example 11

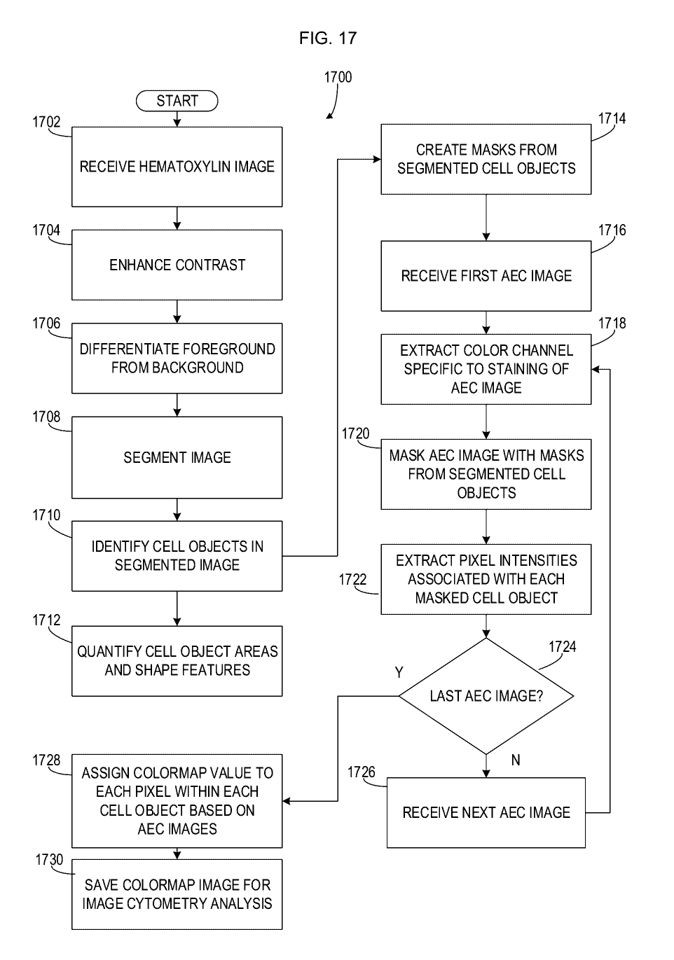

Cell segmentation and quantification method. FIG. 17 shows an example of a method 1700 for cell segmentation and quantification in accordance with the systems and methods described herein. This method 1700 may be used, for example, to gather data to perform image cytometry analysis as described in this disclosure. At 1702 a hematoxylin image is received to serve as a stained-structure image for cell segmentation. In embodiments, the hematoxylin image can be converted to grayscale format if it is received in RGB format. The hematoxylin image is enhanced at 1704 to increase the contrast of the cellular structures compared to background of the image. In particular embodiments, this enhancement may be performed by inverting the pixel intensities or otherwise scaling the image lookup table to improve detection of cells in the image. Operations are performed at 1706 to differentiate the foreground from background. These operations may include, for example, thresholding to identify local intensity maxima and minima as part of the differentiation procedure. At 1708, a segmentation algorithm is applied to locate cell boundaries within the image. In particular embodiments disclosed herein, segmentation is performed using a watershed segmentation algorithm. Those having skill in the art will recognize that alternative segmentation algorithms may be employed, including level set methods, fast marching methods, thresholding methods, adaptive thresholding methods, edge-based methods, histogram-based methods, clustering methods, region-growing methods, variational methods, multi-scale methods, model-based methods, or other segmentation approaches known in the art. At 1710, cell objects are identified in the segmented image; these cell objects are used as masks in subsequent processing steps. Morphological aspects of these identified cell objects may be characterized at 1712 to quantify, for example, cell areas, cell shape descriptors, or other features. At 1714 the cell objects identified at 1710 are used to create a set of masks to be used to interrogate cell contents of a set of AEC images. Upon receipt of a first AEC image at 1716, the color channel specific to the AEC stain is extracted 1718, overlayed at 1720 with the masks created from the segmented cell objects from 1714, and pixel intensity measurements extracted for each cell associated with a mask at 1722. This process is repeated in a loop-wise manner at 1724 and 1726 until all AEC images have been interrogated. At 1728, a color map is generated, where in a color map value is assigned to each pixel within each cell object based on the analysis of the set of AEC images. At 1730, this color map and associated data is saved in an appropriate format for later image cytometry analysis.

### Example 12

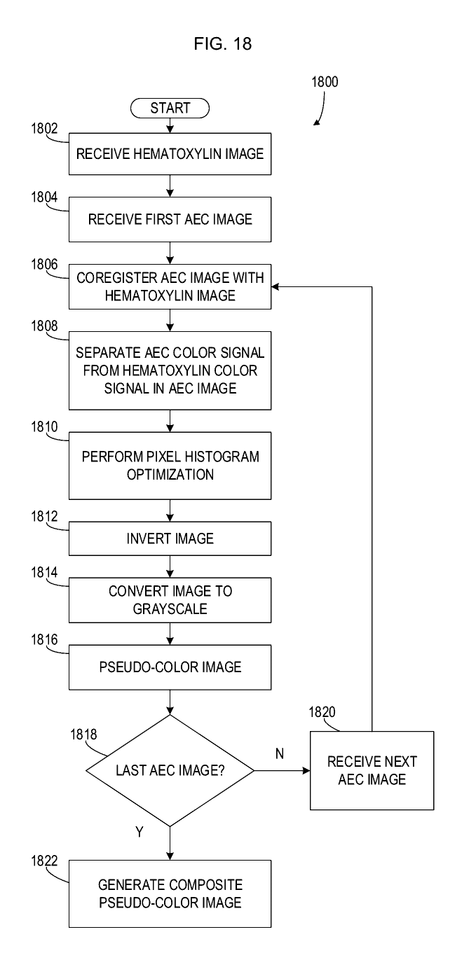

Composite pseudo-color image visualization method. FIG. 18 example of a method 1800 for visualizing extracted AEC data as a composite pseudo-color image in accordance with the systems and methods described herein. A hematoxylin image (i.e., the structure-stained image) and AEC image are received at 1802 and 1804, respectively, and coregistered at 1806. At 1808, the AEC image is processed to remove or separate the contribution of hematoxylin color signal within the image. This separation can be performed, for example, using a color deconvolution approach as known in the art. Pixel histogram optimization is performed at 1810, then inversion of the image at 1812, followed by conversion if the image to grayscale at 1814. The resultant image is assigned a pseudo-color at 1816 to serve as an identifier of the specific marker associated with the specific AEC staining. At 1818 and 1820, a looping structure is engaged to process additional AEC images in the same manner and assign unique pseudo-colors corresponding to the specific markers captured in each of the AEC images. Once all AEC images are processed according to steps 1806 through 1816, a final composite pseudo-color image is generated at 1822.

### Example 13

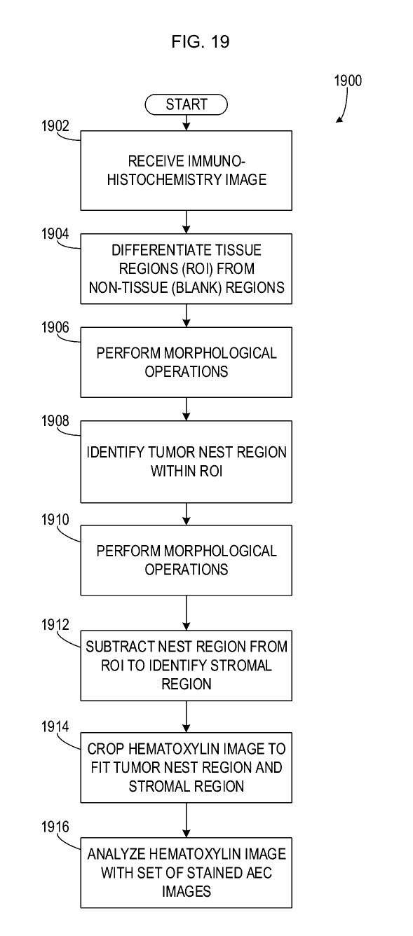

Tissue segmentation and quantification method. FIG. 19 shows an example of a method 1900 for tissue segmentation and quantification in accordance with the systems and methods described herein. Upon receipt of an IHC image at 1902, the tissue regions of the IHC image (i.e., the region of interest (ROI)) are differentiated from the blank, non-tissue-containing regions at 1904. In particular embodiments, the tissue and non-tissue regions can be classified using an appropriate segmentation or thresholding technique, for example by a maximum thresholding approach. The ROI is further processed at 1906 using morphological operations to clean the ROI. Examples of morphological operations include erosion and dilation, opening and closing, filling, and filtering of pixels cluster having prescribed areal or shape properties. At 1908, the tumor nest region within the ROI is identified. This identification can be performed using an appropriate segmentation or thresholding technique. For example, in the examples described herein, the Huang fuzzy method is used to identify the tumor nest region. At 1910, morphological operations as described above are performed to clean the tumor nest region, and then at 1912 the cleaned tumor nest region is subtracted from the ROI to identify the stromal region. At 1914 a corresponding hematoxylin image is cropped and fit to the segmented tumor nest and/or stroma regions, and at 1916, the hematoxylin image is used in conjunction with a set of stained AEC images. In particular embodiments, the analysis at 1916 can include application of the method 1700 described previously for image cytometry.

### Example 14

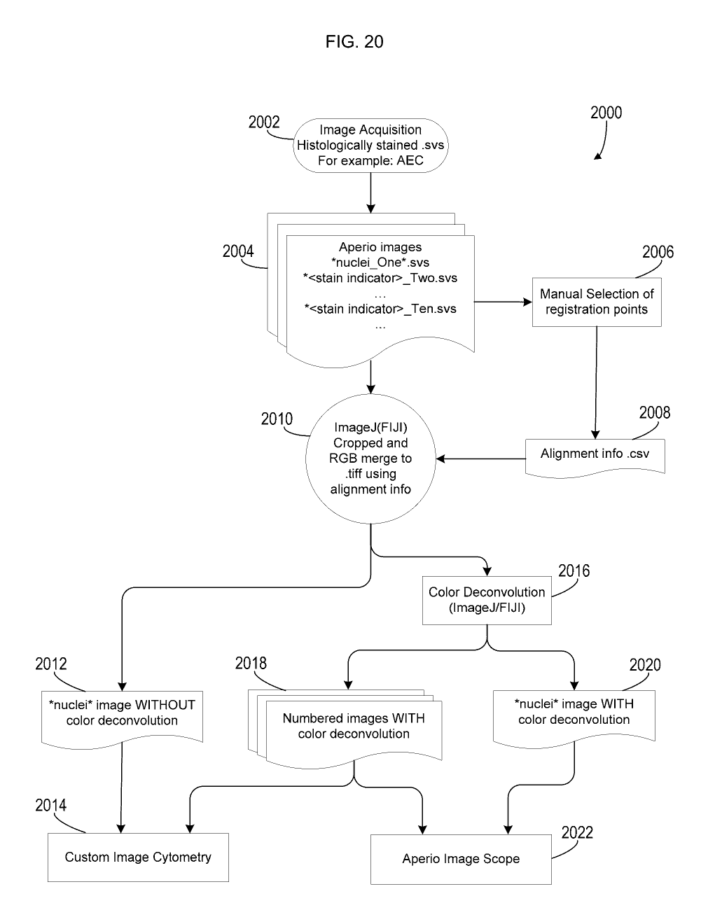

Sample Workflow 1. FIG. 20 shows an example of a workflow 2000 for processing images and quantifying results using the methods disclosed herein. At 2002, digital images of a structure-stained and serially labelled (e.g., AEC) sample are acquired and assigned filenames at 2004 to facilitate processing. At 2006, a set of reference points are selected manually for the registration of the set of images, and at 2008 alignment information is saved to a file for later access. At 2010, the image processing program ImageJ/FIJI is used to align and crop the set of images, and merge the RGB channels. The structure-stained RBG-merged image at 2012 is passed to a custom program at 2014 for image cytometry analysis (for example, calculation of cell areas and shape factors). All RBG-merged images are also passed to a color deconvolution algorithm at 2016, with the deconvolved labelled images saved at 2018 and the deconvolved structure-stained image saved at 2020. At 2020, all deconvolved images are passed the Aperio Image Scope image processing program for further analysis and review.

### Example 15

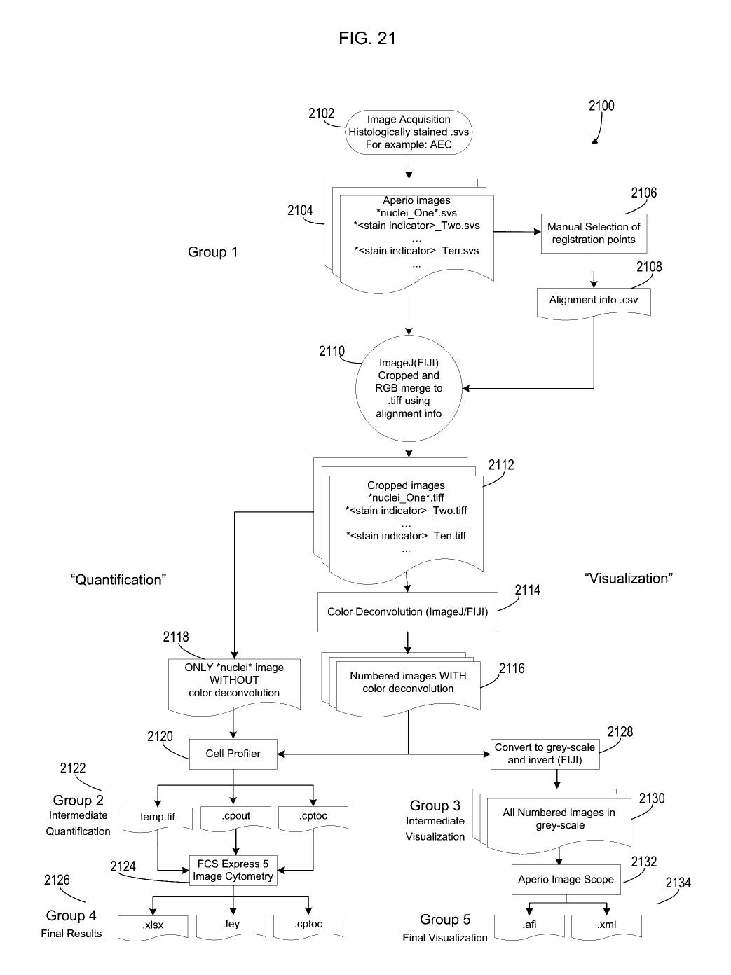

Sample Workflow 2. FIG. 21 shows an example of a workflow 2100 for processing images and quantifying results using the methods disclosed herein. At 2102, digital images of a structure-stained and serially labelled (e.g., AEC) sample are acquired and assigned filenames at 2104 to using a standardized naming convention to facilitate processing. At 2106, a set of reference points are selected manually for the registration of the set of images, and at 2108 alignment information is saved to a file for later access. At 2110, the image processing program Image J (FIJI) is used to align and crop the set of images, and merge the RGB channels. At 2112, the RGB-merged images are assigned new filenames according to a standardized naming convention. At 2114 color deconvolution is applied to the set of RGB-merged images as part of the cell segmentation and analysis procedure, and saved again at 2116. At 2118, the structure-stained image which has not undergone color deconvolution is passed to an image processing program (CellProfiler) at 2120 for quantification and a set of output files are generated at 2122. These output files are used as input to an image cytometry program (FCS Express 5) at 2124 and a final set of quantification output files are saved at 2126.

Returning to the deconvolved images at 2116, this set of files is passed to an image processing program at 2128 (ImageJ/FIJI), where they are converted to grayscale and inverted as part of a visualization pipeline. These grayscale inverted images are again saved according to a standardized naming convention at 2130, and then used as input to another image processing and visualization software program 2132 (Aperio ImageScope) for additional processing. A set of output files suitable for visualization are generated and saved at 2134.

### Example 16—Sample Staining Protocol

### Step 1: Dewax & Counterstain:

1. Deparaffinize slides: Xylene 2×5 min, 100% ETOH 2×2 min, 95% ETOH 2×2 min, 70%
ETOH 2×2 min, 50% ETOH 1×2 min, dH₂0 2×2 min;
2. Counterstain in 0.01% Methyl Green 3 mins, wash in H₂0 briefly;
3. Cover section (Cover Glass Signature Series, Thermo Scientific);
4. Scan the slides in Aperio;
5. Decover section gently;
6. Block endogenous peroxidase activity: 245 ml Methanol+5 ml 30%-H₂0₂ 30 min @ room temperature (RT);
7. Wash slides in dH₂0 (1×2 min) and TBST (2×2 min).

### Step 2: Antigen Retrieval:

1. Microwave treatment (Only for the 1st round, other Ag-retrieval solutions can be used in microwave, steamer, etc);
   1) Place slides in a plastic jar and fill it completely with Antigen Retrieval Citra #HK087-5K BioGenex 250 ml (or other Ag-retrieval solutions);
   2) Without covering by lid, place it in microwave and first bring the liquid to boiling point (45-80 sec at 100% power). Add 30 sec after bubbles clearly come up (Or check the temperature is over 90° C.). Then, microwave for an additional 15 mins at
   10% power;
   3) Allow slides to cool down at RT (15 min);
2. Wash slides in dH20 (1×2 min) and TBST (1×2 min).

### Step 3: Blocking:

1. Using PAP pen, “draw” around sections carefully, if needed;
2. Block section in Blocking buffer (5% normal goat serum, 2.5% BSA, 1×PBS) for 10 min @ RT (use 100-200 μl/section);
3. Aspirate blocking buffer.

### Step 4: Primary Antibody Incubation:

1. Apply Primary antibody (100-200 μl/section) to section. Incubate in humidified chamber at 30 min @RT or overnight @ 4 C. (This protocol should be optimized based on each antibody);
2. TBST wash, 3×2 min with agitation;

### Step 5: Introduction of Secondary-HRP:

1. Incubate slides in HistoFine(M/R/G) Simple Stain MAX PO (Nichirei Bioscience Inc) (1-2 drops/section) at RT for 30 mins;
2. TBST wash, 3×2 min with agitation.

### Step 6: Visualization & Scanning:

1. Pipette AEC solution (Vector Lab, SK-4200). Incubate 5-40 min (usually 20 min) @RT. Wash in TBST briefly;
2. Cover section (Cover Glass Signature Series, Thermo Scientific).

### Example 17

Sequential Multiplexed IHC Protocol. This Example describes adaptation of the methodology described in Example 16 to utilize an autostainer, in this Example, the Ventana BenchMark XT system.

### <Preparation>

1. The XT ultraView DAB v3 procedure is used, with the following selections:
   a. Paraffin (selected)
   b. Cell Conditioning (selected)
   c. Conditioner #2 (selected)*
   d. Mild CC2 (selected)
   e. Option (selected)
   f. Apply one drop of Option 1 and incubate for 32 min
   g. Ab Incubation Temperatures (selected)
   h. 37 C Ab Inc. (selected)
   i. Titration (selected)
   j. Hand Apply (Primary antibody) and incubate for 32 min
   *NOTE: for the first round of multiplexing, CC1 mild is used instead of CC2.
2. The Option 1 dispenser is filled with 10% NGS and 5% BSA in TBST.
3. After run has started, the dispensers UV DAB, UV DAB H2O2, and UV COPPER are removed from the carousel.
4. Antibodies are diluted to working strength with Dako antibody diluent, S0809, just prior to pipetting onto slides.
   <Staining> (Modified from Glass et al.)

### Step 1: Deparaffinization & Counterstain

1. Incubate slides at 58-60° C. for 30 min.
2. Deparaffinize slides: Xylene 2×5 min, 100% ETOH 2×2 min, 95% ETOH 2×2 min, 70% ETOH 2×2 min, 50% ETOH 1×2 min, diH₂0 2×2 min
3. Counterstain in Hematoxylin (Dako S3301) for 1 min, diH₂0 2 min
4. Cover section (Cover Glass Signature Series, Thermo Scientific) with TBST (0.1 M TRIS-HCl, pH 7.5, 0.15 M NaCl plus 0.05% Tween-20).
5. Scan the slides in Aperio.
6. Decover section gently following 1 min agitation in TBST.
   <Endogenous peroxidase blocking>
   a. Apply One Drop of UV INHIBITOR, Apply Coverslip, and Incubate for 4 Minutes (UV Inhibitor is 3% hydrogen peroxide to quench endogenous peroxidase activity)
   b. Rinse Slide With Reaction Buffer
   c. Adjust Slide Volume With Reaction Buffer

### Step 2: Antigen Retrieval/Heat Treatment

1. *****Select EZ Prep*****
2. *****Start Timed Steps*****
3. *****Mixers Off*****
4. Warmup Slide to 75 Deg C., and Incubate for 4 Minutes
5. Apply EZPrep Volume Adjust
6. Rinse Slide
7. Apply EZPrep Volume Adjust
8. Rinse Slide
9. Apply EZPrep Volume Adjust
10. Apply Coverslip
11. Warmup Slide to 76 Deg C., and Incubate for 4 Minutes
12. Rinse Slide
13. Apply Depar Volume Adjust
14. Apply Coverslip
15. Disable Slide Heater
16. *****Mixers On*****
17. [Short—8 Minute Conditioning] (i.e. antigen retrieval)
18. Rinse Slide
19. Apply Long Cell Conditioner #2
20. Apply CC Coverslip Long
21. *****Select SSC Wash*****
22. Warmup Slide to 95 Deg C., and Incubate for 8 Minutes
23. [Mild—36 Minute Conditioning] (i.e. antigen retrieval)
24. Apply Cell Conditioner #2
25. Apply CC Medium Coverslip No BB
26. Warmup Slide to 100 Deg C., and Incubate for 4 Minutes
27. Apply CC Medium Coverslip No BB
28. Apply Cell Conditioner #2
29. Apply CC Medium Coverslip No BB
30. Apply Cell Conditioner #2
31. Apply CC Medium Coverslip No BB
32. Apply Cell Conditioner #2
33. Apply CC Medium Coverslip No BB
34. Apply Cell Conditioner #2
35. Apply CC Medium Coverslip No BB
36. Apply Cell Conditioner #2
37. Apply CC Medium Coverslip No BB
38. Disable Slide Heater
39. Incubate for 8 Minutes
40. Rinse Slide With Reaction Buffer
41. Adjust Slide Volume With Reaction Buffer
42. Apply Coverslip
43. Rinse Slide With Reaction Buffer
44. Adjust Slide Volume With Reaction Buffer
45. *****Procedure Synchronization*****
46. Warmup Slide to 37 Deg C., and Incubate for 4 Minutes
47. Rinse Slide With Reaction Buffer
48. Adjust Slide Volume With Reaction Buffer

### Step 3: Protein Blocking

1. Apply One Drop of [OPTION 1] (Option), Apply Coverslip, and Incubate for [32 Minutes] (Option 1 contains 10% NGS and 5% BSA in TBST)
2. Rinse Slide With Reaction Buffer
3. Adjust Slide Volume With Reaction Buffer
4. Apply Coverslip
5. Warmup Slide to 37 Deg C., and Incubate for 4 Minutes
6. Rinse Slide With Reaction Buffer
7. Adjust Slide Volume With Reaction Buffer
8. Apply Coverslip

### Step 4: Primary Antibody Incubation

1. *****Hand Apply (Primary Antibody), and Incubate for [0 Hr 16 Min]***** (Instrument tray is opened up and primary antibody is hand pipetted onto each slide)
2. Rinse Slide With Reaction Buffer
3. Adjust Slide Volume With Reaction Buffer
4. Apply Coverslip
5. Warmup Slide to 37 Deg C., and Incubate for 4 Minutes
6. Rinse Slide With Reaction Buffer
7. Adjust Slide Volume With Reaction Buffer

### Step 5: Introduction of Secondary-HRP

1. Apply One Drop of UV HRP UNIV MULT, Apply Coverslip, and Incubate for 8 Minutes (ultraView Universal HRP Multimer contains a cocktail of HRP labeled antibodies (goat anti-mouse IgG, goat anti-mouse IgM, and goat anti-rabbit) (<50 μg/mL) in a buffer containing 10% casein with ProClin 300, a preservative)
2. Rinse Slide With Reaction Buffer
3. Adjust Slide Volume With Reaction Buffer
4. Apply Coverslip
5. Rinse Slide With Reaction Buffer
6. Adjust Slide Volume With Reaction Buffer
7. Apply One Drop of UV DAB and One Drop of UV DAB H202, Apply Coverslip, Incubate for 8 Minutes (NOTE: these dispensers were removed from carousel so nothing is dispensed)
8. Rinse Slide With Reaction Buffer
9. Adjust Slide Volume With Reaction Buffer
10. Apply One Drop of UV COPPER, Apply Coverslip, and Incubate for 4 Minutes (NOTE: these dispensers were removed from carousel so nothing is dispensed.)
11. Rinse Slide With Reaction Buffer
12. Apply Coverslip
13. Disable Slide Heater
14. *****Select Optional Wash*****
15. *****Select SSC Wash*****
16. *****Start Timed Steps*****
17. Rinse Slide With Reaction Buffer
<After the run on the Ventana, slides are removed and washed briefly in Dawn dish soap and distilled water, then rinsed in distilled water.>

### Step 6: Visualization & Scanning

1. Pipette AEC solution (Vector Lab, SK-4200). Incubate 5-40 min (usually 20 min) @RT. Wash in diH20 and TBST briefly.
2. Cover section (Cover Glass Signature Series, Thermo Scientific) with TBST
3. Scan the slides in Aperio.
4. Decover section gently following 1 min agitation in TBST

### Step 7: Removal of AEC/Antibodies

1. AEC wash: diH20 briefly, 70% ETOH briefly, 100% ETOH with agitation until signal-clearance (usually 2-3 min+add 30 sec after AEC becomes invisible), 70% ETOH 1×1 min, 30% ETOH 1×1 min, dH20 wash 4× briefly (make sure for perfect elimination of EtOH), TBST 1×1 mins. Slides are either loaded onto the Ventana for another round of IHC (Step 5) or stored in TBST at 4 C.>
2. Heat treatment according to Step 2.
3. Wash slides in dH20 (1×1 min) and TBST (1×1 min).
For multiplex, repeat Steps 3 to 7.

### <Image Processing & Analysis>

### Step 1: Composition of Sequentially Scanned Images

1. Select a target cell or structure which can be utilized for alignment. Extract jpg images (usually 200 square pixels) from each scanned image.
2. Run CellProfiler with “Alignment_Batch” pipeline (Produced by Vahid Azimi & Rohan Borkar, Intel Corporation).
3. Identify the target cell/structure in each image, and draw the outline of cells following press “F”. Repeat this cycle to all images.
4. Input alignment information from CMV files to an excel file named “alignment”. Extract images as TIFF or JPG files (max: 20,000 pixel square).

### Step 2: Visualization

1. Open extracted images in ImageJ. Run the macro “Color Deconvolution” and select H/AEC (Ref [2]).
2. Change images for gray scaled and inverted. Adjust Brightness/Contrast if needed.
3. Open images in the Aperio Image Scope, and merge all channeled images by “Fuse Images”. Mis-alignment among images can be also adjusted in the Aperio Image Scope.

### Step 3: Quantification

1. Go back to Visualization Step 2-2. Convert inverted gray-scaled images to brown-colored (Merge channels[cyan and gray] & Invert).
2. Merge brown-colored image and counter stain image by using ImageCalculator (Min).
3. Open images in Aperio Image Scope. Run the algorithm “membranous count v9”.
4. Export data to excel spreadsheet. Draw heatmaps and bar graphs.
5. For counting double/triple positive cells, two or three different single-channel images can be merged by using ImageCalculator in ImageJ.

### Example 17 References: Glass et al., J Histochem Cytochem 57: 899-905; Ruifrok & Johnston, Anal Quant Cytol Histol 23: 291-299, 2001.

### Example 18—Protocol for Multiplexed IHC 2.0

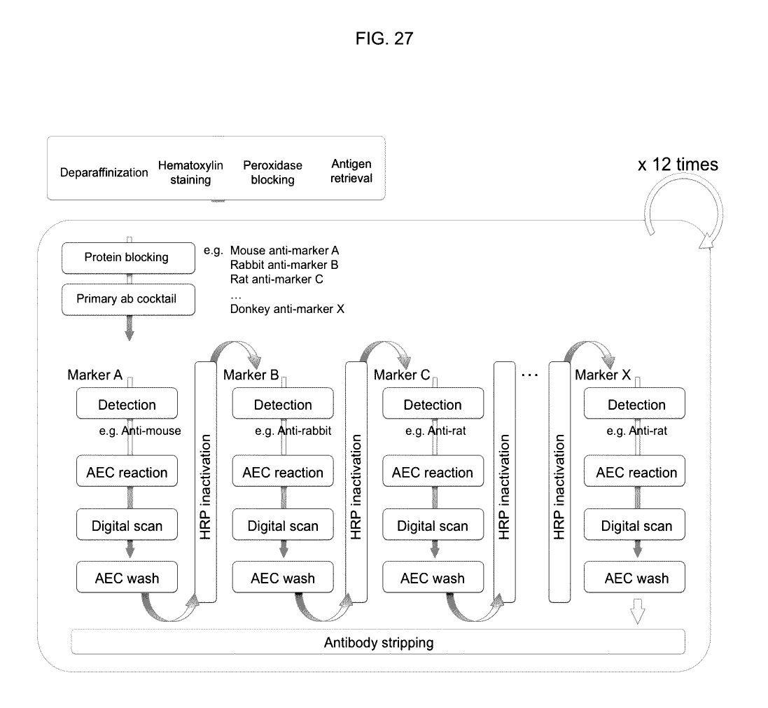

### <Staining> (See FIG. 27)

### Step 1: Deparaffinization & Counterstain

1. Incubate slides at 58-60° C. for 30 min.
2. Deparaffinize slides: Xylene 2×5 min, 100% ETOH 2×2 min, 95% ETOH 2×2 min, 70% ETOH 2×2 min, 50% ETOH 1×2 min, diH20 2×2 min
3. Counterstain in Hematoxylin (Dako S3301) for 1 min, diH20 2 min
4. Cover section (Cover Glass Signature Series, Thermo Scientific) with TBST (0.1 M TRIS-HCl, pH 7.5, 0.15 M NaCl plus 0.05% Tween-20).
5. Scan the slides in Aperio.
6. Decover section gently following 1 min agitation in TBST.
7. Block endogenous peroxidase activity: incubate slides in 0.6% H202 in Methanol for 30 min @ RT.
8. Wash slides in dH20 (1×1 min) and TBST (1×1 min).

### Step 2: Heat Treatment

9. Microwave treatment
   1) Place slides in a plastic jar and fill it with Antigen Retrieval Citra #HK087-5K BioGenex 250 ml.
   2) Without covering by lid, place it in microwave and first bring the liquid to boiling point (60-90 sec at 100% power). Add 10-15 sec at 100%×2 so that bubbles clearly come up to make sure over 90° C. Then, microwave for an additional 15 mins at reduced (e.g. 10-20%) power. This process should be optimized depending on microwave specs to maintain over 90° C. for 15 min.
   3) Allow slides to cool down at room temperature (15 min).
10. Wash slides in diH₂0 (1×1 min) and TBST (1×1 min).

### Step 3: Blocking

11. Using PAP pen only if inevitable. No usage of PAP pen is highly recommended for good coverslipping procedure.
12. Block section in Blocking buffer (5% normal goat serum, 2.5% BSA, 1×PBS) for 10 min @ RT (use about 100-200 μl/section).
13. Aspirate blocking buffer.

### Step 4: Primary Antibody Incubation

14. Apply Primary antibody (100-200 μl/section) to section (dilute in 0.5×block buffer). Incubate in humidified chamber at 30 min @RT or overnight @ 4° C. (This protocol should be optimized based on each antibody). Instead of applying single antibody, antibody cocktail derived from different host species can be applied in this step.
15. TBST wash, 3×2 min with agitation.

### Step 5: Introduction of Secondary-HRP

16. Incubate slides in primary antibody-targeting secondary antibodies with HRP-polymer (e.g. HistoFine(M/R/G) Simple Stain MAX PO, Nichirei Bioscience Inc, 1-2 drops/section) at RT for 30 mins. If needed, this step can be replaced with biotinylated secondary antibodies followed by Avidin-Biotin Complex method.
17. TBST wash, 3×2 min with agitation. Wash slides in diH₂0 briefly. Do not mix TBST and AEC solution.

### Step 6: Visualization & Scanning (Glass et al., (2009) J Histochem Cytochem 57: 899-905)

18. Pipette AEC solution (Vector Lab, SK-4200). Incubate 5-40 min (usually 20 min) @RT. Wash in diH₂0 and TBST briefly.
19. Cover section (Cover Glass Signature Series, Thermo Scientific) with TBST
20. Scan the slides in Aperio.
21. Decover section gently following 1 min agitation in TBST

### Step 7: Removal of AEC

22. AEC wash: diH₂0 briefly, 70% ETOH briefly, 100% ETOH with agitation until signal-clearance (usually 2-3 min+add 30 sec after AEC becomes invisible), 70% ETOH 1×1 min, 30% ETOH 1×1 min, dH₂0 wash 4× briefly (make sure for perfect elimination of EtOH), TBST 1×1 min
   <For, HRP inactivation, go to Step 8.>
   <For antibody stripping, go back to Step 2 as a new cycle>

### Step 8: HRP Inactivation

23. Incubate slides in 0.6% H₂0₂ in Methanol for 30 min @ RT.
24. Wash slides in dH₂0 (1×1 min) and TBST (1×1 min).
25. Go back to Step 3 as a new cycle. If antibody cocktail was applied in Step 4, go back to Step 5 and select appropriate secondary antibody for target primary antibody.

### <Image Processing & Analysis>

### Step 1: Composition of Sequentially Scanned Images

1. Select a target cell or structure which can be utilized for alignment. Extract jpg images (usually 200 square pixels) from each scanned image.
2. Run CellProfiler with “Alighment_Batch” pipeline (Produced by Vahid Azimi & Rohan Borkar, Intel Corporation).
3. Identify the target cell/structure in each image, and draw the outline of cells following press “F”. Repeat this cycle to all images.
4. Input alignment information from CMV files to an excel file named “alignment”. Extract images as TIFF or JPG files (max: 20,000 pixel square).

### Step 2: Visualization

1. Open extracted images in ImageJ. Run the macro “Color Deconvolution” and select H/AEC (Ruifrok & Johnston, Anal Quant Cytol Histol 23: 291-299, 2001).
2. Change images for gray scaled and inverted. Adjust Brightness/Contrast if needed.
3. Open images in the Aperio Image Scope, and merge all channeled images by “Fuse Images”. Mis-alignment among images can be also adjusted in the Aperio Image Scope.

### Step 3: Quantification

1. Go back to Step2-2. Convert inverted gray-scaled images to brown-colored (Merge channels[cyan and gray] & Invert).
2. Merge brown-colored image and counter stain image by using ImageCalculator (Min).
3. Open images in Aperio Image Scope. Run the algorithm “membranous count v9”.
4. Export data to excel spreadsheet. Draw heatmaps and bar graphs.
5. For counting double/triple positive cells, two or three different single-channel images can be merged by using ImageCalculator in ImageJ.

1. A method of performing immunohistochemistry on a formalin fixed paraffin embedded tissue section, the method comprising:
contacting the section with a first tissue antigen specific antibody that specifically binds a tissue antigen wherein the first tissue antigen specific antibody optionally comprises a first enzyme label;
contacting the section with an enzyme-labeled antibody that specifically binds the first tissue antigen specific antibody if the first tissue antigen specific antibody does not comprise a first enzyme label;
contacting the section with a first colorimetric substrate of the first enzyme label, thereby visualizing one or more cells that express the first tissue antigen;
generating a first digital image of all or part of the section; thereby completing a first staining cycle;
heating the section to at least 90° C. for a sufficient time to remove the first tissue antigen specific antibody and the first labeled antibody from the one or more cells that express the first tissue specific antigen;
contacting the section with a second tissue antigen specific antibody that specifically binds a tissue antigen wherein the second tissue antigen specific antibody optionally comprises a second enzyme label;
contacting the section with an enzyme-labeled antibody that specifically binds the second tissue antigen specific antibody if the second tissue antigen specific antibody does not comprise a second enzyme label;
contacting the section with a second colorimetric substrate of the second enzyme label, and generating a second digital image of the section, thereby completing a second staining cycle
provided that the heating is performed between the first staining cycle and the second staining cycle.

2. The method of claim 1 further comprising:
heating the section to at least 90° C. after the second staining cycle;
contacting the section with a third tissue antigen specific antibody that specifically binds a tissue antigen wherein the third tissue antigen specific antibody optionally comprises a third enzyme label;
contacting the section with an enzyme-labeled antibody that specifically binds the third tissue antigen specific antibody if the third tissue antigen specific antibody does not comprise a third enzyme label;
contacting the section with a third colorimetric substrate of the third enzyme label, and generating a third digital image of the section, thereby completing a third staining cycle;
heating the section to at least 90° C. after the third staining cycle;
contacting the section with a fourth tissue antigen specific antibody that specifically binds a tissue antigen wherein the fourth tissue antigen specific antibody optionally comprises a fourth enzyme label;
contacting the section with an enzyme-labeled antibody that specifically binds the fourth tissue antigen specific antibody if the fourth tissue antigen specific antibody does not comprise a fourth enzyme label;
contacting the section with a fourth colorimetric substrate of the fourth enzyme label, and generating a fourth digital image of the section, thereby completing a fourth staining cycle;
heating the section to at least 90° C. after the fourth staining cycle;
contacting the section with a fifth tissue antigen specific antibody that specifically binds a tissue antigen wherein the fifth tissue antigen specific antibody optionally comprises a fifth enzyme label;
contacting the section with an enzyme-labeled antibody that specifically binds the fifth tissue antigen specific antibody if the fifth tissue antigen specific antibody does not comprise a fifth enzyme label,
contacting the section with a fifth colorimetric substrate of the fifth enzyme label, and generating a fifth digital image of the section, thereby completing a fifth staining cycle;
heating the section to at least 90° C. after the fifth staining cycle;
contacting the section with a sixth tissue antigen specific antibody that specifically binds a tissue antigen wherein the sixth tissue antigen specific antibody optionally comprises a sixth enzyme label;
contacting the section with an enzyme-labeled antibody that specifically binds the sixth tissue antigen specific antibody if the sixth tissue antigen specific antibody does not comprise a sixth enzyme label;
contacting the section with a sixth colorimetric substrate of the sixth enzyme label, and generating a sixth digital image of the section, thereby completing a sixth staining cycle;
heating the section to at least 90° C. after the sixth staining cycle;
contacting the section with a seventh tissue antigen specific antibody that specifically binds a tissue antigen wherein the seventh tissue antigen specific antibody optionally comprises a seventh enzyme label;
contacting the section with an enzyme-labeled antibody that specifically binds the seventh tissue antigen specific antibody if the seventh tissue antigen specific antibody does not comprise a seventh enzyme label;
contacting the section with a seventh colorimetric substrate of the seventh enzyme label, and generating a seventh digital image of the section, thereby completing a seventh staining cycle.

3. The method of claim 2 further comprising staining the section with hematoxylin, eosin, periodic acid-Schiff's stain, Mason's Trichrome, Gomori Trichrome, silver salts, Wright's, or Giemsa, thereby generating a structure-stained digital image.

4. The method of claim 3 further comprising coregistering the first, second, third, fourth, fifth, sixth and seventh digital images and merging the first, second, third, fourth, fifth, sixth, and seventh digital images into a composite image.

5. The method of claim 4 wherein coregistering the first, second, third, fourth, fifth, sixth, and seventh digital images comprises calculating a transformation that brings each of said digital images into alignment with the structure-stained digital image.

6. The method of claim 5, wherein calculating a transformation that brings each of the first, second, third, fourth, fifth, sixth, and seventh digital images digital images into alignment with the structure-stained digital image comprises calculating a delta-X and delta-Y offset among the first, second, third, fourth, fifth, sixth, and seventh digital images relative to the structure-stained digital image.

7. The method of claim 6 where coregistering the first, second, third, fourth, fifth, sixth, and seventh digital images comprises calculating a delta-X and delta-Y location among the first, second, third, fourth, fifth, sixth, and seventh digital images.

8. The method of claim 4 further comprising performing cell segmentation on the structure-stained digital image and on the first, second, third, fourth, fifth, sixth, and seventh digital images.

9. The method of claim 8 where performing cell segmentation comprises:
converting the structure-stained digital image into a grayscale structure-stained image;
inverting the pixel intensity of the grayscale structure-stained image, thereby creating an inverted grayscale structure-stained image;
differentiating the foreground and background pixels of the inverted grayscale structure-stained image using a thresholding method;
applying a watershed segmentation algorithm to the inverted grayscale structure-stained image, thereby generating a set of cell object masks;
extracting an AEC color channel from the first digital image;
overlaying the set of cell object masks on the AEC color channel, thereby creating a first masked protein stained cellular image;
measuring the intensity of one or more pixels of the first masked protein stained cellular image; and
generating a color map by assigning a number to each pixel within each cell of the first masked protein stained cellular image.

10. The method of claim 9 further comprising creating second, third, fourth, fifth, sixth, and seventh masked protein stained cellular images from the second, third, fourth, fifth, sixth and seventh digital images respectively by
extracting an AEC color channel from the second, third, fourth, fifth, sixth and seventh digital images respectively;
overlaying the set of cell object masks on the AEC color channels extracted from the AEC color channel from the second, third, fourth, fifth, sixth and seventh digital images respectively thereby creating second, third, fourth, fifth, sixth and seventh masked protein stained cellular images respectively;
measuring the intensity of one or more pixels of the second, third, fourth, fifth, sixth and seventh masked protein stained cellular images respectively; and
generating a color map by assigning a number to each pixel within each cell of the second, third, fourth, fifth, sixth and seventh masked protein stained cellular images.

11. The method of claim 4 further comprising performing tissue segmentation on the section.

12. The method of claim 11, wherein performing tissue segmentation on the section comprises defining a region of interest in the first digital image, where the region of interest comprises at least a portion of the section that includes tissue but excludes non-tissue regions; applying an image-cleaning algorithm to the region of interest, thereby generating a cleaned region of interest; identifying a tumor nest region within the cleaned region of interest; applying an image cleaning algorithm to the tumor nest region, thereby generating a cleaned tumor nest region; subtracting the cleaned tumor nest region from the cleaned region of interest, thereby generating a stromal region; cropping the structure-stained image to fit to the cleaned tumor nest region and the stromal region; and analyzing the cleaned tumor nest region and the stromal region by image cytometry.

13. The method of claim 1 further comprising maintaining the section at at least 90° C. for at least 15 minutes.

14. The method of claim 1 where heating the section comprises heating the section in a microwave oven or placing the section in a heat bath.

15. The method of claim 1 where heating the section is performed in a citrate buffer of pH 5.5-6.5.

16. The method of claim 1 where the first and/or second enzyme label is selected from horseradish peroxidase, alkaline phosphatase, glucose oxidase, and β-galactosidase.

17. The method of claim 16 where first and/or second enzyme label comprises horseradish peroxidase and the associated first and/or second colorimetric substrate comprises ABTS (2,2′-azino-bis(3-ethylbenzothiazoline-6-sulphonic acid)), OPD (o-phenylenediamine dihydrochloride), TMB (tetramethylbenzidine), 4CN (4-chloro-1-napthol), DAB (3,3′-diaminobenzidine), or AEC (3-amino-9-ethylcarbazole); where the first and/or second enzyme label comprises alkaline phosphatase and the associated first and/or second colorimetric substrate comprises BCIP (5-bromo-4-chloro-3-indolyl-phosphate), and/or NBT (nitro-blue tetrazolium chloride); where the first and/or second enzyme label comprises glucose oxidase and where the associated first and/or second colorimetric substrate comprises NBT; and where the first and/or second enzyme label comprises β-galactosidase and the associated first and/or second colorimetric substrate comprises X-Gal (5-bromo-4-chloro-3-indolyl-β-D-galactopyranoside).

18. The method of claim 2 further comprising:
heating the section to at least 90° C. after the seventh staining cycle;
contacting the section with an eighth tissue antigen specific antibody that specifically binds a tissue antigen wherein the eighth tissue antigen specific antibody optionally comprises an eighth enzyme label;
contacting the section with an enzyme-labeled antibody that specifically binds the eighth tissue antigen specific antibody if the eighth tissue antigen specific antibody does not comprise an eighth enzyme label;
contacting the section with an eighth colorimetric substrate of the eighth enzyme label, and generating an eighth digital image of the section, thereby completing an eighth staining cycle;
heating the section to at least 90° C. after the eighth staining cycle;
contacting the section with a ninth tissue antigen specific antibody that specifically binds a tissue antigen;
contacting the section with a ninth tissue antigen specific antibody that specifically binds a tissue antigen wherein the ninth tissue antigen specific antibody optionally comprises a ninth enzyme label;
contacting the section with an enzyme-labeled antibody that specifically binds the ninth tissue antigen specific antibody if the ninth tissue antigen specific antibody does not comprise a ninth enzyme label;
contacting the section with a ninth colorimetric substrate of the ninth enzyme label, and generating a ninth digital image of the section, thereby completing a ninth staining cycle;
heating the section to at least 90° C. after the ninth staining cycle;
contacting the section with a tenth tissue antigen specific antibody that specifically binds a tissue antigen wherein the tenth tissue antigen specific antibody optionally comprises a tenth enzyme label;
contacting the section with an enzyme-labeled antibody that specifically binds the tenth tissue antigen specific antibody if the tenth tissue antigen specific antibody does not comprise a tenth enzyme label;
contacting the section with a tenth colorimetric substrate of the tenth enzyme label, and generating a tenth digital image of the section, thereby completing a tenth staining cycle;
heating the section to at least 90° C. after the tenth staining cycle;
contacting the section with an eleventh tissue antigen specific antibody that specifically binds a tissue antigen wherein the eleventh tissue antigen specific antibody optionally comprises an eleventh enzyme label;
contacting the section with an enzyme-labeled antibody that specifically binds the eleventh tissue antigen specific antibody if the eleventh tissue antigen specific antibody does not comprise an eleventh enzyme label;
contacting the section with an eleventh colorimetric substrate of the eleventh enzyme label, and generating an eleventh digital image of the section, thereby completing an eleventh staining cycle;
heating the section to at least 90° C. after the eleventh staining cycle;
contacting the section with a twelfth tissue antigen specific antibody that specifically binds a tissue antigen wherein the twelfth tissue antigen specific antibody optionally comprises a twelfth enzyme label;
contacting the section with an enzyme-labeled antibody that specifically binds the twelfth tissue antigen specific antibody if the twelfth tissue antigen specific antibody does not comprise a twelfth enzyme label; and
contacting the section with a twelfth colorimetric substrate of the twelfth enzyme label, and generating a twelfth digital image of the section, thereby completing a twelfth staining cycle.

19. The method of claim 18 further comprising coregistering the eighth, ninth, tenth, eleventh, and twelfth digital images and merging the eighth, ninth, tenth, eleventh, and twelfth digital images into the composite image.

20. The method of claim 18 further comprising coregistering the eighth, ninth, tenth, eleventh, and twelfth digital images and merging the eighth, ninth, tenth, eleventh, and twelfth digital images into the composite image.

21. The method of claim 18 comprising completing 36-60 staining cycles.

22. The method of claim 18 where the first tissue specific antibody specifically binds PD-1; the second tissue specific antibody specifically binds CD3; the third tissue specific antibody specifically binds RORγT, the fourth tissue specific antibody specifically binds CD56, the fifth tissue specific antibody specifically binds CD8, the sixth tissue specific antibody specifically binds Tbet, the seventh tissue specific antibody specifically binds GATA3, the eighth tissue specific antibody specifically binds Foxp3, the ninth tissue specific antibody specifically binds PD-L1, the tenth tissue specific antibody specifically binds CD20, the eleventh tissue specific antibody specifically binds CD45, and the twelfth tissue specific antibody specifically binds EpCAM or p16.

23. The method of claim 18 where the first, second, third, fourth, fifth, sixth, seventh, eighth, ninth, tenth, eleventh, and twelfth enzyme labels comprise horseradish peroxidase, where the first, second, third, fourth, fifth, sixth, seventh, eighth, ninth, tenth, eleventh, and twelfth colorimetric substrates comprise 3-amino-9-ethylcarbazole (AEC) and where the colorimetric substrate is allowed to incubate for 20 minutes in the first staining cycle, for 20 minutes in the second staining cycle, for 10 minutes in the third staining cycle, for 40 minutes in the fourth staining cycle, for 20 minutes in the fifth staining cycle, for 20 minutes in the sixth staining cycle, for 40 minutes in the seventh staining cycle, for 20 minutes in the eighth staining cycle, for 40 minutes in the ninth staining cycle, for 20 minutes in the tenth staining cycle, for 40 minutes in the eleventh staining cycle and for 10 minutes in the twelfth staining cycle if the twelfth tissue specific antibody specifically binds EpCAM or for 20 minutes in the twelfth staining cycle if the twelfth tissue specific antibody specifically binds p16.

24. The method of claim 18 wherein the first tissue specific antibody specifically binds tryptase, the second tissue specific antibody specifically binds CD68, the third tissue specific antibody specifically binds CSF1R, the fourth tissue specific antibody specifically binds DC-SIGN, the fifth tissue specific antibody specifically binds CD66b, the sixth tissue specific antibody specifically binds CD83, the seventh tissue specific antibody specifically binds CD163, the eighth tissue specific antibody specifically binds Class II MHC, the ninth tissue specific antibody specifically binds PD-L1, the tenth tissue specific antibody specifically binds CD3, CD20, and CD56, the eleventh tissue specific antibody specifically binds CD46, and the twelfth tissue specific antibody specifically binds EpCAM or p16.

25. The method of claim 24 where the first, second, third, fourth, fifth, sixth, seventh, eighth, ninth, tenth, eleventh, and twelfth enzyme labels comprise horseradish peroxidase, where the first, second, third, fourth, fifth, sixth, seventh, eighth, ninth, tenth, eleventh, and twelfth colorimetric substrates comprise 3-amino-9-ethylcarbazole (AEC) and wherein the colorimetric substrate is allowed to incubate for 20 minutes in the first staining cycle, for 20 minutes in the second staining cycle, for 10 minutes in the third staining cycle, for 20 minutes in the fourth staining cycle, for 20 minutes in the fifth staining cycle, for 20 minutes in the sixth staining cycle, for 20 minutes in the seventh staining cycle, for 10 minutes in the eighth staining cycle, for 40 minutes in the ninth staining cycle, for 20 minutes in the tenth staining cycle, for 40 minutes in the eleventh staining cycle and for 10 minutes in the twelfth staining cycle if the twelfth tissue specific antibody specifically binds EpCAM or for 20 minutes in the twelfth staining cycle if the twelfth tissue specific antibody specifically binds p16.

26. The method of claim 1 where the first tissue antigen specific antibody and the first labeled antibody are the same antibody.

27. The method of claim 1 where the tissue section is provided on a tissue microarray.

28. The method of claim 1 further comprising destaining the colorimetric substrate.

29. The method of claim 28 where destaining the colorimetric substrate is performed using an ethanol gradient.

30. The method of claim 1 where one or more of:
contacting the section with the first tissue antigen specific antibody
contacting the section with the first labeled antibody;
contacting the section with the first colorimetric substrate;
destaining the colorimetric substrate;
or heating the section to at least 90° C.
is performed through an automated system.

31. The method of claim 30 where the automated system performs methodology comprising automated liquid handling, movement of one or more slides to which the section is affixed via a robotic arm, or automated filling of a chamber.

32. The method of claim 1 comprising automated heating of the section to at least 95° C.
